(19) United States
(12) Patent Application Publication
(10) Pub. No.: US 2024/0368190 A1
(43) Pub. Date: Nov. 7, 2024

(54) SYNTHESIS OF RAS INHIBITORS

(71) Applicant: Revolution Medicines, Inc., Redwood City, CA (US)

(72) Inventors: Shaoling LI, Sunnyvale, CA (US); Suresh Manthati, Sunnyvale, CA (US); Ross Wang, Fremont, CA (US); Steve Ballmer, Burlingame, CA (US); Xiaojun Huang, San Jose, CA (US)

(21) Appl. No.: 18/634,098

(22) Filed: Apr. 12, 2024

Related U.S. Application Data
(60) Provisional application No. 63/459,435, filed on Apr. 14, 2023.

(51) Int. Cl. C07D 513/22; C07C 51/09; C07C 51/21; C07C 67/30; C07C 227/16; C07C 227/18; C07C 229/22; C07C 253/30; C07D 277/30; C07D 309/32; C07D 401/04; C07D 417/06; C07F 5/04

(52) U.S. Cl. CPC C07D 513/22; C07C 51/09; C07C 51/27; C07C 51/295; C07C 67/30; C07C 227/16; C07C 253/00; C07C 271/00; C07C 303/28; C07D 277/32; C07D 309/32; C07D 401/04; C07D 417/06; C07D 417/14; C07F 3/06; C07F 5/025; C07F 5/04; C07C 2601/02

(57) ABSTRACT

The present invention relates to Ras inhibitors and to methods for preparing Ras inhibitors.

### BACKGROUND

The vast majority of small molecule drugs act by binding a functionally important pocket on a target protein, thereby modulating the activity of that protein. For example, cholesterol-lowering drugs known as statins bind the enzyme active site of HMG-CoA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. Bojadzic and Buchwald, Curr Top Med Chem 18: 674-699 (2019). The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras, and N-Ras) play an essential role in various human cancers and are therefore appropriate targets for anticancer therapy. Indeed, mutations in Ras proteins account for approximately 30% of all human cancers in the United States, many of which are fatal Dysregulation of Ras proteins by activating mutations, overexpression or upstream activation is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state (Ras(ON)), leading to oncogenic MAPK signaling. Notably, Ras exhibits a picomolar affinity for GTP, enabling Ras to be activated even in the presence of low concentrations of this nucleotide. Mutations at codons 13 (e.g., G13C) and 61 (e.g., Q61K) of Ras are also responsible for oncogenic activity in some cancers.

Despite extensive drug discovery efforts against Ras during the last several decades, only two agents targeting the K-Ras G12C mutant have been approved in the U.S. (sotorasib and adagrasib). Additional efforts are needed to uncover additional medicines for cancers driven by the various Ras mutations, and there remains a need for convenient, scalable synthetic methods thereto.

### SUMMARY OF THE INVENTION

The invention features methods of preparing Compound A, intermediates useful in the synthesis of Compound A, and methods of preparing the intermediates. Compound A, a RAS inhibitor, has the following structure:

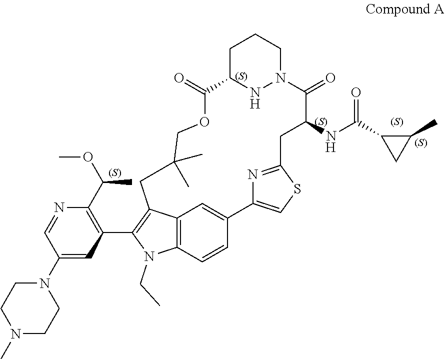

In a first aspect, the disclosure provides a method of preparing Compound 1:

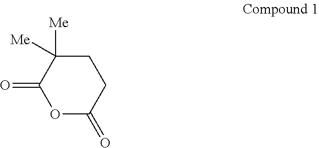

the method including:
a) reacting Compound 1a and Compound 1 b to form Compound 1c:

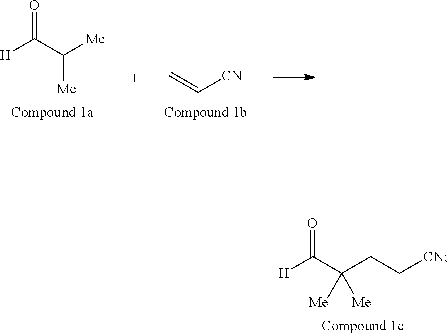

b) oxidizing and hydrolyzing Compound 1c to form Compound 1d:

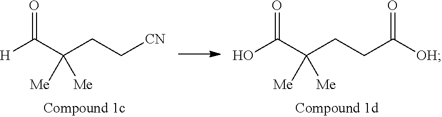 [SMILES: CC(C)(C=O)CCC#N ; CC(C)(CCC(=O)O)C(=O)O]

and
c) cyclizing Compound 1d to form Compound 1:

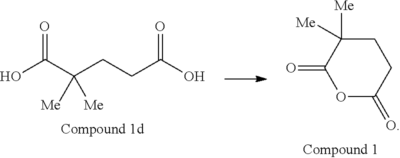 [SMILES: CC(C)(CCC(=O)O)C(=O)O ; CC1(C)CCC(=O)OC1=O]

In some embodiments, the reacting step (a) includes contacting Compound 1a and Compound 1b with a base. In some embodiments, the base is sodium hydroxide. In some embodiments, the reacting step (a) is carried out in the presence of hydroquinone.

In some embodiments, the oxidizing and hydrolyzing step (b) is carried out in the presence of sulfuric acid and nitric acid. In some embodiments, the oxidizing and hydrolyzing step (b) includes a first step of oxidizing Compound 1c to Compound 1e and a second step of hydrolyzing Compound 1e to Compound 1d:

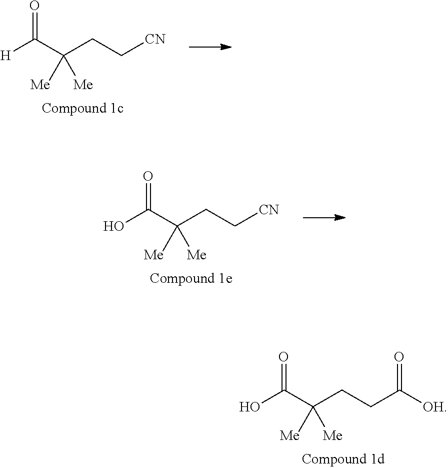 [SMILES: CC(C)(C=O)CCC#N ; CC(C)(CCC#N)C(=O)O ; CC(C)(CCC(=O)O)C(=O)O]

In some embodiments, the oxidizing step includes contacting NaClO₂ and Compound 1c. In some embodiments, the hydrolyzing step includes contacting potassium hydroxide and Compound 1e. In some embodiments, the second step further includes protonating Compound 1d by contacting the reaction with hydrochloric acid.

In some embodiments of the method of preparing Compound 1, the cyclizing step (c) includes contacting acetic anhydride and Compound 1d.

In some embodiments, the method further includes purifying Compound 1 by decolorization with activated carbon. In some embodiments, Compound 1 is purified by recrystallization. In some embodiments, the recrystallization is repeated more than once. In some embodiments, the recrystallization is carried out in methyl tert-butyl ether and n-heptane.

In another aspect, the disclosure provides a compound of Formula II:

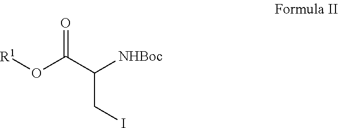

or a salt thereof, wherein R¹ is optionally substituted C₁-C₆ alkyl, optionally substituted 3- to 10-membered cycloalkyl, or optionally substituted C₆-C₁₀ aryl. In some embodiments, R¹ is optionally substituted C₁-C₆ alkyl (e.g., methyl).

In some embodiments, the compound has the structure of Formula IIa:

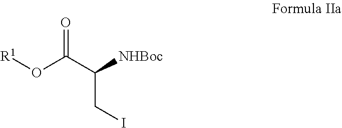

or a salt thereof, wherein R¹ is optionally substituted C₁-C₆ alkyl, optionally substituted 3- to 10-membered cycloalkyl, or optionally substituted C₆-C₁₀ aryl. In some embodiments, R¹ is optionally substituted C₁-C₆ alkyl (e.g., methyl).

In a further aspect, the disclosure provides a method of preparing Compound 2a. The method includes:
a) esterifying Compound 2b to form Compound 2c:

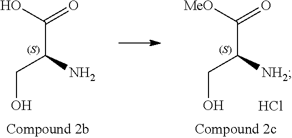

b) protecting and tosylating Compound 2c to form Compound 2d:

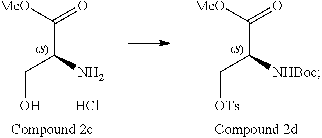

and
c) iodinating Compound 2d to form Compound 2a:

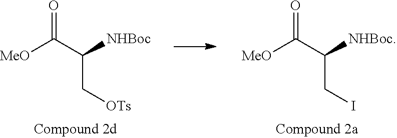 [SMILES: COC(=O)[C@H](COS(=O)(=O)c1ccc(C)cc1)NC(=O)OC(C)(C)C ; COC(=O)[C@H](CI)NC(=O)OC(C)(C)C]

In some embodiments of the method of preparing Compound 2a, the esterifying step (a) includes contacting an protic solvent (e.g., a methanol solution) of thionyl chloride and Compound 2b.

In some embodiments, the protecting and tosylating step (b) includes a first step of protecting Compound 2c to form Compound 2e and a second step of tosylating Compound 2e to form Compound 2d:

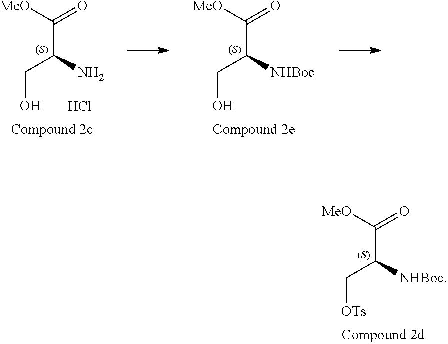

In some embodiments, the first protecting step includes contacting di-tert-butyl dicarbonate and Compound 2c, and the second tosylating step includes contacting tosyl chloride and Compound 2e.

In some embodiments, the iodinating step (c) includes contacting compound 2d with sodium iodide.

In an aspect, the disclosure provides a method of preparing Compound 3:

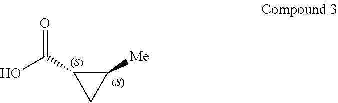

The method includes:
a) contacting Compound 3a and Compound 3b in the presence of a base to form Compound 3c:

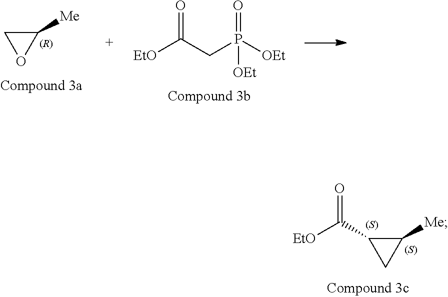

and
b) hydrolyzing Compound 3c to form Compound 3:

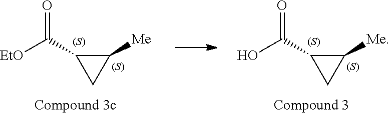

In some embodiments, the base of step (a) is n-butyllithium. In some embodiments, the contacting step (a) is carried out using a flow process.

In another aspect, the disclosure provides a compound having the structure of Compound 4:

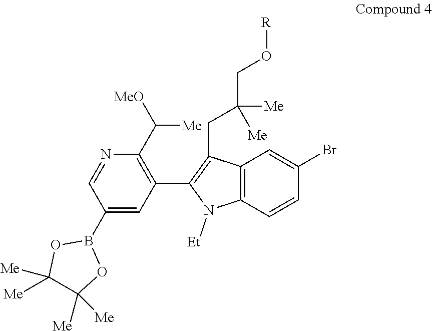

or a salt thereof, wherein R is H or

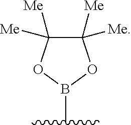 [SMILES: *B1OC(C)(C)C(C)(C)O1]

In some embodiments, R is H. In some embodiments, R is

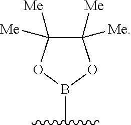 [SMILES: *B1OC(C)(C)C(C)(C)O1]

In some embodiments, the compound has the structure of Formula IIIa:

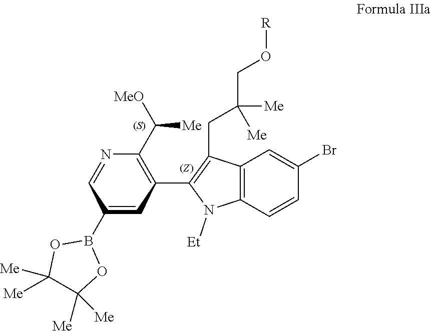

or a salt thereof, wherein R is H or

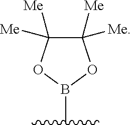 [SMILES: *B1OC(C)(C)C(C)(C)O1]

In some embodiments, R is H. In some embodiments, R is

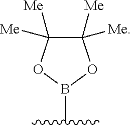 [SMILES: *B1OC(C)(C)C(C)(C)O1]

In yet another aspect, the disclosure provides a compound having the structure of Compound 5:

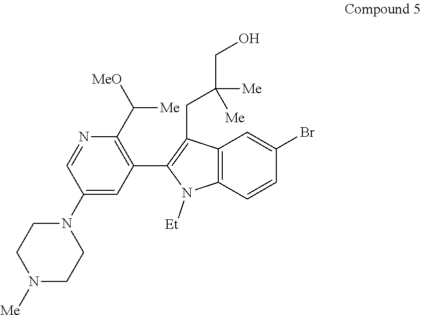 [SMILES: CCn1c(-c2cc(N3CCN(C)CC3)cnc2C(C)OC)c(CC(C)(C)CO)c2cc(Br)ccc21]

or a salt thereof.

In some embodiments, the compound has the structure of Compound 5a:

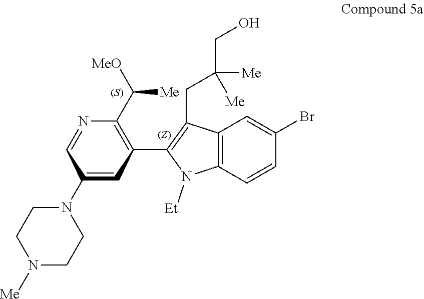

or a salt thereof.

In still another aspect, the disclosure provides a compound having the structure of Compound 6:

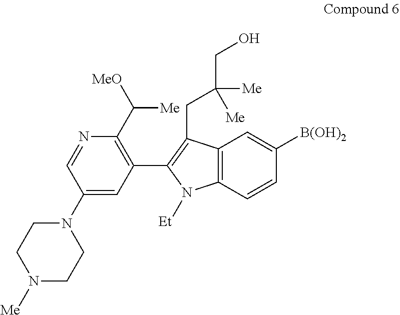 [SMILES: CCn1c(-c2cc(N3CCN(C)CC3)cnc2C(C)OC)c(CC(C)(C)CO)c2cc(B(O)O)ccc21]

or a salt thereof.

In some embodiments, the compound has the structure of Compound 6a:

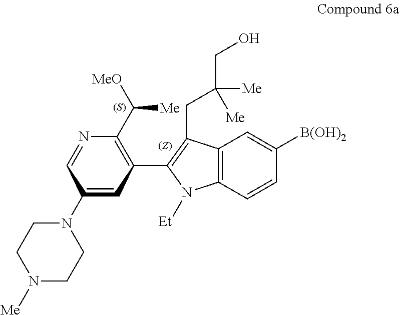

or a salt thereof.

In another aspect, the disclosure provides a method of preparing Compound 6a. The method includes:
a) borylating Compound 7 to form Compound 4a:

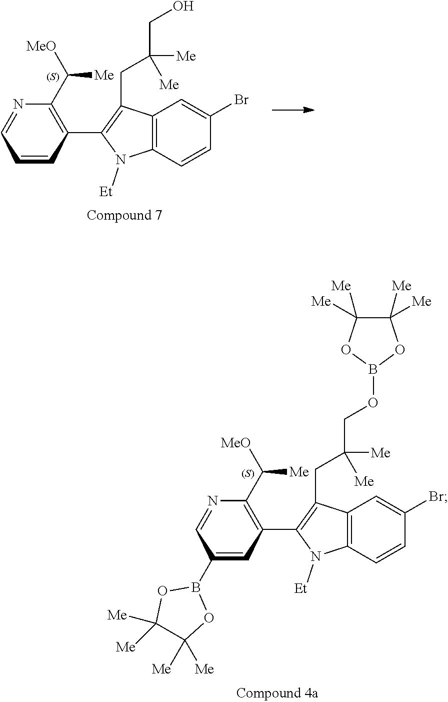

b) coupling Compound 4a and Compound 6b to form Compound 5a:

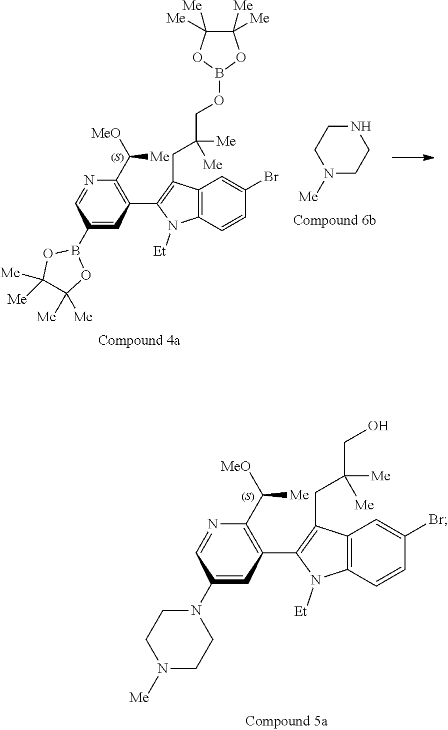

and
c) borylating Compound 5a to form Compound 6a:

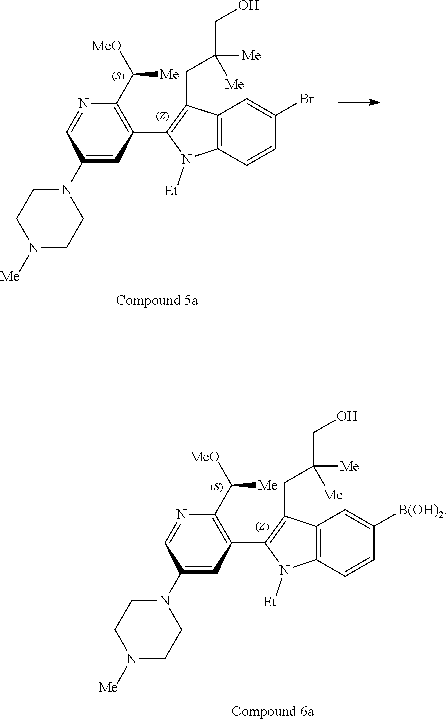

In some embodiments, the method of preparing Compound 6a includes:
a) borylating Compound 7 to form Compound 4a:

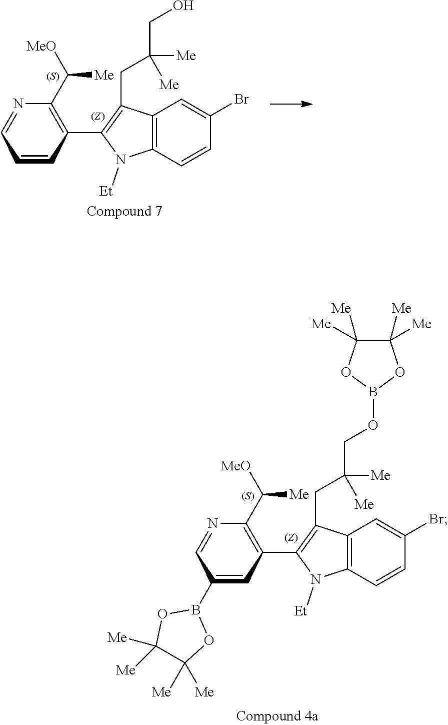

and
b) coupling Compound 4a and Compound 6b to form Compound 5a:

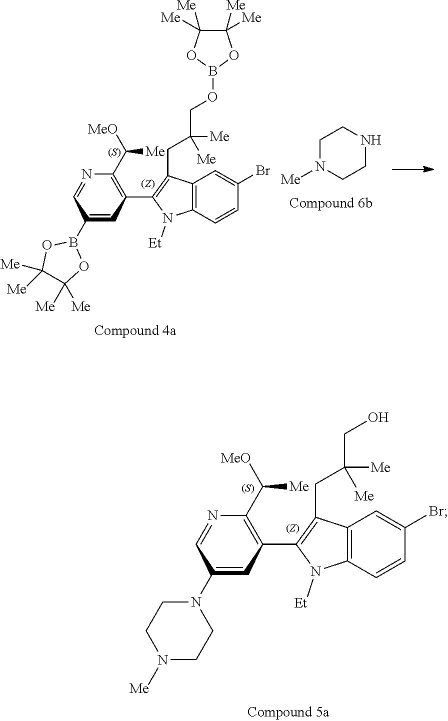

and
c) borylating Compound 5a to form Compound 6a:

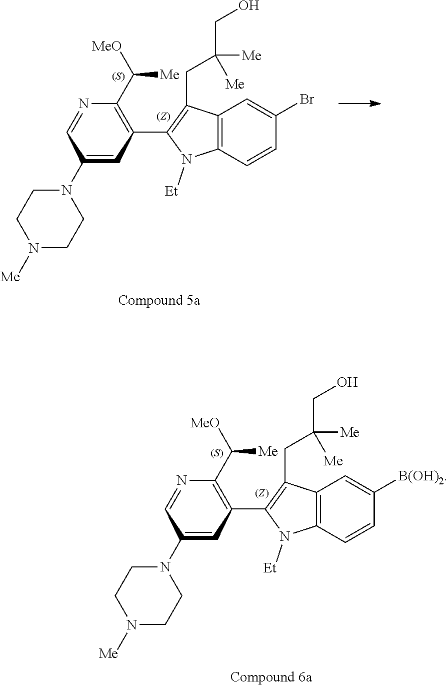

In some embodiments, the borylating step (a) includes contacting Compound 7 with an iridium catalyst. In some embodiments, the coupling step (b) includes contacting Compound 4a and Compound 5a with a copper source. In some embodiments, the copper source is Cu(OAc)₂. In some embodiments, the coupling step (b) includes a reaction in batch mode. In some embodiments, the coupling step (b) includes a reaction in flow mode. In some embodiments, the borylating step (c) includes contacting Compound 5a with a palladium catalyst and a boron source.

In another aspect, the disclosure provides a compound having the structure of Formula I:

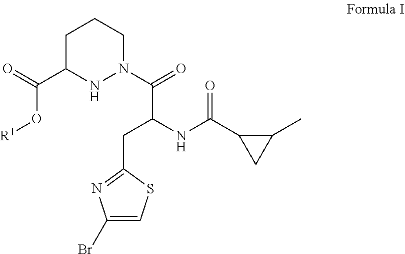

or a salt thereof, wherein R¹ is H or C₁-C₆ alkyl.

In some embodiments, the compound has the structure of Formula Ia:

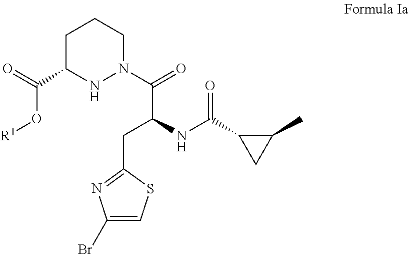

or a salt thereof.

In some embodiments, R¹ is H. In some embodiments, R¹ is CH₃.

In an aspect, the disclosure provides a method of preparing Compound 9:

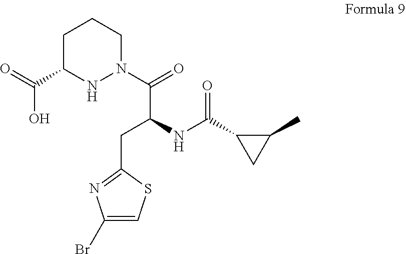 [SMILES: C[C@H]1C[C@@H]1C(=O)N[C@@H](Cc1nc(Br)cs1)C(=O)N1CCC[C@@H](C(=O)O)N1]

The method includes:
a) coupling Compound 2a with Compound 9a to form Compound 9b:

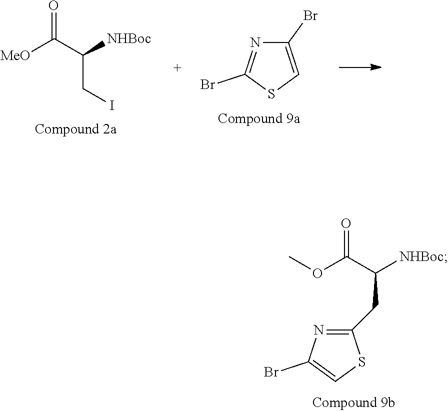 [SMILES: COC(=O)[C@H](CI)NC(=O)OC(C)(C)C ; Brc1csc(Br)n1 ; COC(=O)[C@H](Cc1nc(Br)cs1)NC(=O)OC(C)(C)C]

b) hydrolyzing Compound 9b to form Compound 9e:

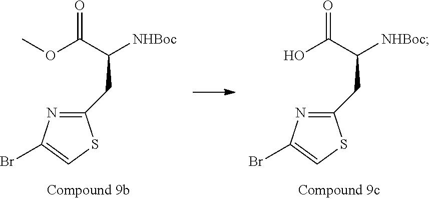 [SMILES: COC(=O)[C@H](Cc1nc(Br)cs1)NC(=O)OC(C)(C)C ; CC(C)(C)OC(=O)N[C@@H](Cc1nc(Br)cs1)C(=O)O]

c) coupling Compound 9c and Compound 9d to form Compound 9e:

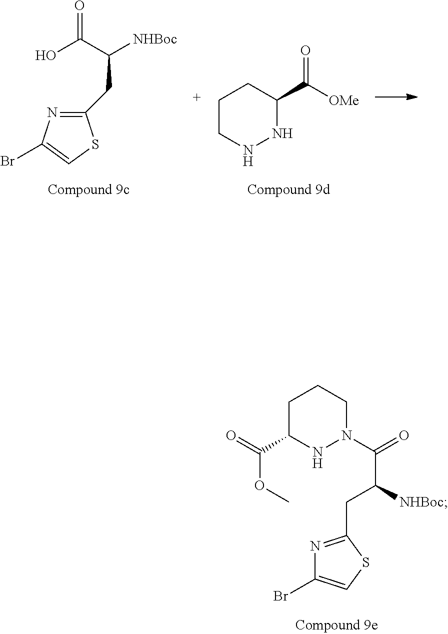 [SMILES: CC(C)(C)OC(=O)N[C@@H](Cc1nc(Br)cs1)C(=O)O ; COC(=O)[C@@H]1CCCNN1 ; COC(=O)[C@@H]1CCCN(C(=O)[C@H](Cc2nc(Br)cs2)NC(=O)OC(C)(C)C)N1]

d) deprotecting Compound 9e to form Compound 9f:

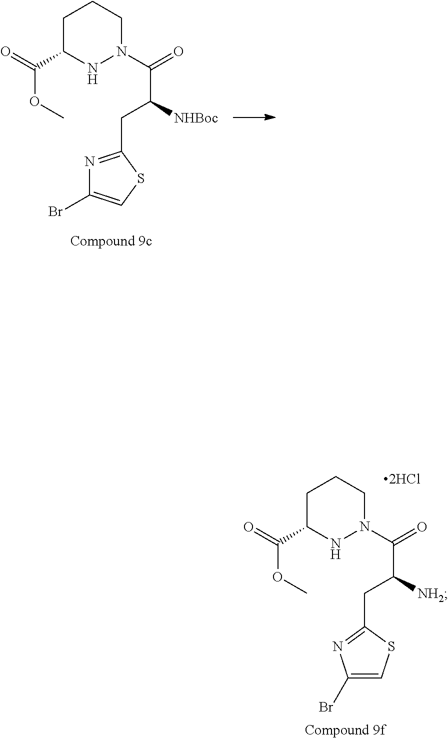 [SMILES: COC(=O)[C@@H]1CCCN(C(=O)[C@H](Cc2nc(Br)cs2)NC(=O)OC(C)(C)C)N1 ; COC(=O)[C@@H]1CCCN(C(=O)[C@@H](N)Cc2nc(Br)cs2)N1]

e) coupling Compound 9f with Compound 3 to form Compound 9g:

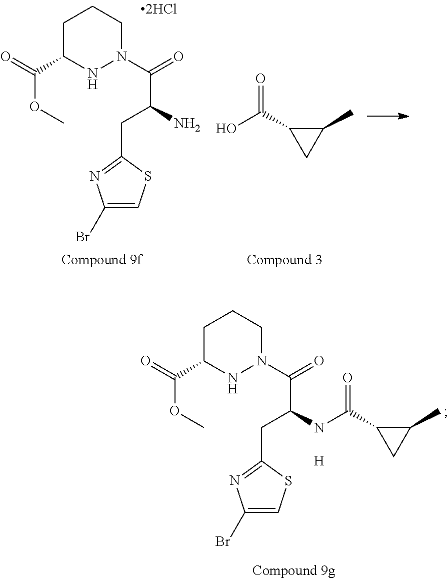

and
f) hydrolyzing Compound 9g to form Compound 9:

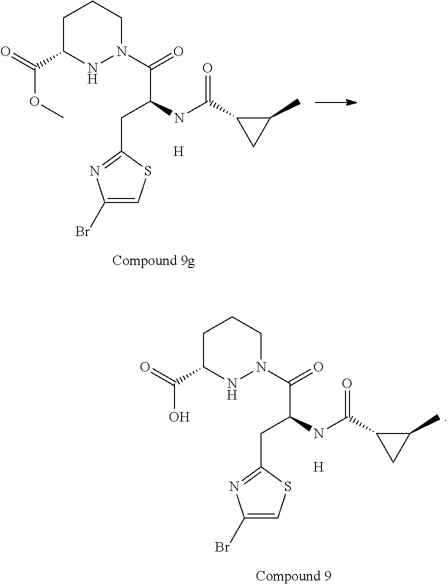

In some embodiments, the coupling step (a) includes contacting Compound 2a with a zinc source to form Compound 2a-Zn:

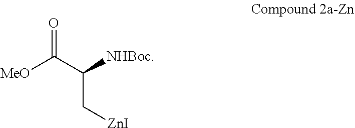

In some embodiments, coupling step (a) includes contacting Compound 2a-Zn and Compound 9a with a palladium catalyst.

In some embodiments, the coupling step (c) includes contacting Compound 9c and Compound 9d with EDCI.

In some embodiments, the coupling step (e) includes contacting Compound 9f and Compound 3 with EDCI.

In a further aspect, the disclosure provides a method of preparing Compound A. The method includes:
a) contacting Compound 6a with pinacol to form Compound 10:

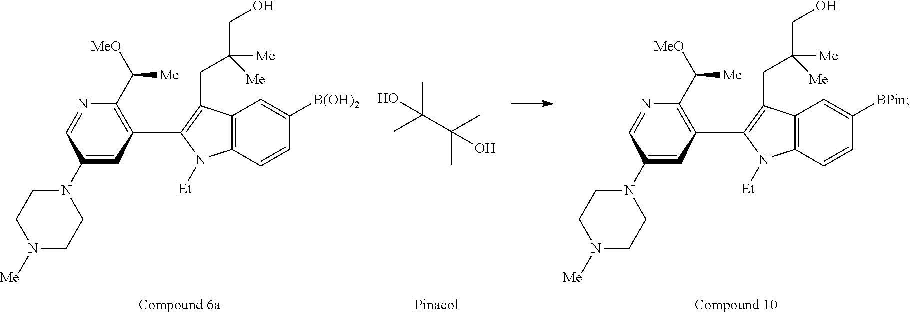

b) esterifying Compound 9 with Compound 10 to form Compound 11:

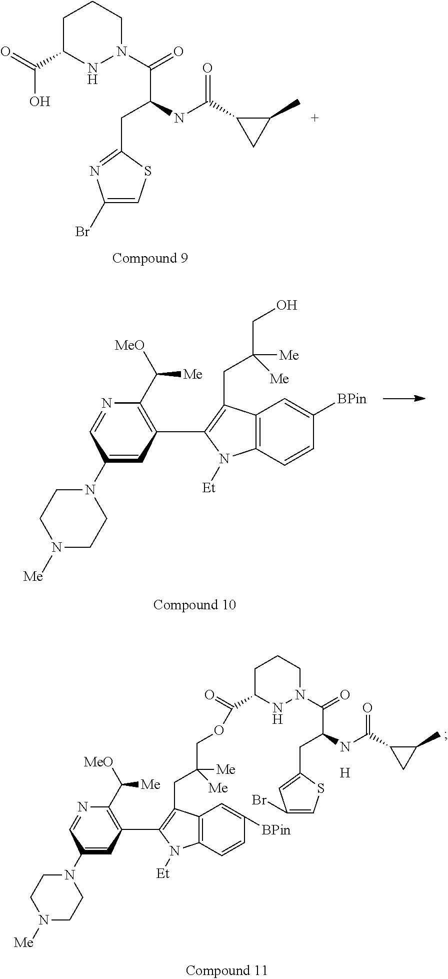

and
c) cyclizing Compound 11 to form Compound A:

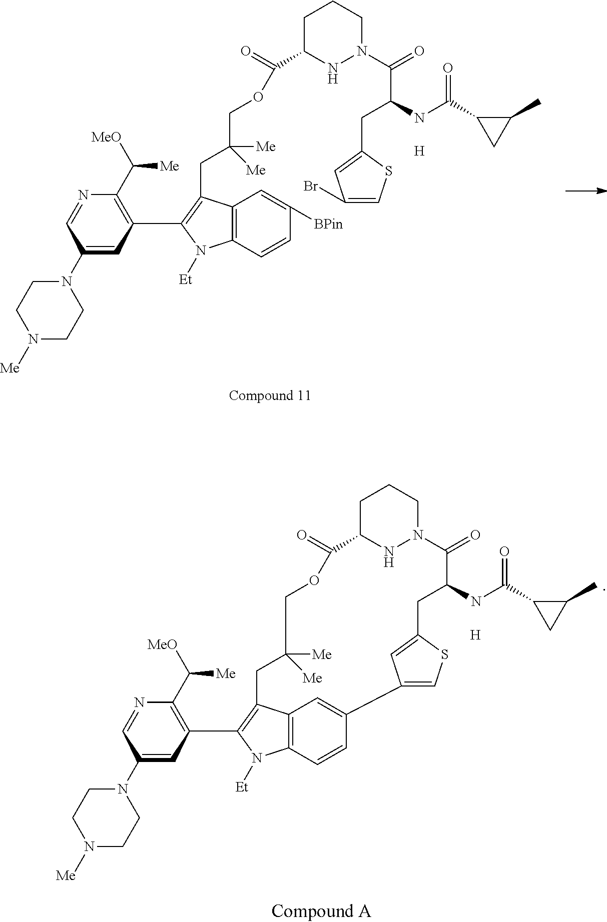

In some embodiments, the esterifying step (b) includes contacting Compound 9 and Compound 10 with EDCI. In some embodiments, the cyclizing step (c) includes contacting Compound 11 with a palladium catalyst.

In another aspect, the disclosure provides a method of preparing Compound A, the method including:
a) coupling Compound 6a and Compound 9 to form Compound 12:

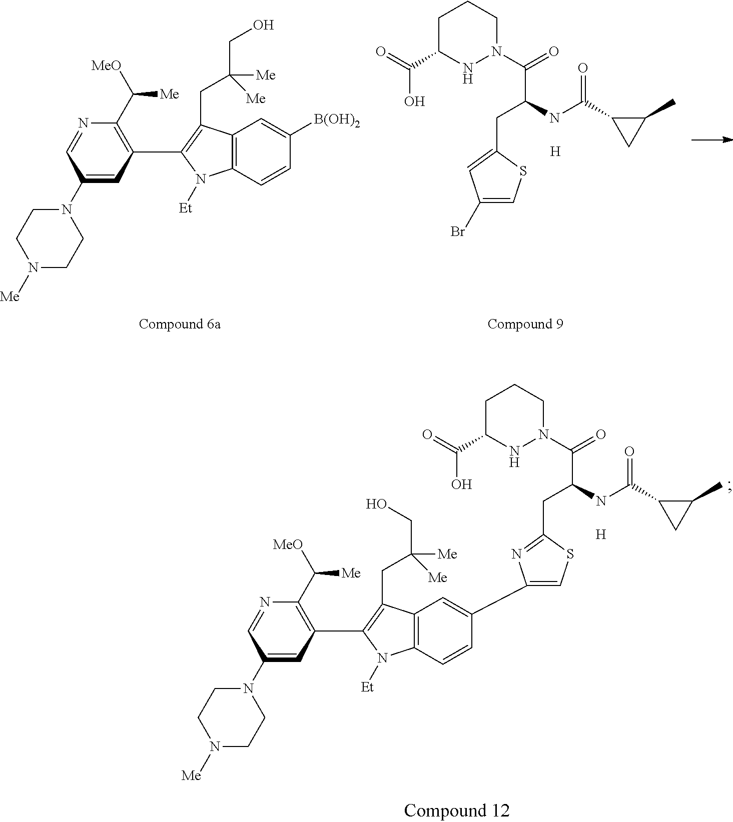

and
b) lactonizing Compound 12 to form compound A:

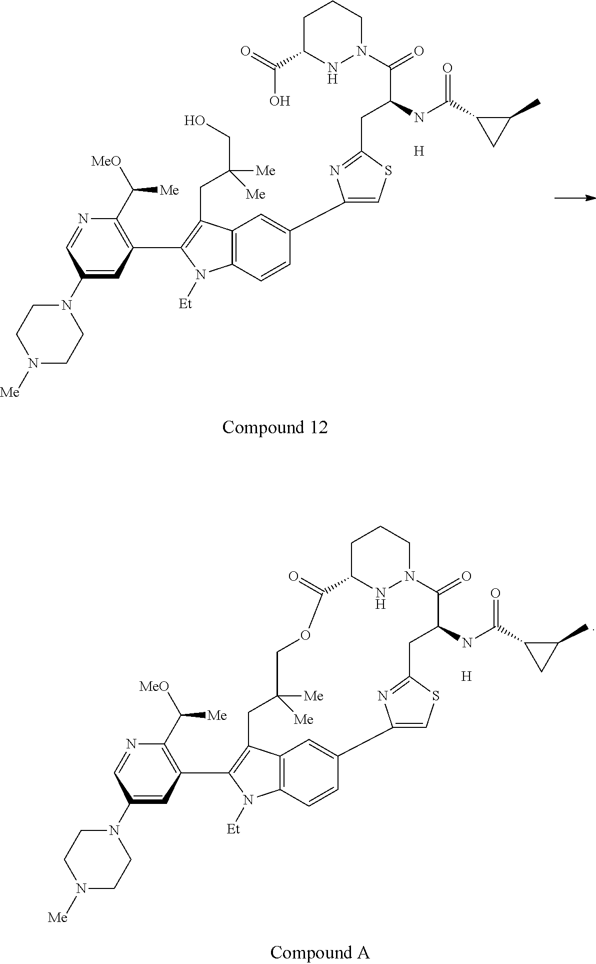

In some embodiments, the coupling step (a) includes contacting Compound 6a and Compound 9 with a palladium catalyst.

In some embodiments, the lactonizing step (b) includes contacting Compound 12 with EDCI.

In some embodiments of the methods of preparing Compound A, the method further includes purifying Compound A. In some embodiments, the purifying includes forming a salt of Compound A. In some embodiments, the salt of Compound A is a hydrochloride salt of Compound A. In some embodiments, the salt of Compound A is a lactate salt of Compound A.

In some embodiments, the purifying includes converting the salt of Compound A to a free base form of Compound A. In some embodiments, converting the salt of Compound A to a free base form of Compound A includes contacting the salt of Compound A with a base. In some embodiments, the base is sodium carbonate. In some embodiments, the method further includes precipitating the free base form of Compound A from a solution. In some embodiments, the precipitating includes adding heptane to the solution of the free base form of Compound A. In some embodiments, the free base form of Compound A is dried under humidity and nitrogen gas.

In some embodiments, the purifying includes recrystallizing Compound A. In some embodiments, the recrystallizing includes adding a first solvent followed by adding a second solvent. In some embodiments, the first solvent is a protic solvent. In some embodiments, the first solvent is methanol. In some embodiments, the second solvent is water.

In still another aspect, the disclosure provides a compound having the structure of Compound 10:

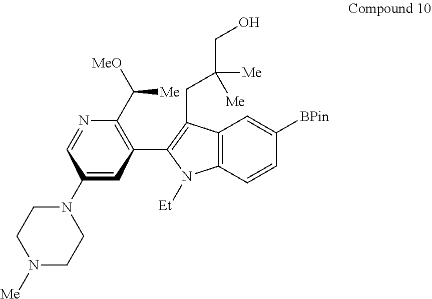

or a salt thereof.

In some embodiments, the disclosure provides a compound having the structure of Formula IV:

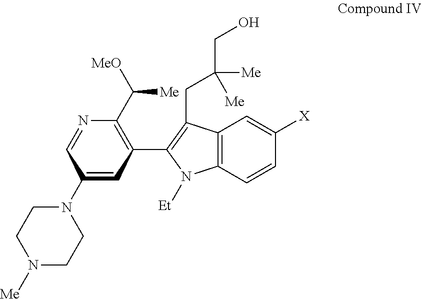

wherein X is a boronic acid, a boronate ester, or a halogen.

In a further aspect, the disclosure provides a compound having the structure of Compound 11:

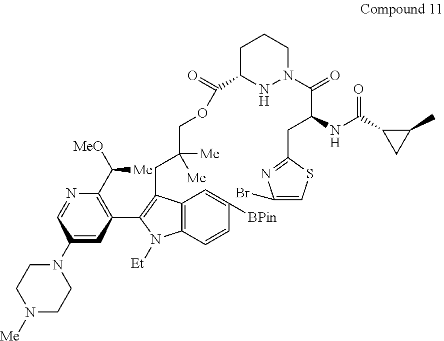

or a salt thereof.

In another aspect, the disclosure provides a compound having the structure of Compound 12:

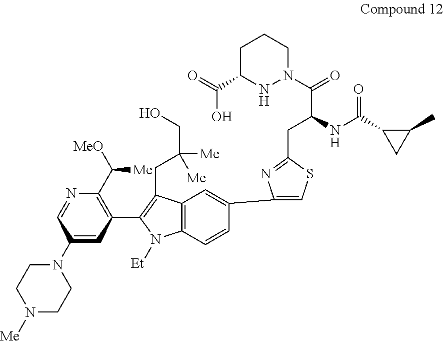

or a salt thereof.

### Definitions and Chemical Terms

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

A “compound of the present invention” and similar terms as used herein, whether explicitly noted or not, refers to Ras inhibitors described herein (e.g., Compound A) and intermediates in the synthesis thereto, as well as salts (e.g., pharmaceutically acceptable salts), solvates, hydrates, stereoisomers (including atropisomers), and tautomers thereof.

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1 H- and 3H-imidazole, 1 H-, 2H- and 4H-1,2,4-triazole, 1 H- and 2H-isoindole, and 1 H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and¹²⁵I. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein.

Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; halogen; —(CH₂)₀₋₄R^{o}; —(CH₂)₀₋₄OR^{o}; —O(CH₂)₀₋₄R^{o}; —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; 4-8 membered saturated or unsaturated heterocycloalkyl (e.g., pyridyl); 3-8 membered saturated or unsaturated cycloalkyl (e.g., cyclopropyl, cyclobutyl, or cyclopentyl); —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}2; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄—C(O)—N(R^{o})₂; —(CH₂)₀₋₄—C(O)—N(R^{o})—S(O)₂—R^{o}; —C(NCN)NR^{o}₂; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}; —SC(S)SR^{o}; —(CH₂)₀₋₄SC(O)R^{o}; —(CH₂)₀₋₄C(O)NR^{o}2; —C(S)NR^{o}₂; —C(S)SR^{o}; —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}2; —N(R^{o})S(O)₂R^{o}; —N(OR^{o})R^{o}; —C(NOR^{o})NR^{o}₂; —C(NH)NR^{o}₂; —P(O)₂R^{o}; —P(O)R^{o}₂; —P(O)(OR^{o})₂; —OP(O)R^{o}₂; —OP(O)(OR^{o})₂; —OP(O)(OR^{o})R^{o}, —SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, -C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), may be, independently, halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, -(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{\}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “amino,” as used herein, represents —N(R^{†})₂, e.g., —NH₂ and —N(CH₃)₂.

The term “aminoalkyl,” as used herein, represents an alkyl moiety substituted on one or more carbon atoms with one or more amino moieties.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term —N(C(O)—(C₀-C₅ alkylene-H)— includes —N(C(O)—(C₀ alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “cyano,” as used herein, represents a —CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatomn may appear in the middle or at the end of the radical.

The term “heteroaryl,” as used herein, represents a monovalent, monocyclic, or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiments, the heteroaryl is substituted with 1, 2, 3, or 4 substituents groups.

The term “heterocycloalkyl,” as used herein, represents a monovalent monocyclic, bicyclic, or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more —OH moieties.

The term “isomer,” as used herein, means any tautomer, stereoisomer, atropiosmer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, recrystallizing the compound as a chiral salt complex, or recrystallizing the compound in a chiral solvent. Enantiomers and stereoisomers can also be obtained from stereomerically or enantiomerically pure intermediates, reagents, and catalysts by well-known asymmetric synthetic methods.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

The term “thiocarbonyl,” as used herein, refers to a —C(S)— group.

The term “Boc” refers to a tert-butyloxycarbonyl or tert-butoxycarbonyl protecting group having the structure

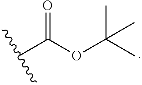 [SMILES: *C(=O)OC(C)(C)C]

The term “BPin” refers to a pinacolborane group having the structure:

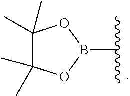 [SMILES: *B1OC(C)(C)C(C)(C)O1]

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

### DETAILED DESCRIPTION

Provided herein are synthetic methods and intermediates for making the Ras inhibitor Compound A, or a salt thereof. The methods and intermediates can be useful for achieving a higher yield, a higher chemical purity, and/or a higher stereoisomeric purity, and a lower cost for the preparation of Compound A. Further synthetic details are provided in the Examples. The structure of Compound A is shown below.

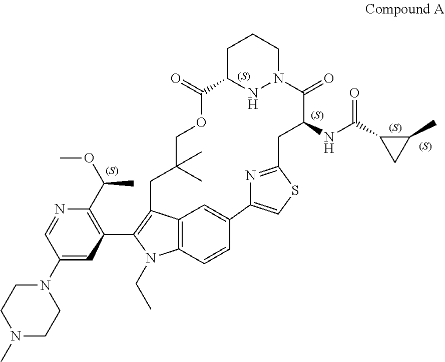

The compounds described herein may be prepared using the methods described herein and/or using known organic, inorganic, or enzymatic processes. The synthetic methods may employ the use of commercially available starting materials or starting materials prepared by processes known to those skilled in the art of organic synthesis. These methods include but are not limited to those methods described in the Schemes below and in WO 2021/091967 and WO 2022/060836, the disclosure of each of which is incorporated herein by reference.

In one aspect, the disclosure provides a method of preparing Compound 1:

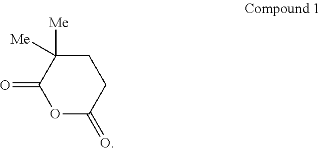

The method of preparing may include
a) reacting Compound 1a and Compound 1 b to form Compound 1c:

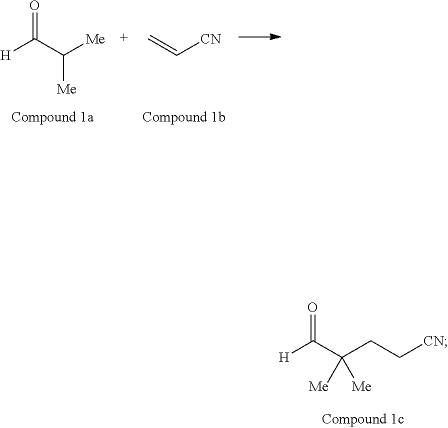

b) oxidizing and hydrolyzing Compound 1c to form Compound 1d:

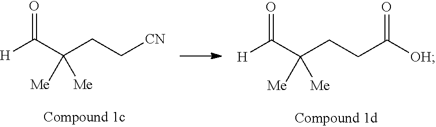 [SMILES: CC(C)(C=O)CCC#N ; CC(C)(C=O)CCC(=O)O]

and
c) cyclizing Compound 1d to form Compound 1:

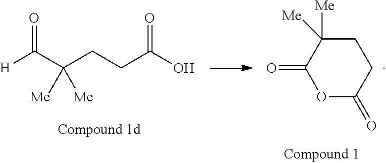 [SMILES: CC(C)(C=O)CCC(=O)O ; CC1(C)CCC(=O)OC1=O]

In some embodiments, the reacting step (a) includes contacting Compound 1a and Compound 1b with a base. In some embodiments, the base is sodium hydroxide. In some embodiments, an excess of Compound 1b is used relative to the amount of Compound 1a. In some embodiments, 1.25 equivalents of Compound 1b is used relative to Compound 1a. In some embodiments, the reacting step (a) is carried out in the presence of hydroquinone. In some embodiments, less than 1 equivalent (e.g., less than 0.9 equivalents, less than 0.5 equivalents, less than 0.25 equivalents, less than 0.1 equivalents, or less than 0.01 equivalents) of hydroquinone is used relative to the amount of Compound 1a. In some embodiments, less than 0.01 equivalents of hydroquinone are used relative to the amount of Compound 1a. In some embodiments, the reacting step (a) is carried out in a solvent. In some embodiments, the solvent is an ethereal solvent. In some embodiments, the solvent is a dioxane. In some embodiments, the solvent is 1,4-dioxane. In some embodiments, the reacting step (a) is carried out above room temperature (e.g., above 20° C., above 30° C., above 40° C., above 50° C., above 60° C., or above 70° C.). In some embodiments, the reacting step (a) is carried out at 65° C. In some embodiments, the reacting step (a) is carried out between 70 to 75° C.

In some embodiments, the reacting step (a) is carried out according to the following scheme:

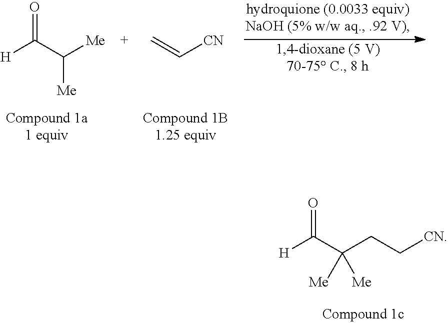

In some embodiments, the reacting step (a) is carried out according to the following scheme:

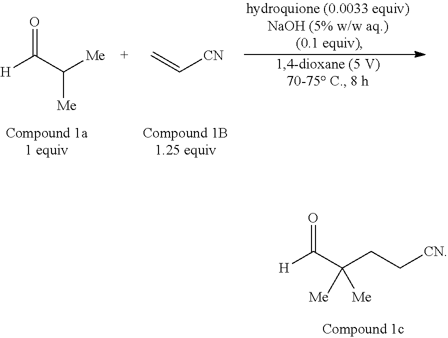

In some embodiments, the oxidizing and hydrolyzing step (b) is carried out in the presence of sulfuric acid and nitric acid. In some embodiments, the reaction in the presence of sulfuric acid and nitric acid is carried out above room temperature (e.g., above 20° C., above 30° C., above 40° C., above 50° C., above 60° C., or above 70° C.). In some embodiments, the reaction in the presence of sulfuric acid and nitric acid is carried out between 70 to 75° C.

In some embodiments, the oxidizing and hydrolyzing step (b) is carried out according to the following scheme:

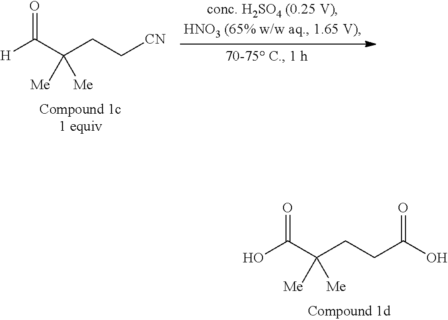 [SMILES: CC(C)(C=O)CCC#N ; CC(C)(CCC(=O)O)C(=O)O]

In some embodiments, the oxidizing and hydrolyzing step (b) includes a first step of oxidizing Compound 1c to Compound 1e and a second step of hydrolyzing Compound 1e to Compound 1d:

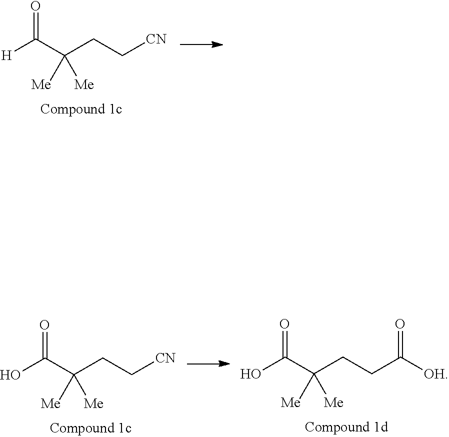 [SMILES: CC(C)(C=O)CCC#N ; CC(C)(CCC#N)C(=O)O ; CC(C)(CCC(=O)O)C(=O)O]

In some embodiments, the oxidizing step includes contacting NaClO₂ and Compound 1c. In some embodiments, the oxidizing step includes contacting Compound 1c with NaClO₂ and KH₂PO₄. In some embodiments, the oxidizing step includes contacting Compound 1c with NaClO₂, KH₂PO₄, and DMSO. In some embodiments, the oxidizing step is carried out in a solvent. In some embodiments, the solvent is water.

In some embodiments, the hydrolyzing step includes contacting potassium hydroxide and Compound 1e. In some embodiments, Compound 1e is contacted with more than one equivalent (e.g., more than three equivalents) of potassium hydroxide. In some embodiments, Compound 1e is contacted with potassium hydroxide above 30° C. (e.g., above 40° C., above 50° C., above 60° C., above 70° C., above 80° C., or above 90° C.). In some embodiments, Compound 1e is contacted with potassium hydroxide between 9° and 100° C. In some embodiments, the second step further includes protonating Compound 1d by contacting the reaction with hydrochloric acid.

In some embodiments of the method of preparing Compound 1, the cyclizing step (c) includes contacting acetic anhydride and Compound 1d. In some embodiments, the cyclizing step (c) includes contacting Compound 1d with acetic anhydride for at least one hour. In some embodiments, the cyclizing step (c) includes contacting Compound 1d with acetic anhydride at a temperature above 80° C. (e.g., between 8° and 85° C. or at 110° C.).

In some embodiments, the oxidizing and hydrolyzing step (b) and the cyclizing step (c) are carried out according to the following scheme:

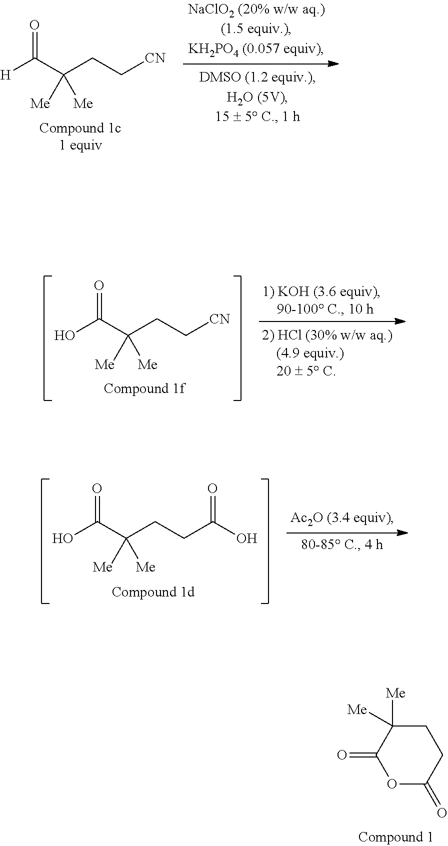

In some embodiments, the method further includes purifying Compound 1 by decolorization with activated carbon. In some embodiments, Compound 1 is purified by recrystallization. In some embodiments, the recrystallization is repeated more than once. In some embodiments, the recrystallization is carried out in methyl tert-butyl ether and n-hexane.

In a further aspect, the disclosure provides a method of preparing Compound 2a. The method includes:
a) esterifying Compound 2b to form Compound 2c:

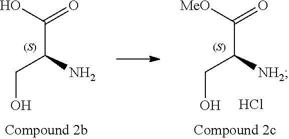

b) protecting and tosylating Compound 2c to form Compound 2d:

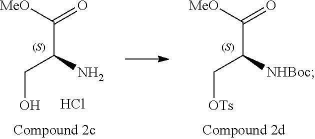

and
c) iodinating Compound 2d to form Compound 2a:

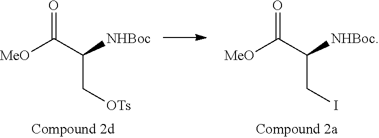 [SMILES: COC(=O)[C@H](COS(=O)(=O)c1ccc(C)cc1)NC(=O)OC(C)(C)C ; COC(=O)[C@H](CI)NC(=O)OC(C)(C)C]

In some embodiments of the method of preparing Compound 2a, the esterifying step (a) includes contacting a methanol solution of thionyl chloride and Compound 2b. In some embodiments, Compound 2b is contacted with a methanol solution of thionyl chloride at room temperature (e.g., between 15 and 25° C., at 20° C., or at 25° C.).

In some embodiments, the protecting and tosylating step (b) includes a first step of protecting Compound 2c to form Compound 2e and a second step of tosylating Compound 2e to form Compound 2d:

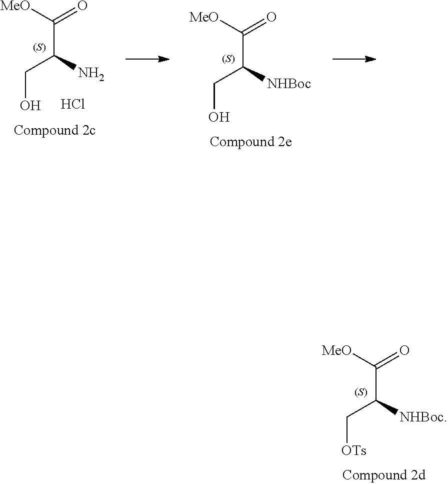

In some embodiments, the first protecting step includes contacting di-tert-butyl dicarbonate and Compound 2c, and the second tosylating step includes contacting tosyl chloride and Compound 2e. In some embodiments, the first protecting step further includes contacting Compound 2c with a base. In some embodiments, the base is sodium bicarbonate. In some embodiments, the second tosylating step further includes contacting Compound 2e with a base. In some embodiments, the base is pyridine.

In some embodiments, the iodinating step (c) includes contacting compound 2d with sodium iodide. In some embodiments, the iodinating step (c) further includes contacting compound 2d with an acid. In some embodiments, the acid is citric acid.

In some embodiments, the method of preparing Compound 2a is carried out according to the following scheme:

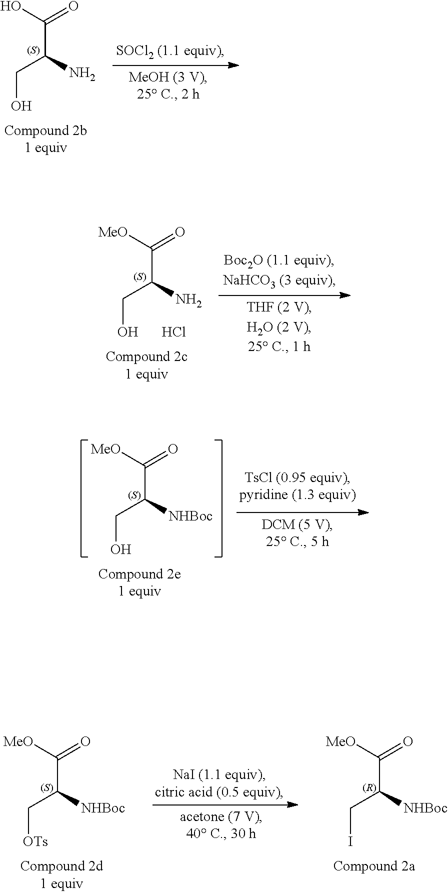

In an aspect, the disclosure provides a method of preparing Compound 3:

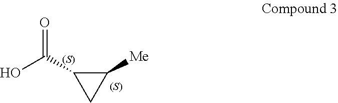

The method includes:
a) contacting Compound 3a and Compound 3b in the presence of a base to form Compound

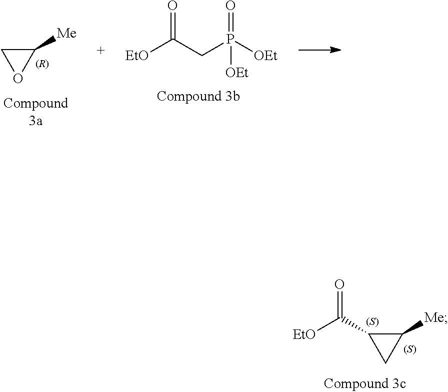

and
b) hydrolyzing Compound 3c to form Compound 3:

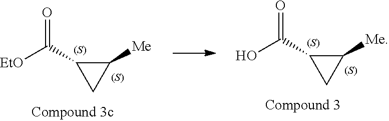

In some embodiments, the base of step (a) is n-butyllithium. In some embodiments, the contacting step (a) is carried out using a flow process. In some embodiments, the hydrolyzing step (b) includes contacting Compound 3c with a hydroxide base. In some embodiments, the hydroxide base is sodium hydroxide. In some embodiments, the hydrolyzing step (b) further includes contacting Compound 3c with dicyclohexylamine. In some embodiments, the hydrolyzing step (b) first forms a dicyclohexylamine salt of Compound 3. In some embodiments, the hydrolyzing step (b) further includes contacting dicyclohexylamine salt of Compound 3 with (R)-(+)-N-benzyl-1-phenylethylamine.

In some embodiments, the contacting step (a) is carried out according to the following scheme:

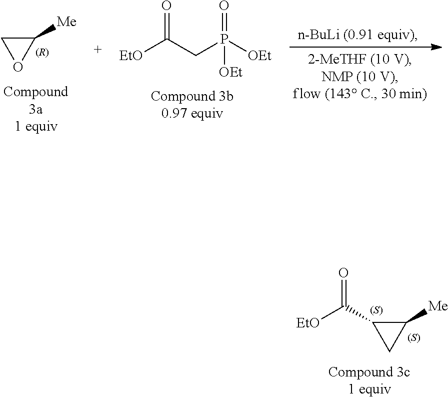

In some embodiments, the hydrolyzing step (b) is carried out according to the following scheme:

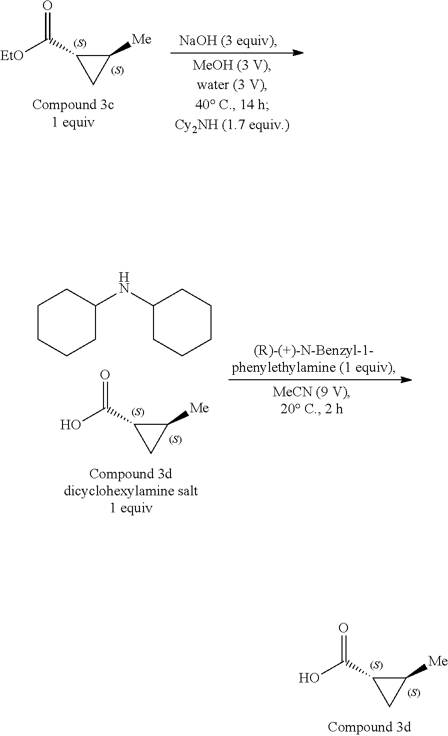

In some embodiments, disclosure provides a method of preparing Compound 6a. The method includes:
a) borylating Compound 7 to form Compound 4a:

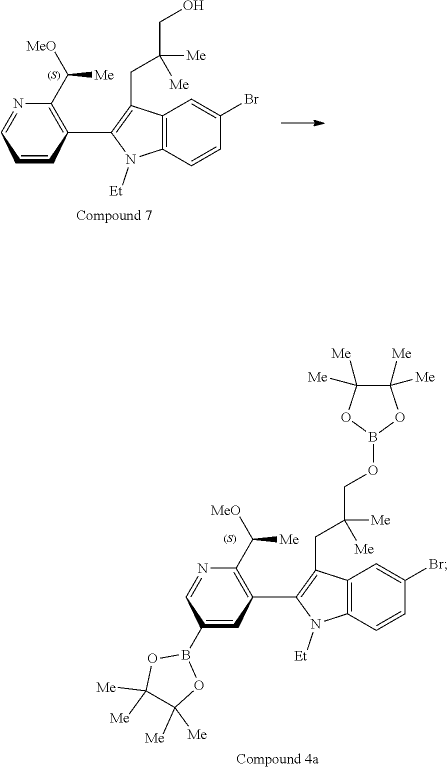

b) coupling Compound 4a and Compound 6b to form Compound 5a:

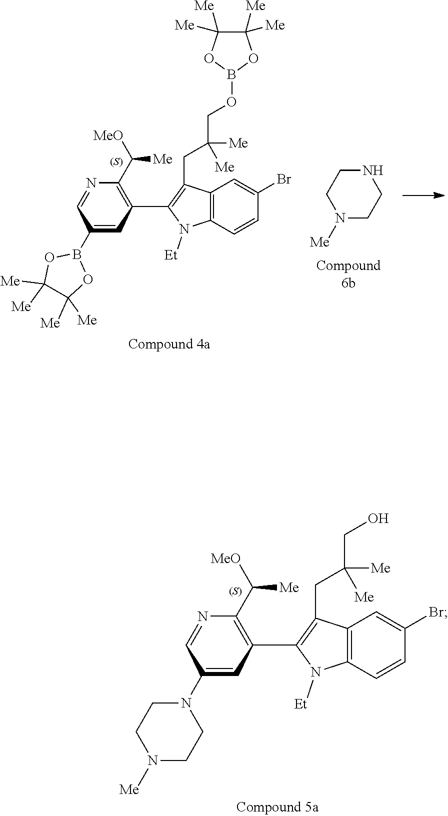

and
c) borylating Compound 5a to form Compound 6a:

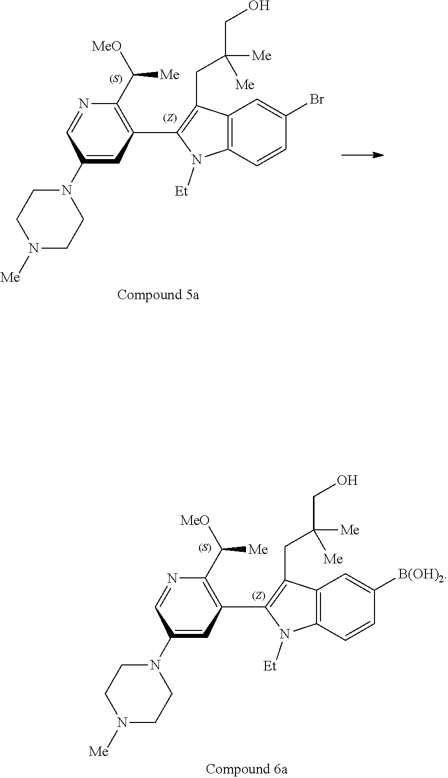

In some embodiments, the borylating step (a) includes contacting Compound 7 with an iridium catalyst. In some embodiments, the borylating step (a) further includes contacting Compound 7 with bis-(pinacolato)diboron. In some embodiments, the borylating step (b) is carried out according to the following scheme:

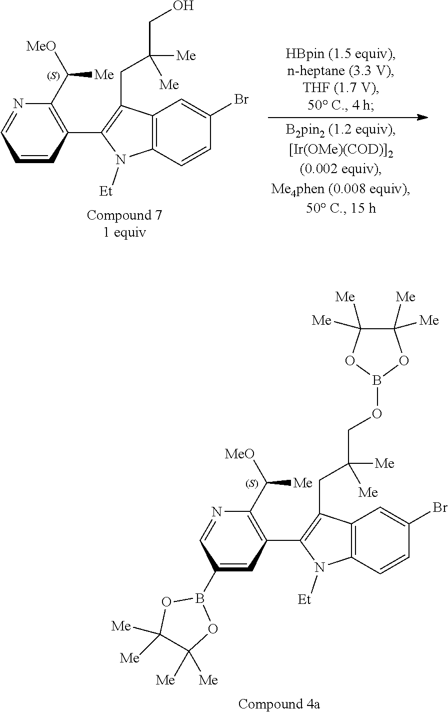

In some embodiments, the coupling step (b) includes contacting Compound 4a and Compound 6b with a copper source. In some embodiments, the copper source is Cu(OAc)₂. In some embodiments, the coupling step (b) includes a reaction in batch mode. In some embodiments, the coupling step (b) includes a reaction in flow mode. In some embodiments, the coupling step (b) further includes contacting Compound 4a and Compound 6b with oxygen gas.

In some embodiments, the coupling step (b) is carried out according to the following scheme:

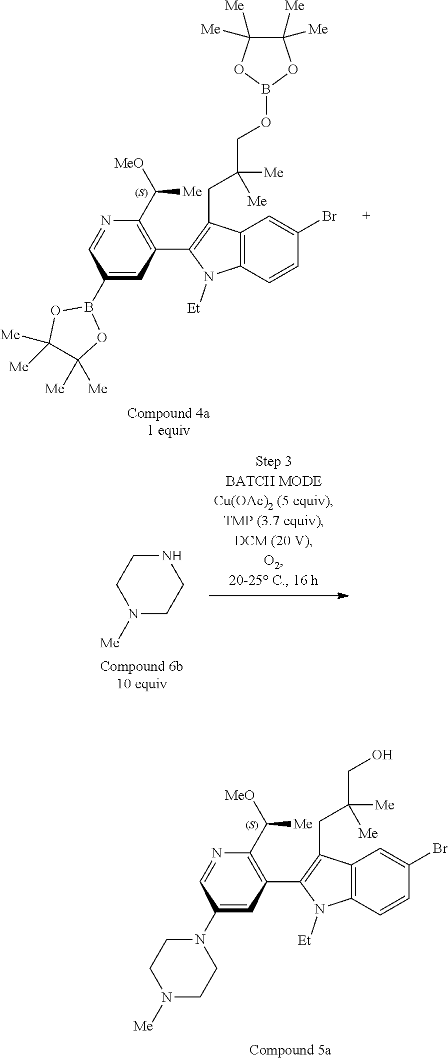

In some embodiments, the borylating step (c) includes contacting Compound 5a with a palladium catalyst and a boron source. In some embodiments, the boron source is B₂(OH)₄. In some embodiments, the borylating step (c) is carried out according to the following scheme:

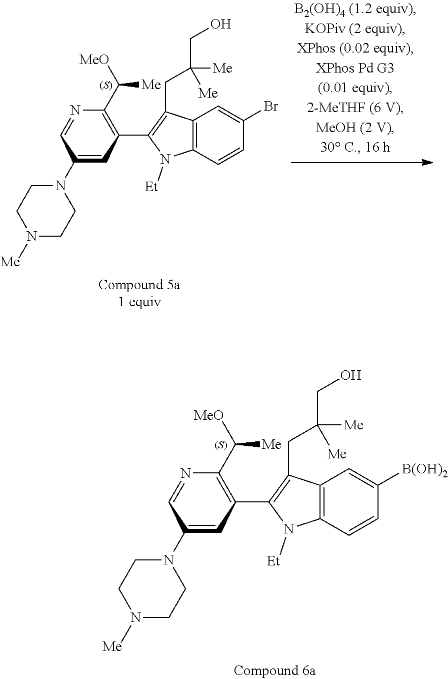

In some embodiments of the above reaction, 1.5 eq of B₂(OH)₄ is used. In some embodiments of the above reaction, 2.1 eq of KOPiv is used.

In some embodiments, the disclosure provides a method of preparing Compound 9:

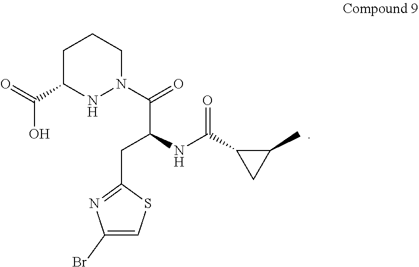 [SMILES: C[C@H]1C[C@@H]1C(=O)N[C@@H](Cc1nc(Br)cs1)C(=O)N1CCC[C@@H](C(=O)O)N1]

The method includes:
a) coupling Compound 2a with Compound 9a to form Compound 9b:

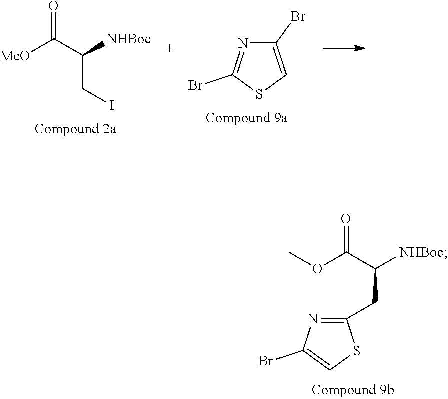 [SMILES: COC(=O)[C@H](CI)NC(=O)OC(C)(C)C ; Brc1csc(Br)n1 ; COC(=O)[C@H](Cc1nc(Br)cs1)NC(=O)OC(C)(C)C]

b) hydrolyzing Compound 9b to form Compound 9c:

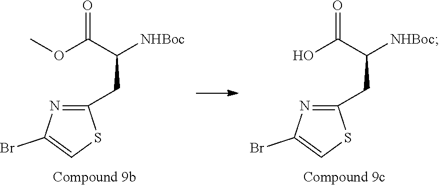 [SMILES: COC(=O)[C@H](Cc1nc(Br)cs1)NC(=O)OC(C)(C)C ; CC(C)(C)OC(=O)N[C@@H](Cc1nc(Br)cs1)C(=O)O]

c) coupling Compound 9c and Compound 9d to form Compound 9e:

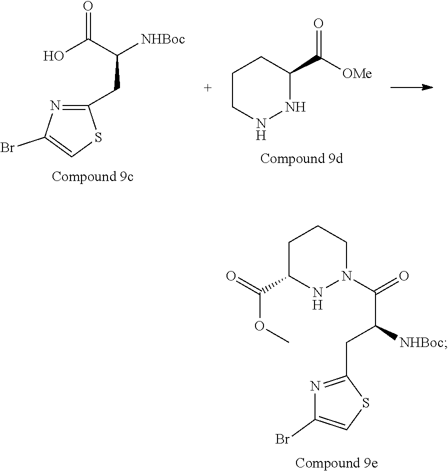 [SMILES: CC(C)(C)OC(=O)N[C@@H](Cc1nc(Br)cs1)C(=O)O ; COC(=O)[C@@H]1CCCNN1 ; COC(=O)[C@@H]1CCCN(C(=O)[C@H](Cc2nc(Br)cs2)NC(=O)OC(C)(C)C)N1]

d) deprotecting Compound 9e to form Compound 9f:

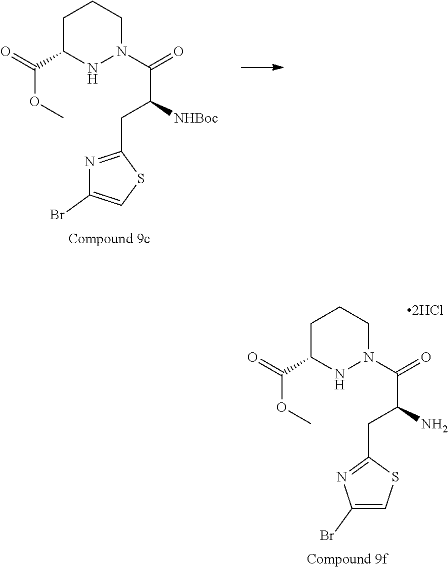 [SMILES: COC(=O)[C@@H]1CCCN(C(=O)[C@H](Cc2nc(Br)cs2)NC(=O)OC(C)(C)C)N1 ; COC(=O)[C@@H]1CCCN(C(=O)[C@@H](N)Cc2nc(Br)cs2)N1]

e) coupling Compound 9f with Compound 3 to form Compound 9g:

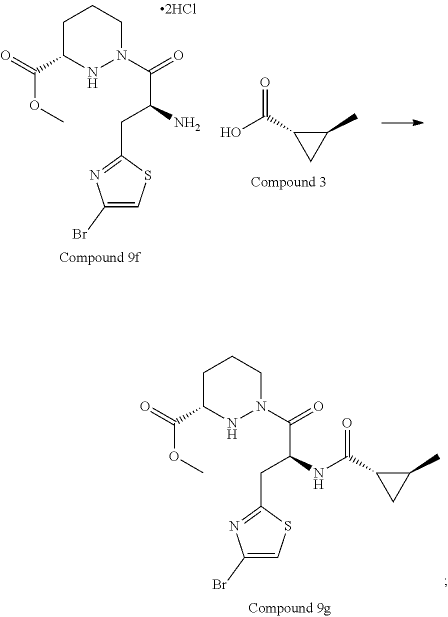

and
f) hydrolyzing Compound 9g to form Compound 9:

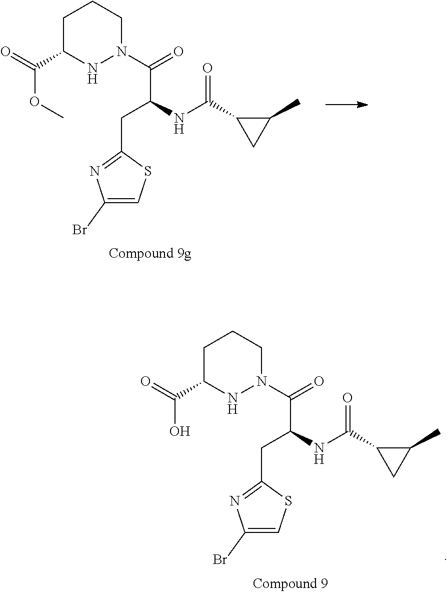 [SMILES: COC(=O)[C@@H]1CCCN(C(=O)[C@H](Cc2nc(Br)cs2)NC(=O)[C@H]2C[C@@H]2C)N1 ; C[C@H]1C[C@@H]1C(=O)N[C@@H](Cc1nc(Br)cs1)C(=O)N1CCC[C@@H](C(=O)O)N1]

In some embodiments, the disclosure provides a method of preparing Compound 9c:

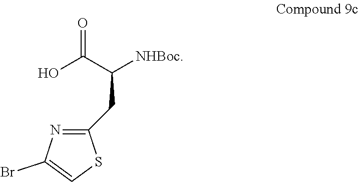 [SMILES: CC(C)(C)OC(=O)N[C@@H](Cc1nc(Br)cs1)C(=O)O]

or a salt thereof.
The method includes:
a) formylating Compound 9c-1 to form Compound 9c-2:

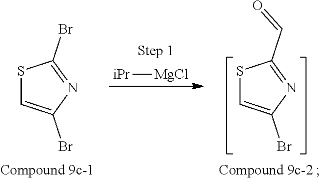

b) condensing Compound 9c with malonic acid to form Compound 9c-3:

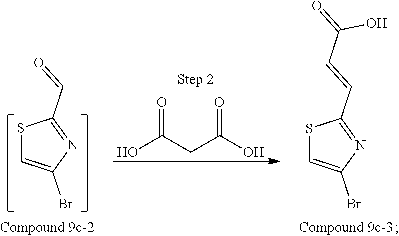

c) aminating Compound 9c-3 to form Compound 9c-4 H₂O:

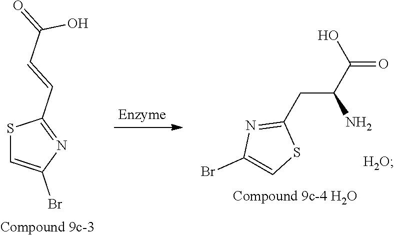

and
d) protecting Compound 9-4 to form Compound 9c:

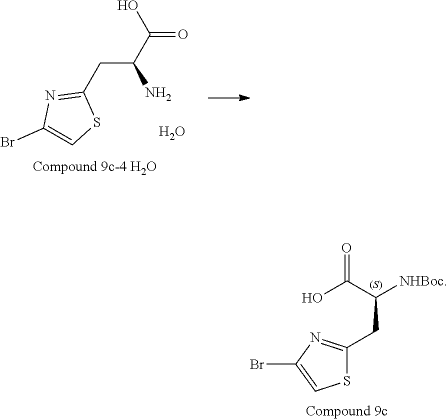

In some embodiments, the aminating step c) is carried out using an enzyme. In some embodiments, the enzyme is a phenylalanine ammonia lyase (PAL). PALs are well-known in the art and can be sourced from a variety of suppliers, such as Pharmaron (e.g., PH-AML-18), as well as Hande, Apeloa and WuXi STA. In some embodiments, Compound 9c prepared by this method can be used in a method of preparing Compound 9.

In some embodiments, Compound 9c synthesized according to the following scheme:

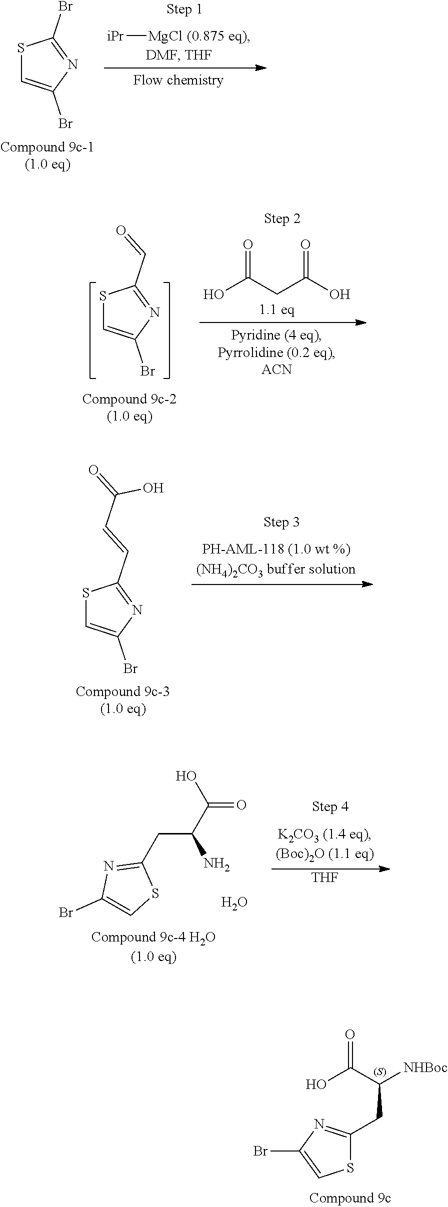

In some embodiments, the coupling step (a) includes contacting Compound 2a with a zinc source to form Compound 2a-Zn:

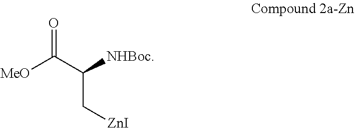

In some embodiments, the contacting Compound 2a with a zinc source further includes contacting 2a with 1,2-dibromoethane. In some embodiments, the contacting 2a with a zinc source further includes contacting Compound 2a with trimethylsilyl chloride.

In some embodiments, coupling step (a) includes contacting Compound 2a-Zn and Compound 9a with a palladium catalyst. In some embodiments, the coupling step (a) is carried out according to the following scheme:

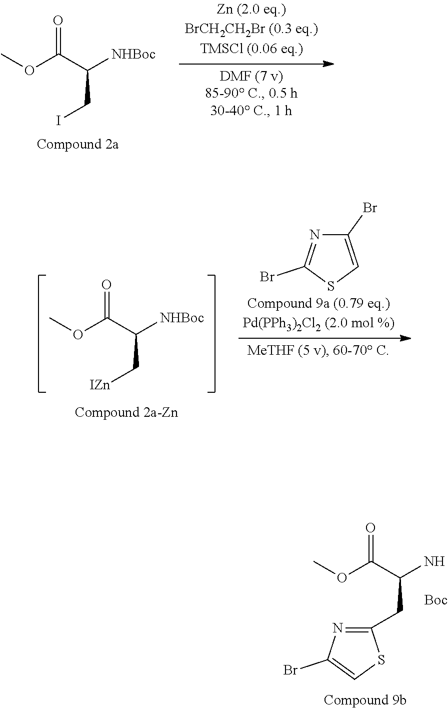

In some embodiments, the hydrolyzing step (b) is carried out according to the following scheme:

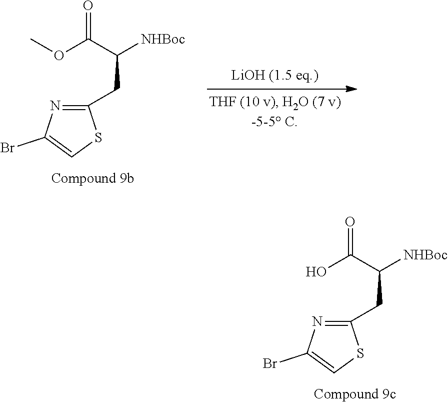 [SMILES: COC(=O)[C@H](Cc1nc(Br)cs1)NC(=O)OC(C)(C)C ; CC(C)(C)OC(=O)N[C@@H](Cc1nc(Br)cs1)C(=O)O]

In some embodiments, the coupling step (c) includes contacting Compound 9c and Compound 9d with EDCI. In some embodiments, the coupling step (c) further includes contacting Compound 9c and Compound 9d with HOBt. In some embodiments, the coupling step (c) is carried out according to the following scheme:

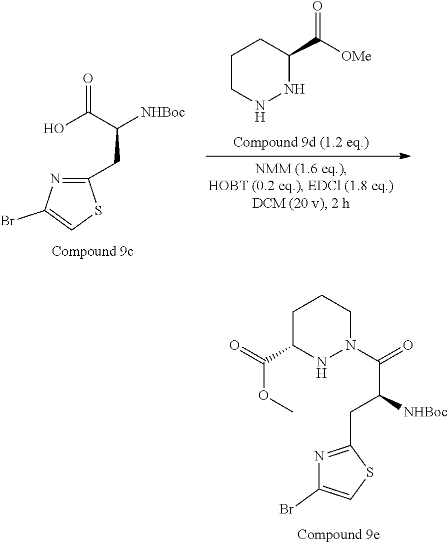 [SMILES: CC(C)(C)OC(=O)N[C@@H](Cc1nc(Br)cs1)C(=O)O ; COC(=O)[C@@H]1CCCNN1 ; COC(=O)[C@@H]1CCCN(C(=O)[C@H](Cc2nc(Br)cs2)NC(=O)OC(C)(C)C)N1]

In some embodiments, the deprotecting step (d) includes contacting Compound 9e with thionyl chloride. In some embodiments, the deprotecting step (d) is carried out according to the following scheme:

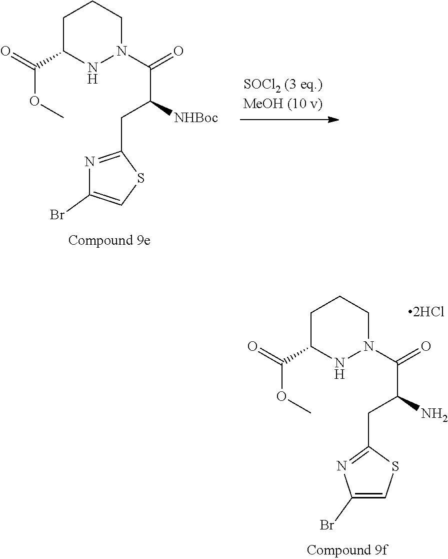 [SMILES: COC(=O)[C@@H]1CCCN(C(=O)[C@H](Cc2nc(Br)cs2)NC(=O)OC(C)(C)C)N1 ; COC(=O)[C@@H]1CCCN(C(=O)[C@@H](N)Cc2nc(Br)cs2)N1]

In some embodiments, the coupling step (e) includes contacting Compound 9f and Compound 3 with EDCI. In some embodiments, the coupling step (e) further includes contacting Compound 9f and Compound 3 with NMM. In some embodiments, the coupling step (e) further includes contacting Compound 9f and Compound 3 with HOBt.

In some embodiments, the coupling step (e) is carried out according to the following scheme:

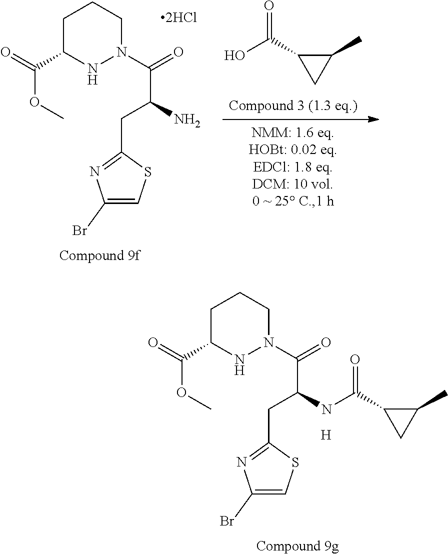

In some embodiments, the hydrolyzing step (f) is carried out according to the following scheme:

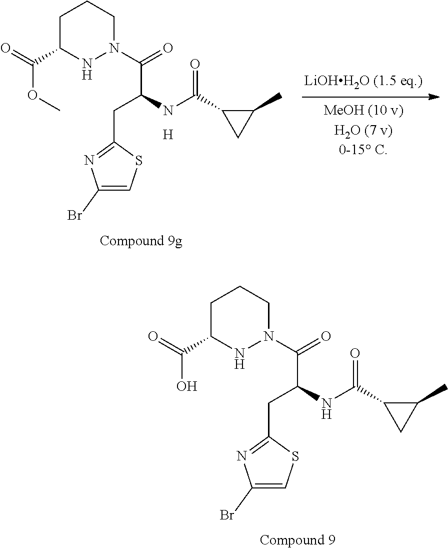

In a further aspect, the disclosure provides a method of preparing Compound A. The method includes:
a) contacting Compound 6a with pinacol to form Compound 10:

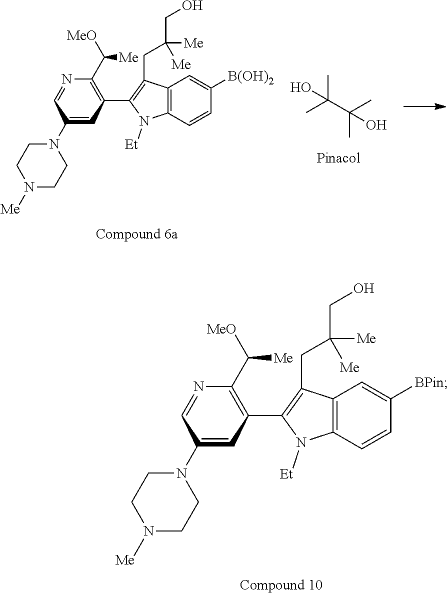

b) esterifying Compound 9 with Compound 10 to form Compound 11:

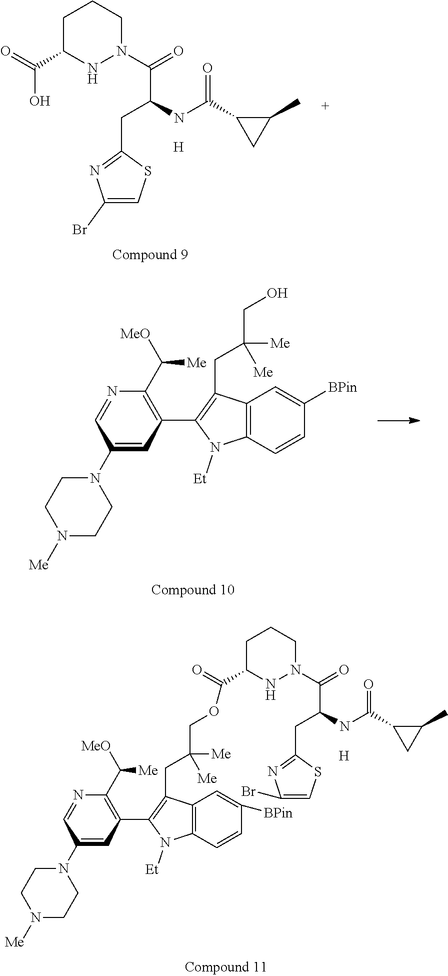

and
c) cyclizing Compound 11 to form Compound A:

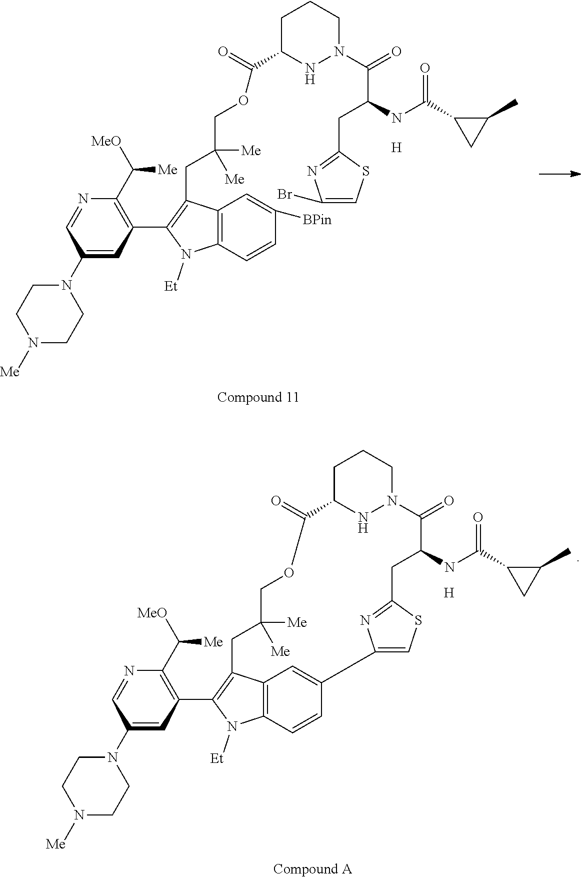

In some embodiments, the esterifying step (b) includes contacting Compound 9 and Compound 10 with EDCI. In some embodiments, the esterifying step (b) further includes contacting Compound 9 and Compound 10 with a base (e.g., DMAP).

In some embodiments, the esterifying step (e) is carried out according to the following scheme:

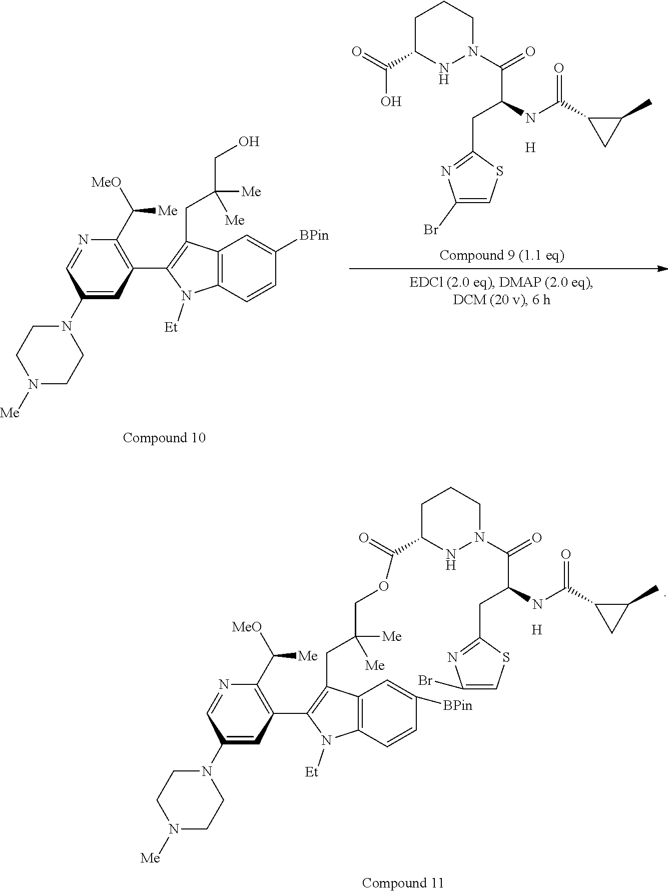

In some embodiments, the cyclizing step (c) includes contacting Compound 11 with a palladium catalyst.

In some embodiments, the cyclizing step (c) is carried out according to the following scheme:

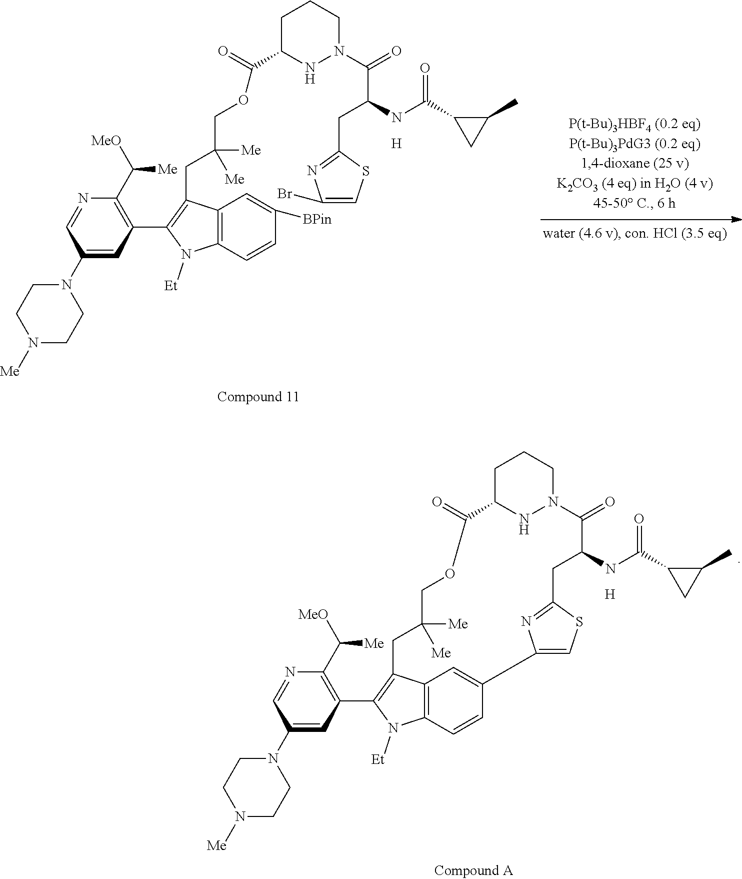

In another aspect, the disclosure provides a method of preparing Compound A, the method including:
a) coupling Compound 6a and Compound 9 to form Compound 12:

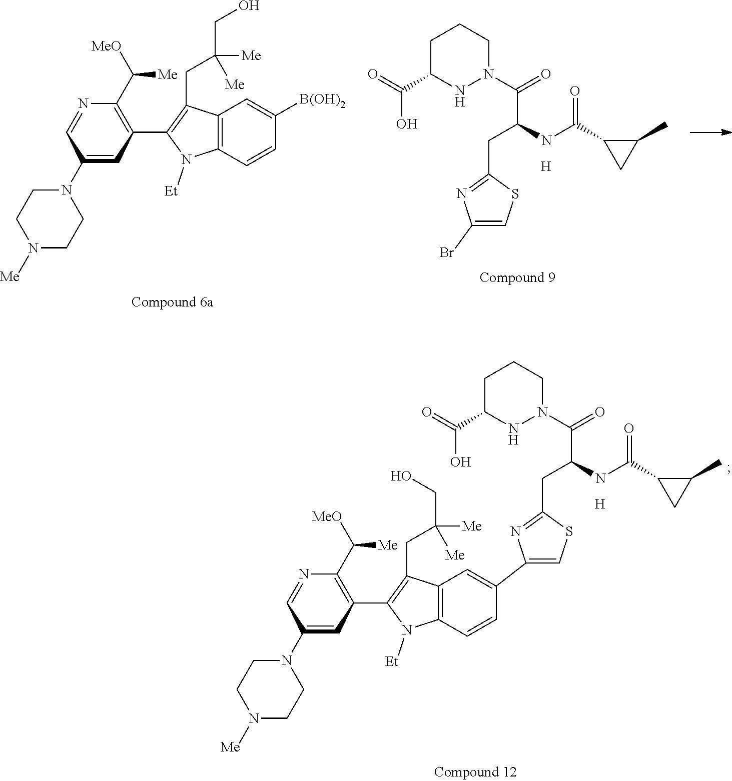

and
b) lactonizing Compound 12 to form compound A:

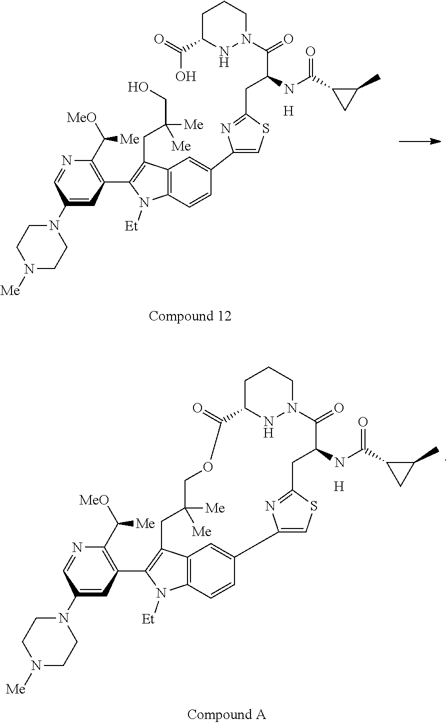

In some embodiments, the coupling step (a) includes contacting Compound 6a and Compound 9 with a palladium catalyst. In some embodiments, the palladium catalyst is Pd(dtbpf)Cl₂. In some embodiments, the coupling step (a) further includes contacting Compound 6a and Compound 12 with a base (e.g., potassium carbonate). In some embodiments, the coupling step (a) is carried out above room temperature (e.g., above 20, above 30, above 40, above 50, above 60, or above 70° C.). In some embodiments, the coupling step (a) is carried out between 7° and 80° C.

In some embodiments, the coupling step (a) is carried out according to the following scheme:

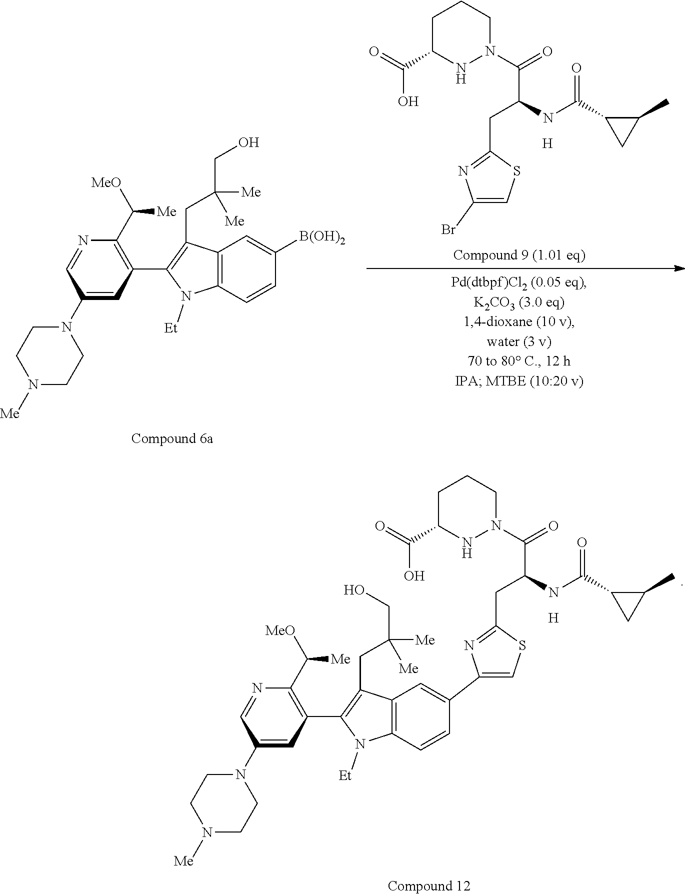

In some embodiments, the lactonizing step (b) includes contacting Compound 12 with EDCI. In some embodiments, the lactonizing step (b) further includes contacting Compound 12 with one or more bases (e.g., DMAP and/or DIPEA). In some embodiments, the lactonizing step (b) further includes contacting Compound 12 with HOBt.

In some embodiments, the lactonizing step (b) is carried out according to the following scheme:

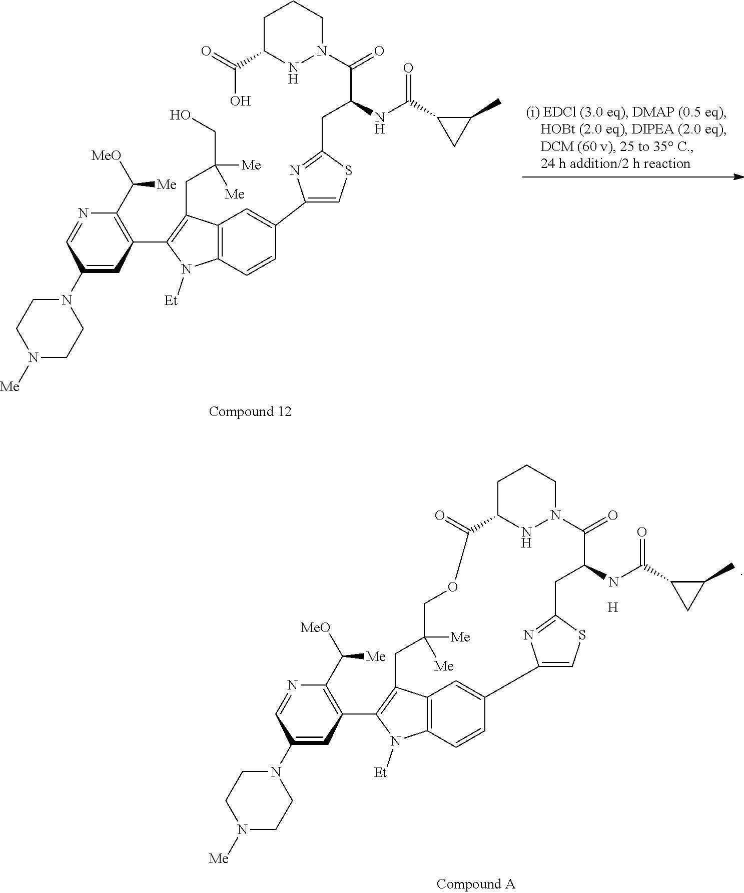

In some embodiments of the methods of preparing Compound A, the method further includes purifying Compound A. In some embodiments, the purifying includes forming a salt of Compound A. In some embodiments, the salt of Compound A is a hydrochloride salt of Compound A. In some embodiments, the salt of Compound A is a lactate salt of Compound A. In some embodiments, the lactate salt of Compound A is formed by contacting Compound A with lactic acid (e.g., with 1 equivalent of lactic acid, with 2 equivalents of lactic acid, with 3 equivalents of lactic acid, or with 4 equivalents of lactic acid). In some embodiments, the contacting Compound A with lactic acid is carried out in a solvent (e.g., acetonitrile).

In some embodiments, the purifying includes converting the salt of Compound A to a free base form of Compound A. In some embodiments, converting the salt of Compound A to a free base form of Compound A includes contacting the salt of Compound A with a base. In some embodiments, the base is sodium carbonate. In some embodiments, the contacting of the salt of Compound A with a base is carried out in an organic solvent (e.g., an ethereal solvent such as 2-methyltetrahydrofuran). In some embodiments, the method further includes precipitating the free base form of Compound A from a solution. In some embodiments, the precipitating includes adding heptane to the solution of the free base form of Compound A.

In some embodiments, the purifying includes recrystallizing Compound A. In some embodiments, the recrystallizing includes adding a first solvent followed by adding a second solvent. In some embodiments, the first solvent is a protic solvent. In some embodiments, the first solvent is methanol. In some embodiments, the second solvent is water.

### Compound and Intermediates

The present disclosure provides compounds and intermediates useful in the preparation of Compound A. For example, in some embodiments, the disclosure provides a compound having the structure of Compound 2:

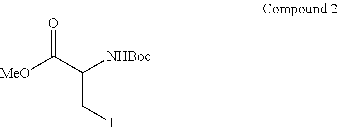 [SMILES: COC(=O)C(CI)NC(=O)OC(C)(C)C]

or a salt thereof.

In some embodiments, the compound has the structure of Compound 2a:

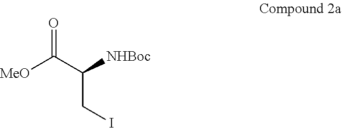 [SMILES: COC(=O)[C@H](CI)NC(=O)OC(C)(C)C]

or a salt thereof.

In another aspect, the disclosure provides a compound having the structure of Compound 4:

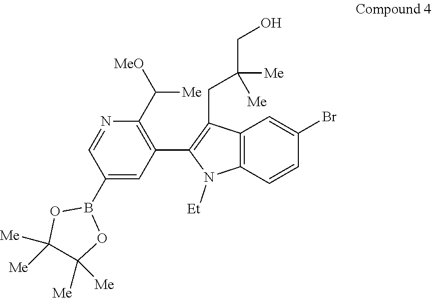 [SMILES: CCn1c(-c2cc(B3OC(C)(C)C(C)(C)O3)cnc2C(C)OC)c(CC(C)(C)CO)c2cc(Br)ccc21]

or a salt thereof.

In some embodiments, the compound has the structure of Compound 4a:

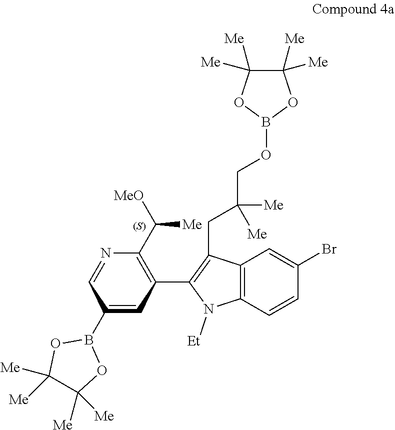

or a salt thereof.

In yet another aspect, the disclosure provides a compound having the structure of Compound 5:

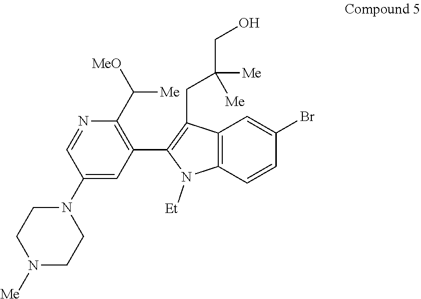 [SMILES: CCn1c(-c2cc(N3CCN(C)CC3)cnc2C(C)OC)c(CC(C)(C)CO)c2cc(Br)ccc21]

or a salt thereof.

In some embodiments, the compound has the structure of Compound 5a:

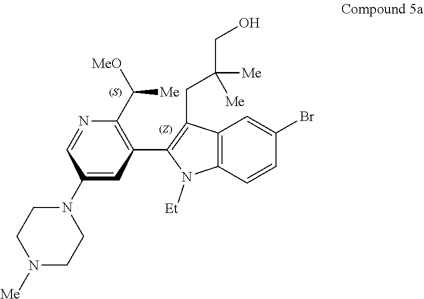

or a salt thereof.

In still another aspect, the disclosure provides a compound having the structure of Compound 6:

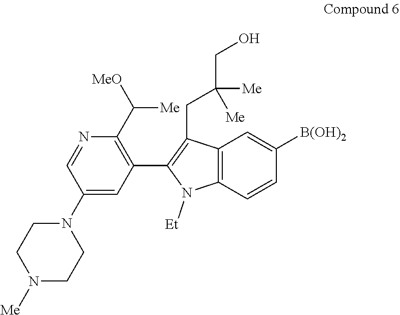 [SMILES: CCn1c(-c2cc(N3CCN(C)CC3)cnc2C(C)OC)c(CC(C)(C)CO)c2cc(B(O)O)ccc21]

or a salt thereof.

In some embodiments, the compound has the structure of Compound 6a:

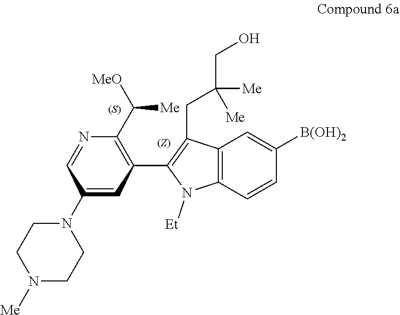

or a salt thereof.

In another aspect, the disclosure provides a compound having the structure of Formula I:

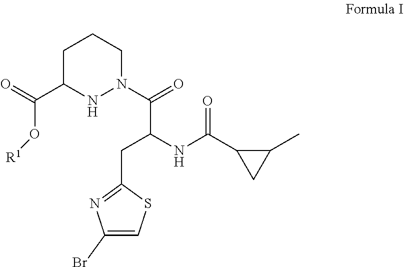

or a salt thereof, wherein R¹ is H or C₁-C₆ alkyl.

In some embodiments, the compound has the structure of Formula Ia:

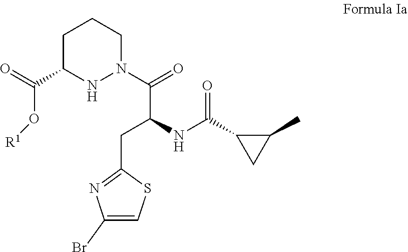

or a salt thereof.

In some embodiments, R¹ is H. In some embodiments, R¹ is CH₃.

In some embodiments, the disclosure provides a compound having the structure of Compound 9c:

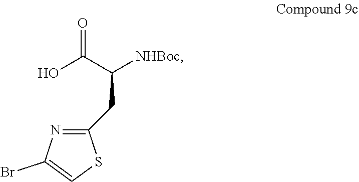 [SMILES: CC(C)(C)OC(=O)N[C@@H](Cc1nc(Br)cs1)C(=O)O]

or a salt thereof.

In some embodiments, the disclosure provides a compound having the structure of Compound 10:

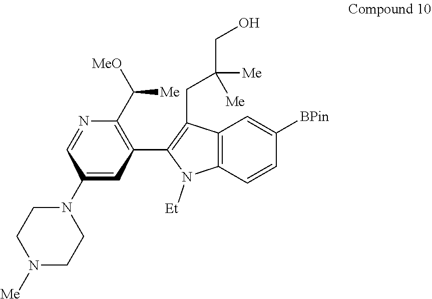

or a salt thereof.

In some embodiments, the disclosure provides a compound having the structure of Compound 11:

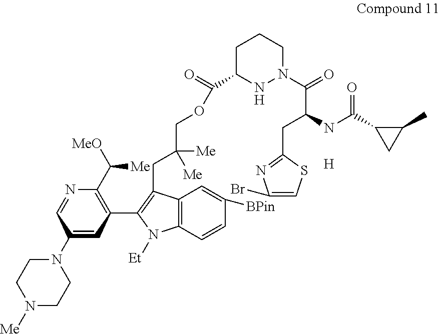

or a salt thereof. In some embodiments, the Br can be replaced by a halogen that is suitable for Suzuki reactions (e.g., iodide or chloride). In some embodiments, the BPin can be replaced by a boronic ester that is suitable for Suzuki reactions (e.g., neopentyl- and catechol boronic esters).

In some embodiments, the disclosure provides a compound having the structure of Compound 12:

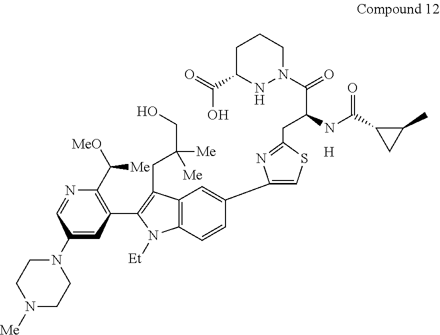

or a salt thereof.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Example 1. Synthetic Procedure for Compound 13b—(S)-3-Bromo-2-(1-Methoxyethyl)Pyridine

Detailed below is a general synthetic procedure for Compound 13b—(S)-3-bromo-2-(1-methoxyethyl)pyridine.

Synthesis of Compound 13b—(S)-3-Bromo-2-(1-Methoxyethyl)Pyridine.

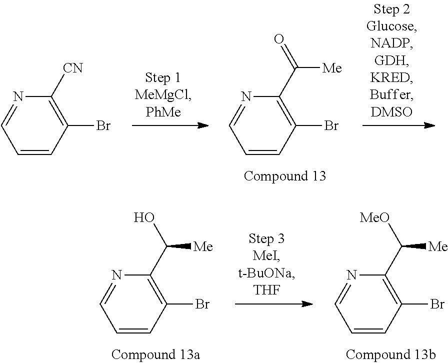 [SMILES: N#Cc1ncccc1Br ; CC(=O)c1ncccc1Br ; C[C@H](O)c1ncccc1Br ; CO[C@@H](C)c1ncccc1Br]

### Part 1—Synthesis of Compound 13-1—3-Bromopyridin-2-yl)ethan-1-One.

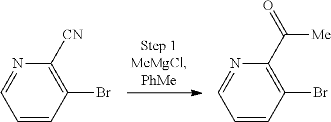 [SMILES: N#Cc1ncccc1Br ; CC(=O)c1ncccc1Br]

To a reactor was charged toluene (2,100 L, 7 V) and 3-bromopicolinonitrile (300 kg, 1,639 mol, 1 equiv). The resulting mixture was cooled to, and maintained at, −20° C. To this was charged MeMgCl (3 M in THF, 601 L, 1,803 mol, 1.1 equiv). The resulting mixture was heated to, and maintained at, 10-20° C. for 16 hours at which point HPLC analysis showed reaction completion.

The reaction mixture was charged into pre-cooled (−10 to 0° C.) 4 M aqueous HCl (1,070 L, 2.6 equiv) at −10 to 10° C. and resulting mixture was maintained at 15-25° C. for 30 minutes. The phases were separated, and the aqueous phase extracted with toluene (600 L×5, 2 V×5). The combined organic layers were washed with saturated aqueous NaHCO₃ (100 L, 0.3 V) and then concentrated (50-60° C., −0.08 MPa) to ˜200 L (0.7 V) to afford crude 1-(3-bromopyridin-2-yl)ethan-1-one (Compound 13) (346 kg, 93.7% a/a purity, 83.4% w/w assay, 88% yield, Table 1) as a brown oil which was used directly in the next step.

TABLE 1
HPLC Method for Part 1 of Example 1

| Column: | XSelect CSH Phenyl-Hexyl C18 | | |
|---|---|---|---|
| | (4.6 × 150 mm, 3.5 μm) | | |
| Mobile Phase: | A: 10 mM HCO₂NH₄ in water (pH 3.7) | | |
| | B: MeCN | | |
| Gradient: | Time (min) | A % | B % |
| | 0.0 | 80 | 20 |
| | 9.0 | 50 | 50 |
| | 11.0 | 5 | 95 |
| | 14.0 | 5 | 95 |

| | |
|---|---|
| Flow Rate: | 1.0 mL/min |
| UV Detector | 210 nm |
| Wavelength: | |
| Column Temperature: | 15° C. |
| Retention Times: | 3-bromopicolinonitrile: 8.5 min |
| | 1-(3-bromopyridin-2-yl)ethan-1-one: 8.1 min |

LRMS (ESI+)

Calculated for C₇H₇BrNO (M+H⁺): 199.97110

Found: 200.0

¹H NMR (400 MHz, DMSO-d₆, 25° C.)

δ 8.66 (dd, J=4.6, 1.3 Hz, 1H), 8.22 (dd, J=8.2, 1.3 Hz, 1H), 7.52 (dd, J=8.2, 4.6 Hz, 1H), 2.61 (s, 3H).

Part 2—Synthesis of Compound 13a—(S)-1-(3-bromopyridin-2-yl)ethan-1-oL.

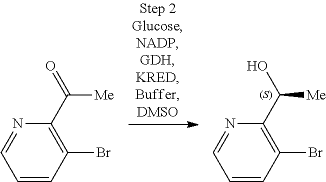

To a reactor was charged potassium phosphate buffer (0.2 M, pH=6-8-7.2, 2,000 L, 10 V), glucose (594 kg, 3,297 mol, 3.3 equiv), GDH (4 kg, 2% w/w), NADP (2 kg, 1% w/w), KRED (2 kg, 1% w/w), and a solution of 1-(3-bromopyridin-2-yl)ethan-1-one (Compound 13) (200 kg, 999.83 mol, 1 equiv) in DMSO (200 L, 1 V) at 25-30° C. Note: the pH was maintained at 6-5-7 as needed, using 2 M aqueous NaOH. The reaction mixture was maintained at 28-32° C. for 6 hours at which point HPLC analysis showed reaction completion.

To the reaction mixture was charged diatomaceous earth (40 kg, 20% w/w) and MTBE (800 L, 4 V). The resulting mixture was filtered, and the cake was washed with MTBE (200 L, 1 V). The resulting phases were separated, and the aqueous phase extracted again with MTBE (500 L×3, 2.5 V×3). The combined organic phases were washed with brine (100 L, 0.5 V) and concentrated (45-55° C., −0.08 MPa) to afford (S)-1-(3-bromopyridin-2-yl)ethan-1-ol (Compound 13a) (204 kg, 97.8% a/a purity, 89.6% w/w assay, 90% yield).

Part 3—Alternative Synthesis of Compound 13a—(S)-1-(3-bromopyridin-2-yl)ethan-1-ol.

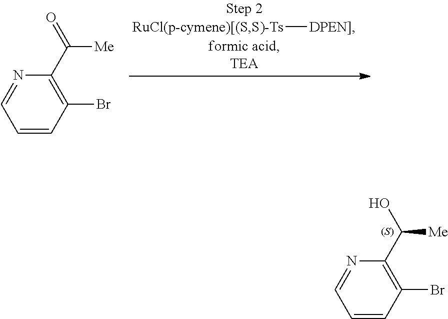

To a reactor was charged triethylamine (47 kg, 464.46 mol, 2.8 equiv). This was cooled to, and maintained at, 0-10° C. To this was charged formic acid (19 kg, 412.82 mol, 2.5 equiv) and RuCl(p-cymene)[(S,S)-Ts-DPEN] (0.55 kg, 864.49 mmol, 0.005 equiv). The resulting mixture was heated to, and maintained at, 30-35° C. To this was charged 1-(3-bromopyridin-2-yl)ethan-1-one (Compound 13) (36.7 kg, 165.12 mol, 1 equiv), rinsing the charging port with additional triethylamine (2 kg, 19.76 mol, 0.12 equiv). The reaction mixture temperature was maintained at 30-35° C. for 6 hours at which point HPLC analysis showed reaction completion.

The reaction mixture was concentrated (30-35° C.) to remove triethylamine. To the resulting mixture was charged water (170 kg) and EtOAc (310 kg) at 15-25° C. The phases were separated, and the aqueous phases extracted with EtOAc (160 kg×2). The combined organic phases were washed with brine (158 kg×2), dried over anhydrous Na₂SO₄, and filtered, washing the spent drying agent cake with EtOAc (40 kg). The combined filtrates were cooled to, and maintained at, 0-10° C. and to this was charged 35% w/w HCl in MeOH (55 kg, 3.2 equiv). The resulting mixture was maintained at 0-10° C. for 12 hours before being filtered, washing the product with EtOAc (40 kg). The product was dissolved in water (66 kg) and EtOAc (170 kg) and the resulting solution was cooled to, and maintained at, 5-15° C. To this was charged a solution of NaHCO₃ (33 kg) in water (170 kg). The phases were separated, and the aqueous phases extracted with EtOAc (170 kg×3). The combined organic phases were washed with brine (158 kg×2), dried over anhydrous Na₂SO₄, and filtered, washing the spent drying agent cake with EtOAc (120 kg). The filtrate was concentrated (40-45° C.) to afford (S)-1-(3-bromopyridin-2-yl)ethan-1-ol (Compound 13a) (30.0 kg, >99.9% a/a purity, 95% w/w assay, 86% yield, Table 2) as a dark brown oil.

TABLE 2
HPLC Method for Part 2 and 3 of Example 1

| Column: | XSelect CSH Phenyl-Hexyl C18 | | |
|---|---|---|---|
| | (4.6 × 150 mm, 3.5 μm) | | |
| Mobile Phase: | A: 10 mM HCO₂NH₄ in water (pH 3.7) | | |
| | B: MeCN | | |
| Gradient: | Time (min) | A % | B % |
| | 0.0 | 80 | 20 |
| | 9.0 | 50 | 50 |
| | 11.0 | 5 | 95 |
| | 14.0 | 5 | 95 |

| | |
|---|---|
| Flow Rate: | 1.0 mL/min |
| UV Detector | 210 nm |
| Wavelength: | |
| Column Temperature: | 15° C. |
| Retention Times: | 1-(3-bromopyridin-2-yl)ethan-1-one: 8.0 min |
| | (S)-1-(3-bromopyridin-2-yl)ethan-1-ol: 5.6 min |

LRMS (ESI+)

Calculated for C₇H₉BrNO (M+H⁺): 201.98675

Found: 202.0

¹H NMR (400 MHz, DMSO-d₆, 25° C.)

δ 8.56 (dd, J=4.6, 1.4 Hz, 1H), 8.02 (dd, J=8.0, 1.4 Hz, 1H), 7.26 (dd, J=8.0, 4.6 Hz, 1H), 5.13-5.04 (m, 2H), 1.37 (d, J=6.0 Hz, 3H).

Part 4—Synthesis of Compound 13b—(S)-3-bromo-2-(1-methoxyethyl)pyridine.

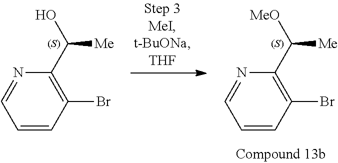

To a reactor was charged THF (2,025 L, 5 V) and t-BuONa (231 kg, 2,404 mol, 1.2 equiv). The resulting mixture was cooled to, and maintained at, 0-10° C. To this was charged a solution of (S)-1-(3-bromopyridin-2-yl)ethan-1-ol (405 kg, 2,004 mol, 1 equiv) in THF (800 L, 2 V) and Mel (340 kg, 2,395 mol, 1.2 equiv). The resulting reaction mixture was maintained at 0-10° C. for 16 hours at which point HPLC analysis showed reaction completion.

To the reaction mixture was charged 7.5% w/w aqueous NH₃ (520 L, 1.3 V) at 0-10° C. and MTBE (1,200 L, 3 V). The phases were separated, and the aqueous layer extracted with MTBE (1,200 L, 3 V). The combined organic phases were washed with brine (200 L, 0.5 V) and concentrated (50-60° C., −0.08 MPa) to afford crude (S)-3-bromo-2-(1-methoxyethyl)pyridine (Compound 13b). The crude (S)-3-bromo-2-(1-methoxyethyl)pyridine was distilled (120° C., 600 Pa) to afford (S)-3-bromo-2-(1-methoxyethyl)pyridine (445 kg, 99.3% a/a purity, 90.2% w/w assay, 93% yield, Table 3) as a colorless solid (solidified after packaging).

TABLE 3
HPLC Method for Part 4 of Example 1

| Column: | XSelect CSH Phenyl-Hexyl C18 | | |
|---|---|---|---|
| | (4.6 × 150 mm, 3.5 μm) | | |
| Mobile Phase: | A: 10 mM HCO₂NH₄ in water (pH 3.7) | | |
| | B: MeCN | | |
| Gradient: | Time (min) | A % | B % |
| | 0.0 | 80 | 20 |
| | 9.0 | 50 | 50 |
| | 11.0 | 5 | 95 |
| | 14.0 | 5 | 95 |

| | |
|---|---|
| Flow Rate: | 1.0 mL/min |
| UV Detector | 210 nm |
| Wavelength: | |
| Column Temperature: | 15° C. |
| Retention Times: | (S)-1-(3-bromopyridin-2-yl)ethan-1-ol: 5.6 min |
| | (S)-3-bromo-2-(1-methoxyethyl)pyridine: 7.8 min |

LRMS (ESI+)

Calculated for C₈H₁₁BrNO (M+H⁺): 216.00240

Found: 216.00

¹H NMR (400 MHz, CDCl₃, 25° C.)

δ 8.61 (d, J=3.2 Hz, 1H), 7.83 (q, J=1.6, 6.8 Hz, 1H), 7.08 (q, J=3.6, 4.8 Hz, 1H), 4.92 (q, J=6.4 Hz, 1H), 3.31 (s, 3H), 1.48 (d, J=6.8 Hz, 3H).

### Example 2. Synthetic Procedure for 3,3-dimethyldihydro-2H-pyran-2,6(3H)-dione

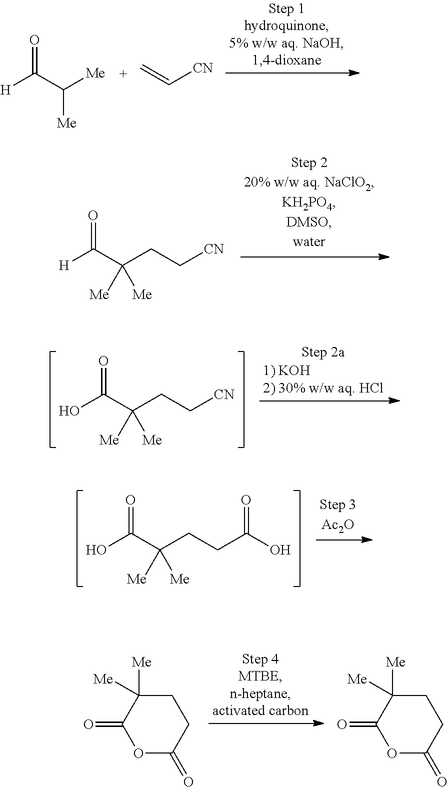

Part 1—Synthesis of 4,4-dimethyl-5-oxopentanenitrile.

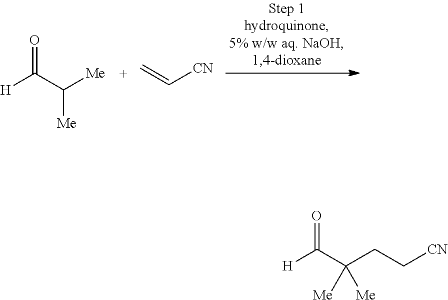

To a reactor was charged 1,4-dioxane (1,552 L, 5 V), hydroquinone (1.55 kg, 14.1 mol, 0.0033 equiv), and 5% w/w aqueous NaOH (341.4 kg, 426.78 mol, 0.1 equiv). The resulting mixture was heated to, and maintained at, 70-75° C. To this was charged isobutyraldehyde (310.6 kg, 4,307.3 mol, 1 equiv) and acrylonitrile (2) (285.7 kg, 5,384.5 mol, 1.25 equiv) over 8 hours. The reaction mixture was maintained at 70-7500 for 8 hours at which point GO analysis showed reaction completion.

The reaction mixture was then cooled to, and maintained at, 20-25° C. The pH was adjusted to 5-6 with 3.5% w/w aqueous HCl (required 172.5 kg) and concentrated (45° C., ˜0.03 atm) until no organic solvent was distilled. The remaining residue was cooled to, and maintained at, 20-25° C. To this was charged DCM (1,552 L, 5 V) and water (620 L, 2 V). The phases were separated, and the organic phase concentrated (45° C., ˜0.03 atm) until no solvent was distilled to afford crude 4,4-dimethyl-5-oxopentanenitrile as a brown oil (626.6 kg, 70.8% a/a purity, 43.5% w/w assay, 51% yield, Table 4).

TABLE 4
GC Method for Part 1 of Example 2

| Column | HP-5: 30 m*0.32 mm*0.25 μm | | | |
|---|---|---|---|---|
| Detector | Temperature | 260° C. | | |
| parameters | Flow H2 | 40 mL/min | | |
| | Air flow | 300 mL/min | | |
| | Makeup (N2) | 25 mL/min | | |
| Injector | Temperature | 280° C. | | |
| parameters | Split ratio | 30:1 | | |
| Carrier gas | N2 | | | |
| | Flow | 2.5001 mL/min | | |
| | Mode | Constant Flow | | |
| Oven | Time | Rate | Temperature | Hold |
| parameters | [min] | [° C./min] | [° C.] | [min] |
| | 0.0 | — | 40 | 3 |
| | | 20 | 260 | 3 |

| | |
|---|---|
| Retention time | isobutyraldehyde: 1.447 min |
| | 4,4-dimethyl-5-oxopentanenitrile: 7.043 min |

LRMS (ESI+)

Calculated for C₇H₁₂NO (M+H⁺): 126.09189

Found: 126.0

¹H NMR (400 MHz, CDCl₃, 25° C.)

δ 9.37 (s, 1H), 2.30-2.19 (m, 2H), 1.88-1.77 (m, 2H), 1.06 (s, 6H).

Part 2—Synthesis of Crude 3,3-dimethyldihydro-2H-pyran-2,6(3H)-dione.

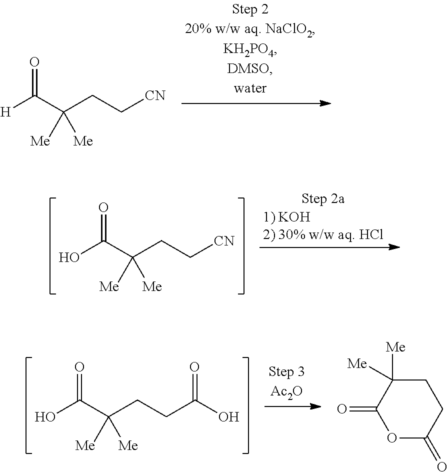

To a reactor was charged water (1,115 kg, 5 V), KH₂PO₄ (13.8 kg, 101.4 mol, 0.057 equiv), DMSO (164.0 kg, 2,099.1 mol. 1.2 equiv), and crude 4,4-dimethyl-5-oxopentanenitrile (455.3 kg, 49.0% w/w assay, 1,782.3 mol, 1 equiv). The resulting mixture was cooled to, and maintained at, 10-20° C. To this was charged 20% w/w aqueous NaClO₂ (1,185.0 kg, 2,620.5 mol, 1.5 equiv) over 20 hours. The reaction mixture was then maintained at a temperature of 10-20° C. for 1 hour at which point GC analysis showed reaction completion. This afforded crude 4-cyano-2,2-dimethylbutanoic acid which was used directly in the next step.

To the mixture of crude 4-cyano-2,2-dimethylbutanoic acid was charged KOH (361.5 kg, 6,442.7 mol, 3.6 equiv). The resulting mixture was extracted with MTBE (800 kg×2, 4.9 V×2). The aqueous phase was then heated to, and maintained at, 90-100° C. for 15 hours at which point GC analysis showed reaction completion.

The reaction mixture was cooled to, and maintained at, 15-25° C. The pH was adjusted to 1-2 with 30% w/w aqueous HCl (required 1,058 kg, 4.9 equiv). The resulting mixture was extracted with MTBE (1,058 kg×2, 6.4 V×2). The combined organic phases were washed with 5% w/w aqueous NaCl (378 kg×2, 1.7 V×2) and concentrated (40-45° C., ˜0.03 atm) to 670 L (3 V) affording crude 2,2-dimethylpentanedioic acid which was used directly in the next step.

To the mixture of crude 2,2-dimethylpentanedioic acid was charged Ac₂O (614.6 kg, 6,020.1 mol, 3.4 equiv) at 40-45° C. The resulting mixture was concentrated (40-45° C., ˜0.03 atm) to remove MTBE. The reaction mixture was heated to, and maintained at, 80-85° C. for 2 hours at which point GC analysis showed reaction completion.

The reaction mixture was then concentrated (70-75° C., ˜0.03 atm) to remove AcOH and Ac2O until no solvent was distilled. This afforded crude 3,3-dimethyldihydro-2H-pyran-2,6(3H)-dione (390.5 kg, 86.3% a/a purity, 69.3% w/w assay, 107% crude yield) which was used directly in the next step.

Part 3—Synthesis of 3,3-dimethyldihydro-2H-pyran-2,6(3H)-dione.

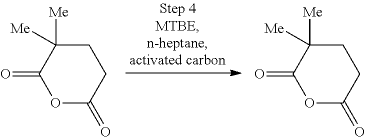 [SMILES: CC1(C)CCC(=O)OC1=O ; CC1(C)CCC(=O)OC1=O]

To a reactor was charged n-heptane (574.0 kg, 1.86 V for crude weight). This was cooled to, and maintained at, −10 to −5° C. To this was charged a solution of crude 3,3-dimethyldihydro-2H-pyran-2,6(3H)-dione (454.0 kg, 84.1% w/w assay) in MTBE (667.2, 2 V for crude weight) over 10 hours. The resulting mixture was maintained at −10 to −5° C. for 1.5 hours and then filtered.

The filter cake was dissolved in MTBE (572 kg, 2 V for assay weight). To the resulting solution was charged activated carbon (19.1 kg, 0.05% w/w for assay weight). This was maintained at 15-25° C. for 8 hours. The resulting solution was then filtered, washing the spent carbon cake with MTBE (18 kg, 0.05 V). The filtrate was then added to pre-cooled (−10 to −5° C.) n-heptane (518.4 kg, 2 V for assay weight) over 9 hours. The resulting mixture was maintained at −10 to −5° C. for 2 hours. This was then filtered at −10 to −5° C. The product was dried (25-30° C., ˜0.03 atm) for 16 hours to afford 3,3-dimethyldihydro-2H-pyran-2,6(3H)-dione (212.0 kg, 100% a/a purity, 98.3% w/w assay, 55% yield) as an off-white solid.

Part 4—Alternative Synthesis of 3,3-dimethyldihydro-2H-pyran-2,6(3H)-dione.

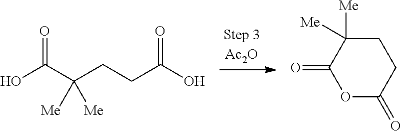 [SMILES: CC(C)(CCC(=O)O)C(=O)O ; CC1(C)CCC(=O)OC1=O]

To a reactor was charged Ac₂O (5.4 L) and 2,2-dimethylpentanedioic acid (2,573 g, 98.8% w/w assay and 506 g, 90.9% w/w assay, 18.74 mol, 1 equiv). The resulting reaction mixture was heated to, and maintained at, 110° C. for 1 hour at which point GC analysis showed reaction completion.

The reaction mixture was concentrated (70° C., ˜0.03 atm) to remove AcOH and Ac₂O until no solvent was distilled. The residue was combined with another batch (2,2-dimethylpentanedioic acid (6,610 g, 90.8% w/w assay)) and distilled (110-120° C., ˜0.005 atm) until no product was distilled. The obtained fraction was triturated with n-heptane (35 L), filtered, and the product dried (25° C., ˜0.005 atm) to afford 3,3-dimethyldihydro-2H-pyran-2,6(3H)-dione (6.66 kg, 99.6% a/a purity, 98.56% w/w assay, 82% yield, Table 5) as an off-white solid.

TABLE 5
GC Method for Part 2, 3, and 4 of Example 2

| Column | HP-5: 30 m*0.32 mm*0.25 μm | | | |
|---|---|---|---|---|
| Detector | Temperature | 260° C. | | |
| parameters | Flow H2 | 40 mL/min | | |
| | Air flow | 300 mL/min | | |
| | Makeup (N2) | 25 mL/min | | |
| Injector | Temperature | 280° C. | | |
| parameters | Split ratio | 30:1 | | |
| Carrier gas | N2 | | | |
| | Flow | 2.5 mL/min | | |
| | Mode | Constant Flow | | |
| Oven | Time | Rate | Temperature | Hold |
| parameters | [min] | [° C./min] | [° C.] | [min] |
| | 0.0 | — | 40 | 3 |
| | | 20 | 260 | 3 |

| | |
|---|---|
| Retention time | 4,4-dimethyl-5-oxopentanenitrile: 6.931 min |
| | 4-cyano-2,2-dimethylbutanoic acid: 8.971 min |
| | 2,2-dimethylpentanedioic acid: 9.952 min |
| | 3,3-dimethyldihydro-2H-pyran-2,6(3H)-dione: 8.073 min |

LRMS (ESI+)

Calculated for C₇H₁₁O₃ (M+H⁺): 143.07082

Found: 143.0

¹H NMR (300 MHz, CDCl₃, 25° C.)

δ 2.82 (t, J=7.0 Hz, 2H), 1.85 (t, J=7.0 Hz, 2H), 1.35 (s, 6H).

Part 5—Alternative Synthesis of 2,2-dimethylpentanedioic Acid.

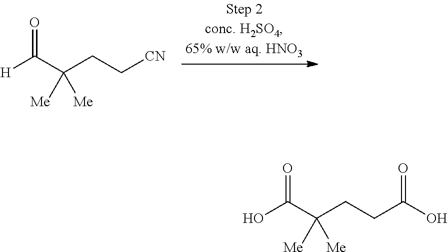 [SMILES: CC(C)(C=O)CCC#N ; CC(C)(CCC(=O)O)C(=O)O]

To a reactor was charged 65% w/w aqueous HNO₃ (3.3 L) and concentrated H₂SO₄ (500 mL) at 25° C. The resulting mixture was heated to, and maintained at, 70-80° C. To this was charged 4,4-dimethyl-5-oxopentanenitrile (2.21 kg, 90.5% w/w assay, 15.98 mol, 1 equiv) in portions over 24 hours. The reaction mixture was then maintained at 70-75° C. for 1 hour at which point GC analysis showed reaction completion.

The reaction mixture was cooled to 25° C. and then charged into ice cold water (10 kg) during which time solid precipitated. The resulting mixture was extracted with MTBE (10 L×1 followed by 5 L×2). The combined organic phases were washed with water (2 L×1), washed with brine (2 L×1), dried over anhydrous Na₂SO₄, filtered, and then concentrated (45° C., ˜0.03 atm) until no solvent was distilled. This afforded 2,2-dimethylpentanedioic acid (2.6 kg, 98.8% w/w assay, 100% yield, Table 6) as a white solid.

TABLE 6
GC Method for Part 5 of Example 2

| Column | HP-5: 30 m*0.32 mm*0.25 μm | | | |
|---|---|---|---|---|
| Dector | Temperature | 260° C. | | |
| parameters | Flow H2 | 40 mL/min | | |
| | Air flow | 300 mL/min | | |
| | Makeup (N2) | 25 mL/min | | |
| Injector | Temperature | 280° C. | | |
| parameters | Split ratio | 30:1 | | |
| Carrier gas | N2 | | | |
| | Flow | 2.5 mL/min | | |
| | Mode | Constant Flow | | |
| Oven | Time | Rate | Temperature | Hold |
| parameters | [min] | [° C./min] | [° C.] | [min] |
| | 0.0 | — | 40 | 3 |
| | | 20 | 260 | 3 |

| | |
|---|---|
| Retention time | 4,4-dimethyl-5-oxopentanenitrile: 6.931 min |
| | 2,2-dimethylpentanedioic acid: 9.952 min |

LRMS (ESI−)

Calculated for C₇H₁₁O₄ (M−H⁺): 159.06573

Found: 159.2

¹H NMR (400 MHz, CDCl₃, 25° C.)

δ 2.42 (t, J=7.5 Hz, 2H), 1.92 (t, J=7.5 Hz, 2H), 1.23 (s, 6H).

### Example 3. Synthetic Procedure for Compound 2a—methyl(R)-2-((tert-butoxycarbonyl)amino)-3-iodopropanoate

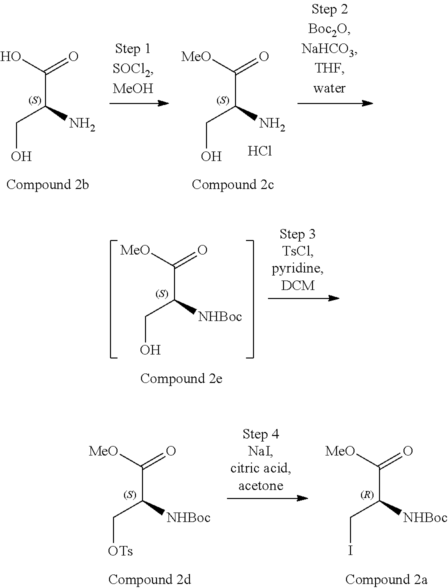

Part 1—Synthesis of Compound 2c—methyl L-serinate hydrochloride.

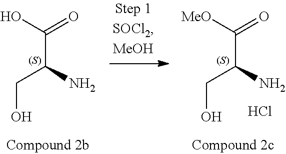

To a reactor was charged MeOH (1,650 L, 3 V) and L-serine (Compound 2b) (550 kg, 5,233 mol, 1 equiv). The resulting mixture was cooled to, and maintained at, 0-10° C. To this was charged SOCl₂ (695 kg, 5,842 mol, 1.1 equiv) over 12 hours. The resulting reaction mixture was warmed to, and maintained at, 20-30° C. for 5 hours at which point HPLC monitoring showed reaction completion.

The reaction mixture was concentrated (40-45° C., −0.09 MPa) to 1.5 V. To the resulting residue was charged MTBE (1,650 L, 3 V) and the resulting mixture was concentrated (40-45° C., −0.09 MPa) to 1.5 V. To the resulting residue was charged MTBE (1,650 L, 3 V) and the resulting mixture was cooled to, and maintained at, 5-15° C. for 1 hour. This was then filtered at 5-15° C., washing the cake with MTBE (203.5 kg, 0.5 V). The cake was dried (40-50° C., −0.09 MPa) to afford methyl L-serinate hydrochloride (Compound 2c) (814.0 kg, 99.7% a/a purity, 100% yield, Table 7).

TABLE 7
HPLC Method for Part 1 of Example 3

| Column | Waters Atlantis T3 4.6 mm × 150 mm, 3 μm | | |
|---|---|---|---|
| Buffer A | 0.1% TFA-0.2% Heptafluorobutyricacid in water | | |
| Buffer B | Methanol | | |
| Column Temperature | 35° C. | | |
| Flow rate | 1.0 mL/min | | |
| Detector | ELSD | | |
| Evaporator | 45° C. | | |
| Temperature | | | |
| Nebulizer Temperature | 40° C. | | |
| Gas flow rate | 1.6SLM | | |
| LED Intensity | 100% | | |
| Data Rate | 80 Hz | | |
| Injection Volume | 5 μL | | |
| Reference solution | 4.0 mg/mL | | |
| concentration | | | |
| Sample concentration | 400.0 mg/mL | | |
| Diluent | Water | | |
| Run Time | 20.0 min | | |
| Gradient | Time (min) | A (%) | B (%) |
| | 0.0 | 100 | 0 |
| | 4.0 | 100 | 0 |
| | 8.0 | 50 | 50 |
| | 10.0 | 20 | 80 |
| | 15.0 | 20 | 80 |
| | 15.1 | 100 | 0 |
| | 20.0 | 100 | 0 |

| | |
|---|---|
| Retention Time | L-serine: 3.2 min |
| | methyl L-serinate hydrochloride: 8.2 min |

LRMS (ESI+)

Calculated for C₄H₁₁NO₃ (M+H⁺): 120.06607

Found: 120.1

¹H NMR (400 MHz, CD₃OD, 25° C.)

δ 4.16-4.10 (m, 1H), 4.03-3.88 (m, 2H), 3.83 (s, 3H).

Part 2—Synthesis of Compound 2e—methyl (tert-butoxycarbonyl)-L-serinate.

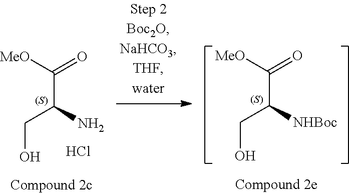

To a reactor was charged water (813 kg, 2 V) and methyl L-serinate hydrochloride (Compound 2c) (407 kg, 2,616 mol, 1 equiv). The resulting mixture was cooled to, and maintained at, 10-20° C.

To a separate reactor was charged THE (723 kg, 2 V) and NaHCO₃ (659 kg, 7,844 mol, 3 equiv). The resulting mixture was cooled to, and maintained at, 10-20° C. To this was charged the solution of methyl L-serinate hydrochloride (Compound 2c) over 1 hour. To the resulting mixture was charged Boc₂O (627 kg, 2,873 mol, 1.1 equiv) over 2.5 hours. The resulting reaction mixture was warmed to, and maintained at, 20-30° C. for 1 hour at which point HPLC monitoring showed reaction completion.

The reaction mixture was filtered, washing the cake with DCM (541.3 kg×2, 1 V×2). The filtrate was then concentrated (45-55° C.) to 1.5 V. To the resulting residue was charged DCM (2,706 kg, 5 V). The phases were separated and the aqueous phase extracted with DCM (2,706 kg, 5 V). The combined organic phases were washed with water (1,221 kg×3, 3 V×3), washed with brine (1,628 kg, 3 V), dried over anhydrous Na₂SO₄, and filtered, washing the cake with DCM (272.7 kg, 0.5 V). The resulting filtrate was concentrated (s 40° C.) to 5 V, affording crude methyl (tert-butoxycarbonyl)-L-serinate (Compound 2e, Table 8) which was used directly in the next step.

TABLE 8
HPLC Method for Part 2 of Example 3

| Column | Waters Atlantis T3 4.6 mm × 150 mm, 3 μm | | |
|---|---|---|---|
| Buffer A | 0.1% TFA-0.2% Heptafluorobutyricacid in water | | |
| Buffer B | Methanol | | |
| Column Temperature | 35° C. | | |
| Flow rate | 1.0 mL/min | | |
| Detector | ELSD | | |
| Evaporator | 45° C. | | |
| Temperature | | | |
| Nebulizer Temperature | 40° C. | | |
| Gas flow rate | 1.6SLM | | |
| LED Intensity | 100% | | |
| Data Rate | 80 Hz | | |
| Injection Volume | 5 μL | | |
| Reference solution | 3.0 mg/mL | | |
| concentration | | | |
| Sample concentration | 300.0 mg/mL | | |
| Diluent | Methanol | | |
| Run Time | 20.0 min | | |
| Gradient | Time (min) | A (%) | B (%) |
| | 0.0 | 100 | 0 |
| | 4.0 | 100 | 0 |
| | 8.0 | 50 | 50 |
| | 10.0 | 20 | 80 |
| | 15.0 | 20 | 80 |
| | 15.1 | 100 | 0 |
| | 20.0 | 100 | 0 |

| | |
|---|---|
| Retention Time | methyl L-serinate hydrochloride: 8.1 min |
| | methyl (tert-butoxycarbonyl)-L-serinate: 12.0 min |

LRMS (ESI+)

Calculated for C₉H₁₇NNaO₅ (M+Na⁺): 242.10044

Found: 242.1

¹H NMR (400 MHz, CDCl₃, 25° C.)

δ 5.56 (d, J=8.1 Hz, 1H), 4.32 (d, J=7.8 Hz, 1H), 3.95-3.78 (m, 2H), 3.73 (s, 3H), 2.83 (s, 1H), 1.40 (s, 9H).

Part 3—Synthesis of Compound 2d—methyl N-(tert-butoxycarbonyl)-O-tosyl-L-serinate.

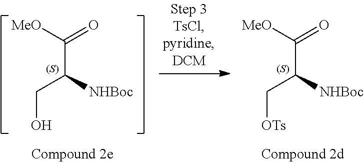

To the DCM solution of crude methyl (tert-butoxycarbonyl)-L-serinate (Compound 2e) (assumed 573.54 kg, 2,616 mol, 1 equiv) was charged TsCl (472.14 kg, 2,477 mol, 0.95 equiv). The resulting mixture was cooled to, and maintained at, −5-5° C. To this was charged pyridine (268.6 kg, 3,396 mol, 1.3 equiv) over 3.5 hours. The reaction mixture was then warmed to, and maintained at, 20-30° C. for 5 hours at which point HPLC monitoring showed reaction completion.

The reaction mixture was diluted with DCM (2,706 kg, 5 V). To this was charged 5% w/w aqueous NaHCO₃ (3 V, 61 kg NaHCO₃, 1,221 kg water) over 0.5 hours. The phases were separated and the organic phase washed with 10% w/w citric acid (3 V, 133 kg citric acid, 1,219 kg water) twice, washed with brine (3 V, 407 kg NaCl, 1,221 kg water), and concentrated (≤40° C.) to 1.5 V. To the resulting residue was charged MTBE (903.5 kg, 3 V) and this was concentrated to 1.5 V. To the resulting residue was charged MTBE (602.3 kg, 2 V) and this was cooled to, and maintained at, −15 to −5° C. To this was then charged n-heptane (1,443 kg, 5 V) over 1 hour and the resulting slurry was maintained at −15 to −5° C. for 6 hours. This was then filtered, washing the cake with pre-cooled (−15 to −5° C.) n-heptane (138 kg×2, 0.5 V×2). The cake was dried (35-4500, −0.09 MPa) for 8 hours to afford methyl N-(tert-butoxycarbonyl)-O-tosyl-L-serinate (Compound 2d) (444 kg, 92.8% a/a purity, 45% yield from methyl L-serinate hydrochloride (Compound 2c), Table 9).

TABLE 9
HPLC Method for Part 3 of Example 3

| Column | Waters Atlantis T3 4.6 mm × 150 mm, 3 μm | | |
|---|---|---|---|
| Buffer A | 0.1% TFA in water | | |
| Buffer B | Methanol | | |
| Column Temperature | 35° C. | | |
| Flow rate | 1.0 mL/min | | |
| Detector | ELSD | | |
| Evaporator Temperature | 45° C. | | |
| Nebulizer Temperature | 40° C. | | |
| Gas flow rate | 1.6SLM | | |
| LED Intensity | 100% | | |
| Data Rate | 80 Hz | | |
| Injection Volume | 5 μL | | |
| Diluent | Methanol | | |
| Run Time | 20.0 min | | |
| Gradient | Time (min) | A (%) | B (%) |
| | 0.0 | 100 | 0 |
| | 2.0 | 100 | 0 |
| | 6.0 | 50 | 50 |
| | 8.0 | 20 | 80 |
| | 15.0 | 20 | 80 |
| | 15.1 | 100 | 0 |
| | 20.0 | 100 | 0 |

| | |
|---|---|
| Retention Time | methyl (tert-butoxycarbonyl)-L-serinate: 10.0 min |
| | methyl N-(tert-butoxycarbonyl)-O-tosyl-L-serinate: 11.4 min |
| | TsCl: 7.8 min |

LRMS (ESI+)

Calculated for C₁₆H₂₃NNaO₇S (M+Na⁺): 396.10929

Found: 396.0

¹H NMR (400 MHz, CDCl₃, 25° C.)

δ 7.81-7.72 (m, 2H), 7.34 (d, J=8.1 Hz, 2H), 5.28 (d, J=8.1 Hz, 1H), 4.53-4.45 (m, 1H), 4.38 (dd, J=10.2, 3.1 Hz, 1H), 4.27 (dd, J=10.1, 3.1 Hz, 1H), 3.69 (s, 3H), 2.44 (s, 3H), 1.41 (s, 9H).

Part 4—Synthesis of Compound 2a—methyl (R)-2-((tert-butoxycarbonyl)amino)-3-iodopropanoate.

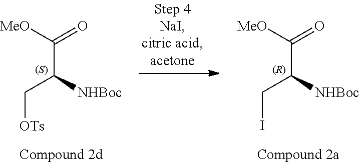

To a reactor was charged NaI (201.6 kg, 1,345 mol, 1.1 equiv), citric acid (119.0 kg, 619 mol, 0.5 equiv), methyl N-(tert-butoxycarbonyl)-O-tosyl-L-serinate (Compound 2d) (458.2 kg, 1,227 mol., 1 equiv), and acetone (2,433 kg, 7 V). The reaction mixture was heated to, and maintained at, 35-45° C. for 20 hours at which point HPLC monitoring showed reaction completion.

The reaction mixture was filtered, washing the cake with EtOAc (824 kg, 2 V). The filtrate was concentrated to 2.5 V and diluted with EtOAc (2,061 kg, 5 V). To this was charged 5% w/w aqueous Na₂S₂O₃ (5 V, 114 kg Na₂S₂O₃, 2,290 kg water). The phases were separated and the aqueous phases extract with EtOAc (1,236 kg, 3 V). The combined organic phases were dried over anhydrous Na₂SO₄ and filtered, washing the cake with EtOAc (207 kg×2, 0.5 V×2). The filtrate was concentrated (35-45° C.) to 1.5 V. To this was charged n-heptane (622 kg, 2 V) and this was concentrated (35-45° C.) to 1.5 V. To this was charged MTBE (33 kg, 0.1 V) and n-heptane (590 kg, 1.9 V). The resulting mixture was warmed to 30° C. and then cooled to, and maintained at, −15 to −5° C. for 6 hours. This was filtered, washing the cake with pre-cooled (−5° C.) n-heptane (155 kg×2, 0.5 V×2). The cake was dried (35° C., −0.09 MPa) for 8 hours to afford crude methyl (R)-2-((tert-butoxycarbonyl)amino)-3-iodopropanoate (Compound 2a).

The crude methyl (R)-2-((tert-butoxycarbonyl)amino)-3-iodopropanoate (Compound 2a) was then dissolved in MeCN (288 kg, 0.8 V) and filtered. The filtrate was extracted with n-heptane (124 kg, x 5, 0.4 V×5). The MeCN phase was then cooled to, and maintained at, −5° C. To this was charged water (1,833 kg, 4 V). The resulting mixture was maintained at −5° C. for 2 hours, filtered, washing the cake with water (916 kg, 2 V). This cake was dissolved in MTBE (33 kg, 0.1 V) and n-heptane (280 kg, 0.9 V) at 30° C. The resulting mixture was then cooled to, and maintained at, −5° C. for 2 hours. This was then filtered, washing the cake with pre-cooled (−5° C.) n-heptane (155 kg×2, 0.5 V×2). The cake was dried (35° C., −0.09 MPa) for 8 hours to afford methyl (R)-2-((tert-butoxycarbonyl)amino)-3-iodopropanoate (Compound 2a) (144.6 kg, 99.7% a/a purity, 36% yield, Table 10) as a white solid.

TABLE 10
HPLC Method for Part 4 of Example 3

| Column | YMC Pack Pro C18 4.6 mm × 150 mm, 5 μm | | |
|---|---|---|---|
| Buffer A | 0.1% TFA in water | | |
| Buffer B | Acetonitrile | | |
| Column Temperature | 25° C. | | |
| Flow rate | 0.8 mL/min | | |
| Detector | DAD | | |
| Wavelength | 252 nm | | |
| Injection Volume | 10 μL | | |
| Sample concentration | 8.0 mg/mL | | |
| Diluent | Acetonitrile | | |
| Run Time | 30.0 min | | |
| Gradient | Time (min) | A (%) | B (%) |
| | 0.0 | 90 | 10 |
| | 3.0 | 45 | 55 |
| | 10.0 | 40 | 60 |
| | 12.0 | 5 | 95 |
| | 25.0 | 5 | 95 |
| | 25.1 | 90 | 10 |
| | 30.0 | 90 | 10 |

| | |
|---|---|
| Retention time | methyl N-(tert-butoxycarbonyl)-O-tosyl-L-serinate: 12.7 min |
| | methyl (R)-2-((tert-butoxycarbonyl)amino)-3- |
| | iodopropanoate: 10.6 min |

HRMS (ESI+)

Calculated for C₄H₉INO₂ (M+H⁺) (des-Boc): 229.96780

Found: 229.9673

¹H NMR (600 MHz, CDCl₃, 25° C.)

δ 5.39 (d, J=6.6 Hz, 1H), 4.53 (t, J=3.6 Hz, 1H), 3.80 (s, 3H), 3.60-3.55 (m, 2H), 1.46 (s, 9H).

### Example 4. Synthetic Procedure for Compound 3—(1S,2S)-2-methylcyclopropane-1-carboxylic acid

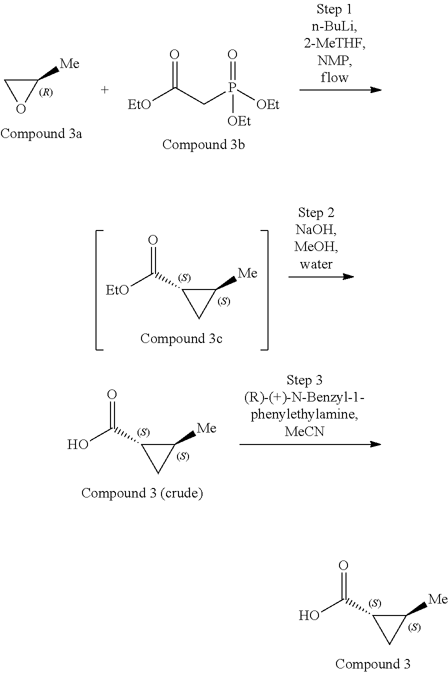

Part 1—Synthesis of Compound 3c—ethyl (1S,2S)-2-methylcyclopropane-1-carboxylate.

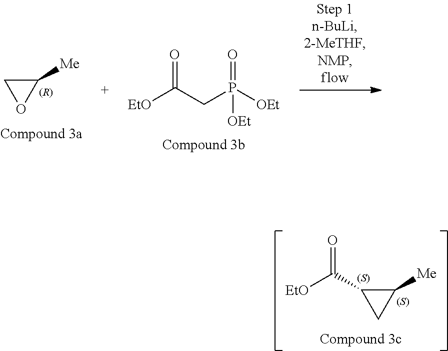

To a reactor was charged n-BuLi (1.6 M in hexanes, 9.79 L, 15.67 mol, 0.91 equiv), ethyl 2-(diethoxyphosphoryl)acetate (3.74 kg, 16.70 mol, 0.97 equiv), and 2-MeTHF (10 L, 10 V). This was maintained at 10-25° C. for 1 hour. To this was then charged (R)-2-methyloxirane (Compound 3a) (1.00 kg, 17.22 mol, 1 equiv) and the resulting mixture was maintained at 10-25° C. for 0.5 hours. To this was charged NMP (10 L, 10 V) and the resulting mixture was maintained at 10-25° C. for 10 minutes. The reaction mixture was then passed through a flow reactor (143° C., residence time=30 minutes) at which point GO monitoring showed reaction completion. This reaction mixture afforded crude ethyl (1S,2S)-2-methylcyclopropane-1-carboxylate (Compound 3c, Table 11) which was used directly in the next step.

TABLE 11
GC Method for Part 1 of Example 4

| Column | DB-624 (30 m × 0.32 mm × 1.8 μm) PN 123-1334 | | | |
|---|---|---|---|---|
| Detector | Temperature | 260° C. | | |
| parameters | Flow H2 | 30 mL/min | | |
| | Air flow | 400 mL/min | | |
| | Makeup (N2) | 25 mL/min | | |
| Injector | Temperature | 200° C. | | |
| parameters | Split ratio | 10:1 | | |
| Carrier gas | N2 | | | |
| | Flow | 2 mL/min | | |
| | Mode | Constant Flow | | |
| Oven | Time | Rate | Temperature | Hold |
| parameters | [min] | [° C./min] | [° C.] | [min] |
| | 0.0 | — | 40 | 2 |
| | | 30 | 120 | 0 |
| | | 2 | 130 | 0 |
| | | 40 | 240 | 7 |

| | |
|---|---|
| Retention | (R)-2-methyloxirane: 3.1 min |
| time | ethyl (1S,2S)-2-methylcyclopropane-1-carboxylate: 8.0 min |

HRMS (ESI+)

Calculated for C₇H₁₃O₂ (M+H⁺): 129.09155

Found: 129.1

Part 2—Synthesis of Compound 3—(1S,2S)-2-methylcyclopropane-1-carboxylic acid.

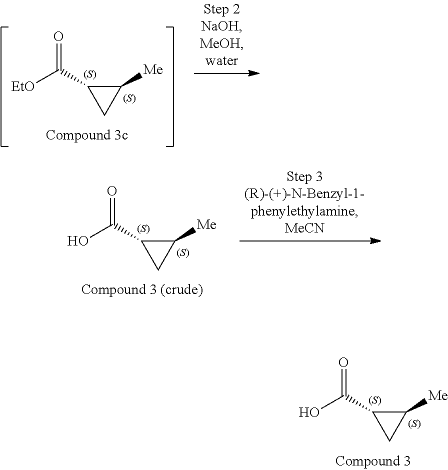

To the crude ethyl (1 S,2S)-2-methylcyclopropane-1-carboxylate (Compound 3) reaction mixture (having started with (R)-2-methyloxirane (Compound 3a) (1.00 kg, 17.22 mol, 1 equiv)) was charged NaOH (2.07 kg, 51.73 mol, 3 equiv), water (3 L, 3 V), and MeOH (3 L, 3 V). The resulting reaction mixture was heated to, and maintained at, 40° C. for 14 hours at which point GC monitoring showed reaction completion.

The reaction mixture was concentrated (40° C.) until no more distillate was observed (˜20 L, ˜20 V final volume). This residue was cooled to, and maintained at, ≤30° C. and to this was charged water (10 L, 10 V). The pH was adjusted to 1 with concentrated HCl (required ˜6 L). The resulting mixture was extracted with MTBE (10 L×3, 10 V×3). The combined organic phases were washed with brine (10 L, 10 V), dried over anhydrous MgSO₄, and filtered. The filtrate was concentrated (30° C.) to a final volume of ˜12 L (−12 V). To this was charged dicyclohexylamine (2.64 kg, 14.56 mol, 0.84 equiv) and the resulting mixture was maintained at room temperature for 12 hours. This was then filtered, washing the cake with MTBE (1 L, 1 V). The cake was dissolved in water (20 L, 20 V) and the pH was adjusted to 1 with concentrated HCl (required ˜1 L). To this was then charged MTBE (10 L, 10 V) and the biphasic mixture was filtered, washing the cake with MTBE (2 L, 2 V). The phases were separated and the aqueous phase extracted with MTBE (10 L, 10 V). The combined organic phases were washed with brine (10 L, 10 V), dried over anhydrous MgSO₄, filtered, and concentrated (30° C.) to afford crude (1S,2S)-2-methylcyclopropane-1-carboxylic acid (Compound 3) (711.5 g, 41% yield from (R)-2-methyloxirane (1)) as a yellow oil.

The crude (1S,2S)-2-methylcyclopropane-1-carboxylic acid (Compound 3) was dissolved in MeCN (9 L, 9 V) and to this was charged (R)-(+)-N-Benzyl-1-phenylethylamine (1.50 kg, 1 equiv). The resulting mixture was heated to, and maintained at, 40° C. for 1 hour. This was then cooled to, and maintained at, 20° C. for 2 hours. The resulting mixture was filtered, washing the cake with MeCN (2 L, 2 V). The cake was dried and then charged into a pre-cooled (5-10° C.) solution of NaOH (200 g) and water (1.3 kg). The resulting mixture was extracted with MTBE (2.6 L×3, 2.6 V×3). The aqueous phases was pH-adjusted to 1 with concentrated 3 M HCl and then extracted with MTBE (2.6 L×3, 2.6 V×3). The combined organic phases were dried over anhydrous MgSO₄, filtered, and concentrated (40° C.) to afford (1S,2S)-2-methylcyclopropane-1-carboxylic acid (Compound 3) (397.4 g, 56% yield, Table 12).

TABLE 12
GC Method for Part 2 of Example 4

| Column | DB-624 (30 m × 0.32 mm × 1.8 μm) PN 123-1334 | | | |
|---|---|---|---|---|
| Detector | Temperature | 260° C. | | |
| parameters | Flow H2 | 30 mL/min | | |
| | Air flow | 400 mL/min | | |
| | Makeup (N2) | 25 mL/min | | |
| Injector | Temperature | 200° C. | | |
| parameters | Split ratio | 10:1 | | |
| Carrier gas | N2 | | | |
| | Flow | 4 mL/min | | |
| | Mode | Constant Flow | | |
| Oven | Time | Rate | Temperature | Hold |
| parameters | [min] | [° C./min] | [° C.] | [min] |
| | 0.0 | — | 40 | 2 |
| | | 30 | 120 | 0 |
| | | 5 | 150 | 0 |
| | | 40 | 240 | 7 |

| | |
|---|---|
| Retention | ethyl (1S,2S)-2-methylcyclopropane-1-carboxylate: 6.3 min |
| time | (1S,2S)-2-methylcyclopropane-1-carboxylic acid: 6.9 min |

LRMS (ESI−)

Calculated for C₅H₇O₂ (M−H⁺): 99.04460

Found: 99.1

¹H NMR (400 MHz, CDCl₃, 25° C.)

δ 11.43 (br s, 1H), 1.49-1.43 (m, 1H), 1.35-1.30 (m, 1H), 1.25-1.22 (m, 1H), 1.12 (d, J=6.4 Hz, 3H), 0.77-0.73 (m, 1H).

### Example 5—Synthetic Procedure for Compound 6a—(1²M)-(S)-(1-ethyl-3-(3-hydroxy-2,2-dimethylpropyl)-2-(2-(1-methoxyethyl)-5-(4-methylpiperazin-1-yl)pyridin-3-yl)-1H-indol-5-yl)boronic acid

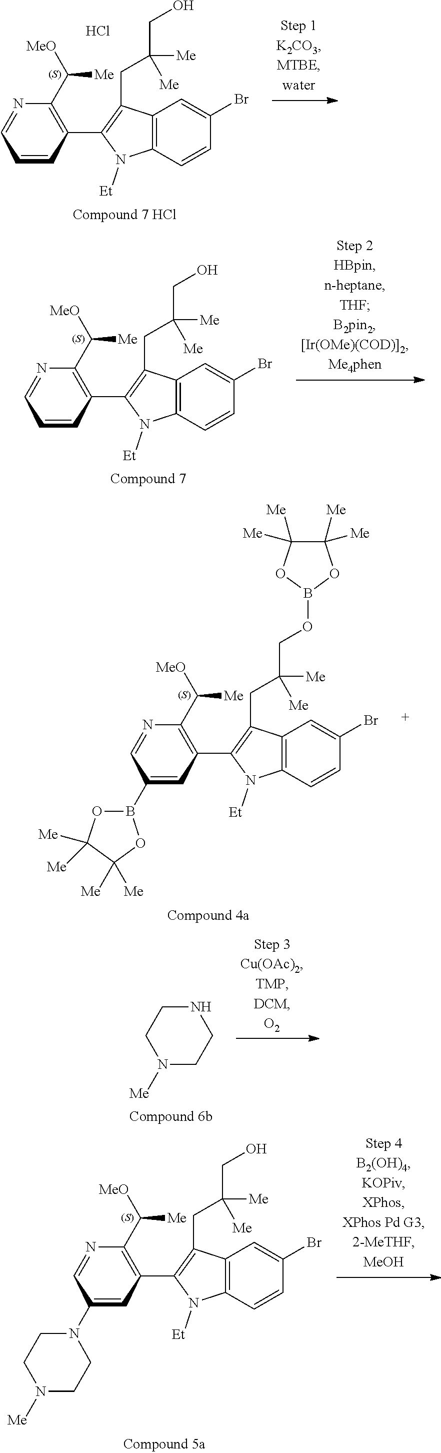

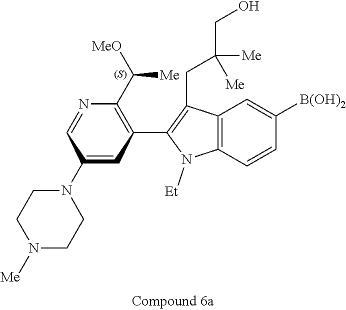

Part 1—Synthesis of Compound 7—(1²M)-(S)-3-(5-bromo-1-ethyl-2-(2-(1-methoxyethyl)pyridin-3-yl)-1H-indol-3-yl)-2,2-dimethylpropan-1-ol.

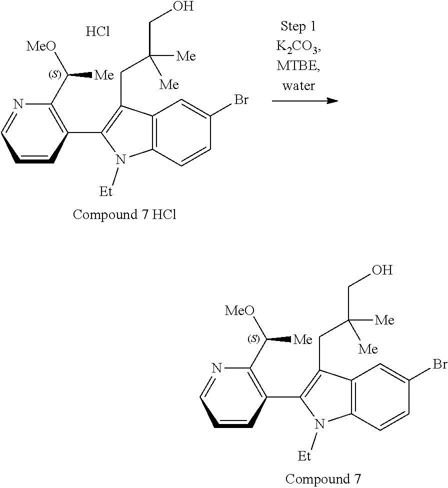

To a reactor was charged water (19 L, 3 V), MTBE (51 L, 8 V), (1²M)-(S)-3-(5-bromo-1-ethyl-2-(2-(1-methoxyethyl)pyridin-3-yl)-1H-indol-3-yl)-2,2-dimethylpropan-1-ol hydrochloride (Compound 7 HCl) (6.35 kg, 13.18 mol, equiv), and K₂CO₃ (1.27 kg, 9.19 mol, 0.7 equiv). The resulting mixture was maintained at 15-25° C. for 30 minutes.

The phases were separated. The organic phase was washed with water (19 L, 3 V), combined with the organic phase of another reaction of the same scale, and concentrated to ˜25 L (˜2 V). This was solvent-exchanged to n-heptane (63 L×2, 5 V×2, concentrating to 25 L, 2 V). The resulting mixture was filtered, washing the cake with n-heptane (12 L, 1 V). The cake was dried to afford (1²M)-(S)-3-(5-bromo-1-ethyl-2-(2-(1-methoxyethyl)pyridin-3-yl)-1H-indol-3-yl)-2,2-dimethylpropan-1-ol (Compound 7) (11.3 kg, 96% yield, Table 13).

TABLE 13
HPLC Method for Part 1 of Example 5

| Column: | XBridge C18 (4.6 mm × 150 mm, 3.5 μm) | | |
|---|---|---|---|
| Mobile Phase: | A: 10 mM NH₄OAc in water | | |
| | B: MeCN | | |
| Gradient: | Time (min) | A % | B % |
| | 0.0 | 55 | 45 |
| | 11.0 | 5 | 95 |
| | 19.0 | 5 | 95 |
| | 20.0 | 55 | 45 |
| | 26.0 | 55 | 45 |

| | |
|---|---|
| Flow Rate: | 0.8 mL/min |
| UV Detector Wavelength: | 230 nm |
| Column Temperature: | 20° C. |
| Retention Times: | (1²M)-(S)-3-(5-bromo-1-ethyl-2-(2-(1-methoxyethyl)pyridin-3- |
| | yl)-1H-indol-3-yl)-2,2-dimethylpropan-1-ol: 8.3 min |

LRMS (ESI+)

Calculated for C₂₃H₃₀BrN₂O₂ (M+H⁺): 445.14907

Found: 445.2

¹H NMR (400 MHz, CDCl₃, 25° C.)

δ 8.82 (dd, J=4.8, 1.8 Hz, 1H), 7.89 (d, J=1.9 Hz, 1H), 7.69 (dd, J=7.7, 1.8 Hz, 1H), 7.41-7.30 (m, 2H), 7.24 (d, J=8.6 Hz, 1H), 4.10 (q, J=6.2 Hz, 1H), 4.06-3.93 (m, 1H), 3.93-3.80 (m, 1H), 3.33-3.17 (m, 2H), 3.07 (s, 3H), 2.72 (d, J=14.2 Hz, 1H), 2.24 (d, J=14.3 Hz, 1H), 1.47 (d, J=6.3 Hz, 3H), 1.29 (s, 1H), 1.18 (t, J=7.2 Hz, 3H), 0.77 (s, 6H).

Part 2a—Synthesis of Compound Compound 4a—(1²M)-(S)-5-bromo-3-(2,2-dimethyl-3-((4,4,5,5-tetramethyl-1,3,2-dioxaborolan-2-yl)oxy)propyl)-1-ethyl-2-(2-(1-methoxyethyl)-5-(4,4,5,5-tetramethyl-1,3,2-dioxaborolan-2-yl)pyridin-3-yl)-1H-indole.

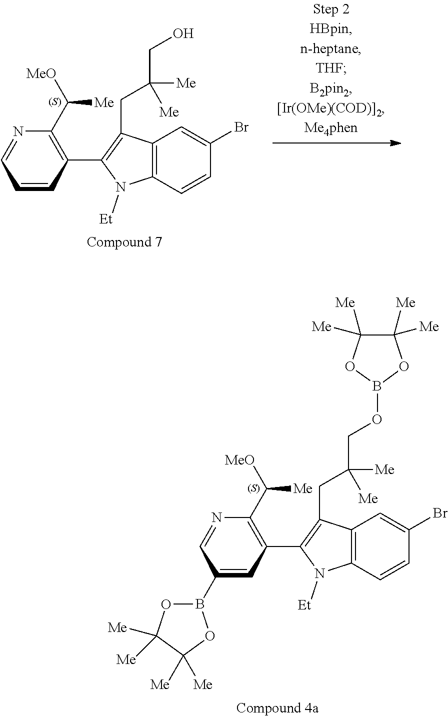

To a reactor was charged n-heptane (17.7 L, 3.3 V), THE (9.1 L, 1.7 V), and (1²M)-(S)-3-(5-bromo-1-ethyl-2-(2-(1-methoxyethyl)pyridin-3-yl)-1H-indol-3-yl)-2,2-dimethylpropan-1-ol (Compound 7) (5.37 kg, 12.06 mol, 1 equiv). To this was charged HBpin (2.32 kg, 18.13 mol, 1.5 equiv) over 2.5 hours. The resulting mixture was heated to, and maintained at, 50° C. for 4 hours at which point it was cooled to 25° C.

To the reaction mixture was charged B₂pin₂ (3.67 kg, 14.47 mol 1.2 equiv), Me₄phen (22.8 g, 96.48 mmol, 0.008 equiv), and [Ir(OMe)(COD)]₂ (16.0 g, 24.14 mmol, 0.002 equiv). The resulting mixture was heated to, and maintained at, 50° C. for 14 hours at which point HPLC monitoring showed reaction completion.

The reaction mixture was cooled to, and maintained at, 15° C. for 1 hour and then concentrated to ˜13.4 L (˜2.5 V). This was solvent-exchanged to n-heptane (16 L×2, 3 V×2, concentrating to ˜13.4 L, ˜2.5 V). The resulting mixture was cooled to, and maintained at, 10-15° C. for 15 hours. This was then filtered, washing the cake with n-heptane (5 L, 1 V). The cake was dried to afford (1²M)-(S)-5-bromo-3-(2,2-dimethyl-3-((4,4,5,5-tetramethyl-1,3,2-dioxaborolan-2-yl)oxy)propyl)-1-ethyl-2-(2-(1-methoxyethyl)-5-(4,4,5,5-tetramethyl-1,3,2-dioxaborolan-2-yl)pyridin-3-yl)-1 H-indole (Compound 4a) (8.37 kg, 98.7% a/a purity 91.5% w/w assay, 91% yield, Table 14) as a light brown solid.

Part 2b—Alternative Synthesis of Compound Compound 4a—(1²M)-(S)-5-bromo-3-(2,2-dimethyl-3-((4,4,5,5-tetramethyl-1,3,2-dioxaborolan-2-yl)oxy)propyl)-1-ethyl-2-(2-(1-methoxyethyl)-5-(4,4,5,5-tetramethyl-1,3,2-dioxaborolan-2-yl)pyridin-3-yl)-1H-indole.

To a reactor was charged water (601 kg, 3V), MTBE (1186.9 kg, 8V), (1²M)-(S)-3-(5-bromo-1-ethyl-2-(2-(1-methoxyethyl)pyridin-3-yl)-1H-indol-3-yl)-2,2-dimethylpropan-1-ol (Compound 7) HCl salt (200 kg, 1.0 eq.), and K₂CO₃ (37.29 kg, 0.65 eq.). The solution was stirred for 0.5 hours at 20° C. and then left unagitated for 0.5 hours. The phases were separated, and the organic phase was washed with water (600.5 kg, 3 V). The MTBE solution was concentrated under reduced pressure until the residue volume was around 400 L. To the solution was charged THF(354.3 kg, 2 V). The resultant solution was concentrated under reduced pressure until the residue volume was around 400 L. The solvent swap was repeated two more times. To the solution of Compound 7 in THE was charged n-heptane (407.7 kg, 3.05 V). N₂ was bubbled under the surface of the solution for 1 hour. HBpin (59.5 kg, 1.1 eq.) was charged dropwise into the reactor under N₂ atmosphere at 20° C. The reaction mixture was heated at 30° C. for 2 hours and cooled to 20° C. B₂pin₂ (126 kg, 1.2 eq.) and Me₄phen (779 g, 0.8 mol %) were charged into reactor under N₂ atmosphere. N₂ was bubbled under surface of the solution for 1 hour. [IrOMe(COD)]₂ (545 g, 0.2 mol %) was added into reactor under N₂ atmosphere. The reaction mixture was heated at 45° C. for 6 hours and cooled to 20° C. After the reaction, EtOH (28.6 kg, 1.5 eq.) was added to the reaction mixture and stirred at 20° C. for 17 hours. The reaction mixture was concentrated under reduced pressure until the residue volume was around 400 L. The suspension was charged with n-heptane (273.10 kg, 2 V). The resultant suspension was concentrated under reduced pressure until the residue volume was around 400 L. The solvent swap was repeated one more time. The resulting suspension was cooled to 5° C., stirred for 20 hours, and filtered to give a wet cake, which was washed with n-heptane (14 kg). The washed wet cake was dried below 45° C. for 16 hours to give 277.74 kg of (1²M)-(S)-5-bromo-3-(2,2-dimethyl-3-((4,4,5,5-tetramethyl-1,3,2-dioxaborolan-2-yl)oxy)propyl)-1-ethyl-2-(2-(1-methoxyethyl)-5-(4,4,5,5-tetramethyl-1,3,2-dioxaborolan-2-yl)pyridin-3-yl)-1 H-indole (Compound 4a) as an off-white solid with 97.0% purity, 96.4% assay, and a yield of 92.5% as an off-white solid.

TABLE 14
HPLC Method for Parts 2a and 2b of Example 5

| Column: | XBridge C18 (4.6 mm × 150 mm, 3.5 μm) | | |
|---|---|---|---|
| Mobile Phase: | A: 0.05% TFA in water | | |
| | B: 0.05% TFA in MeCN | | |
| Gradient: | Time (min) | A % | B % |
| | 0.0 | 95 | 5 |
| | 8.0 | 0 | 100 |
| | 12.0 | 0 | 100 |
| | 12.1 | 95 | 5 |
| | 16.0 | 95 | 5 |

| | |
|---|---|
| Flow Rate: | 1.0 mL/min |
| UV Detector Wavelength: | 210 nm |
| Column Temperature: | 30° C. |
| Retention Times: | (1²M)-(S)-3-(5-bromo-1-ethyl-2-(2-(1-methoxyethyl)pyridin-3- |
| | yl)-1H-indol-3-yl)-2,2-dimethylpropan-1-ol: 6.97 min |
| | (1²M)-(S)-5-bromo-3-(2,2-dimethyl-3-((4,4,5,5-tetramethyl- |
| | 1,3,2-dioxaborolan-2-yl)oxy)propyl)-1-ethyl-2-(2-(1- |
| | methoxyethyl)-5-(4,4,5,5-tetramethyl-1,3,2-dioxaborolan-2- |
| | yl)pyridin-3-yl)-1H-indole: 6.54 min |

LRMS (ESI+)

Calculated for C₂₃H₃₁BBrN₂O₄ (M+H⁺) (free alcohol, free boronic acid): 489.15603

Found: 489.2

¹H NMR (400 MHz, CDCl₃, 25° C.) δ 9.11 (d, J=1.7 Hz, 1H), 8.03 (d, J=1.8 Hz, 1H), 7.87 (d, J=1.9 Hz, 1H), 7.31 (dd, J=8.6, 1.9 Hz, 1H), 7.22 (d, J=8.7 Hz, 1H), 4.08 (q, J=6.3 Hz, 1H), 4.02-3.91 (m, 1H), 3.90-3.80 (m, 1H), 3.55-3.43 (m, 2H), 3.09 (s, 3H), 2.76 (d, J=14.2 Hz, 1H), 2.14 (d, J=14.2 Hz, 1H), 1.43 (d, J=6.3 Hz, 3H), 1.35 (d, J=2.9 Hz, 12H), 1.25 (s, 12H), 1.17 (t, J=7.2 Hz, 3H), 0.76 (s, 3H), 0.68 (s, 3H).

Part 3—Synthesis of Compound 5a—(1²M)-(S)-3-(5-bromo-1-ethyl-2-(2-(1-methoxyethyl)-5-(4-methylpiperazin-1-yl)pyridin-3-yl)-1H-indol-3-yl)-2,2-dimethylpropan-1-ol.

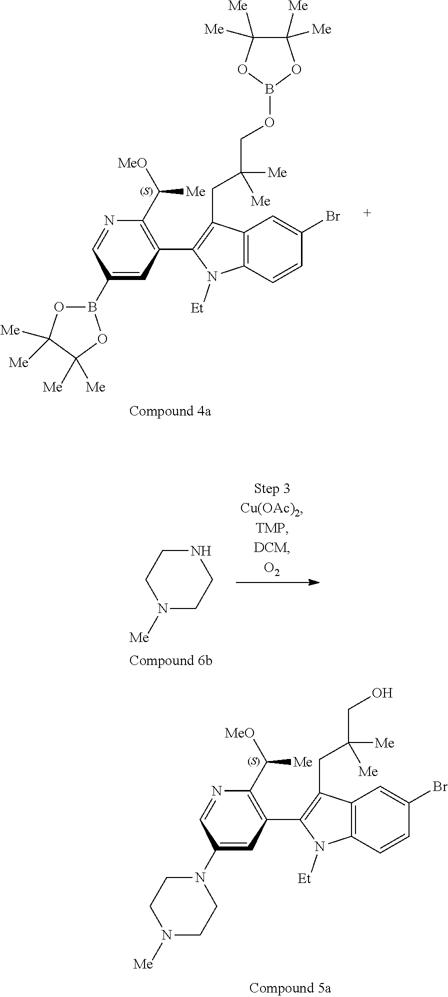

To a reactor was charged (1²M)-(S)-5-bromo-3-(2,2-dimethyl-3-((4,4,5,5-tetramethyl-1,3,2-dioxaborolan-2-yl)oxy)propyl)-1-ethyl-2-(2-(1-methoxyethyl)-5-(4,4,5,5-tetramethyl-1,3,2-dioxaborolan-2-yl)pyridin-3-yl)-1H-indole (Compound 4a) (11.88 kg, 17.04 mol, 1 equiv), 1-methylpiperazine (Compound 6b) (17.11 kg, 170.82 mol, 10 equiv), Cu(OAc)₂ (19.01 kg, 104.66 mol, 6 equiv), TMP (8.91 kg, 63.01 mol, 3.7 equiv), and DCM (238 L, 20 V). The resulting mixture was maintained at 20-25° C. and bubbled with 21% O₂ in N₂ for 16 hours at which point HPLC monitoring showed reaction completion.

The reaction mixture was concentrated to 119 L (10 V). The resulting mixture was charged into a mixture of 28% w/w aqueous NH₃ (35.6 L, 3 V) and water (71.3 L, 6 V). This biphasic mixture was filtered and the filtrate phases separated. The organic phase was washed with 28% w/w aqueous NH₃ (35.6 L, 3 V), washed with 0.1 M aqueous EDTA (35.6 L, 3 V), and concentrated to 17.8 L (1.5 V). To this residue was charged 2-MeTHF (35.6 L, 3 V) and water (11.9 L, 1 V). The pH was adjusted to 1-2 with 6 M aqueous HCl (required 23.5 L). The phases were separated and the aqueous phase extracted with 2-MeTHF (35.6 L×2, 3 V×2). To the aqueous phase was charged DCM (35.6 L, 3 V). The pH was adjusted to 8-9 with 30% w/w aqueous NaOH (required 4.5 L). The phases were separated and the organic phase concentrated to 12 L (1 V). To this was charged n-heptane (65.3 L, 5.5 V). The resulting mixture was maintained at 35° C. for 3 hours and then cooled to, and maintained at, −5° C. for 12 hours. This mixture was filtered, washing the cake with n-heptane (5.9 L, 0.5 V). The cake was dried to afford (1²M)-(S)-3-(5-bromo-1-ethyl-2-(2-(1-methoxyethyl)-5-(4-methylpiperazin-1-yl)pyridin-3-yl)-1H-indol-3-yl)-2,2-dimethylpropan-1-ol (Compound 5a) (7.28 kg, 98.7% a/a purity, 91.7% w/w assay, 72% yield) as an off-white solid.

Part 4a—Alternative Synthesis of Compound 5a—(1²M)-(S)-3-(5-bromo-1-ethyl-2-(2-(1-methoxyethyl)-5-(4-methylpiperazin-1-yl)pyridin-3-yl)-1H-indol-3-yl)-2,2-dimethylpropan-1-ol.

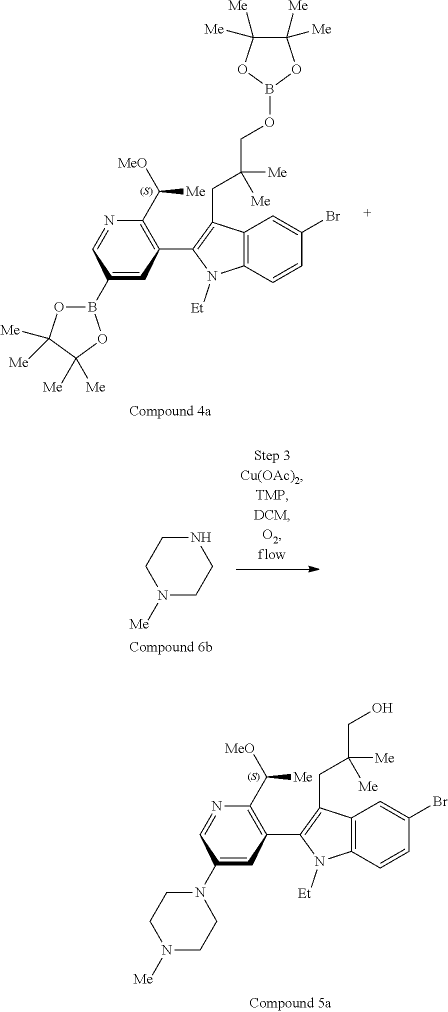

A solution of (1²M)-(S)-5-bromo-3-(2,2-dimethyl-3-((4,4,5,5-tetramethyl-1,3,2-dioxaborolan-2-yl)oxy)propyl)-1-ethyl-2-(2-(1-methoxyethyl)-5-(4,4,5,5-tetramethyl-1,3,2-dioxaborolan-2-yl)pyridin-3-yl)-1 H-indole (Compound 4a) (6.5 kg, 9.32 mol, 1 equiv) in DCM (65 L, 10 V) and a separate solution of 1-methylpiperazine (Compound 6b) (12.53 kg, 125.09 mol, 13.4 equiv), Cu(OAc)₂ (11.36 kg, 62.54 mol, 6.7 equiv), and TMP (8.84 kg, 62.58 mol, 6.7 equiv) in DCM (65 L, 10 V) were passed through a flow reactor (35-45° C., residence time=1.5 hours) at which point HPLC monitoring showed reaction completion.

The reaction mixture was combined with another reaction mixture (total input of 13.2 kg, 18.93 mol, 1 equiv (1²M)-(S)-5-bromo-3-(2,2-dimethyl-3-((4,4,5,5-tetramethyl-1,3,2-dioxaborolan-2-yl)oxy)propyl)-1-ethyl-2-(2-(1-methoxyethyl)-5-(4,4,5,5-tetramethyl-1,3,2-dioxaborolan-2-yl)pyridin-3-yl)-1H-indole (Compound 4a)).

The reaction mixture was concentrated to 132 L (10 V). The resulting mixture was charged into a mixture of 28% w/w aqueous NH₃ (43.6 L, 3.3 V) and water (87.1 L, 6.6 V). This biphasic mixture was filtered and the filtrate phases separated. The organic phase was washed with 28% w/w aqueous NH₃ (43.6 L, 3.3 V), washed with 0.1 M aqueous EDTA (43.6 L, 3.3 V), and concentrated to ˜20 L (˜1.5 V). To this residue was charged 2-MeTHF (39.6 L, 3 V) and water (13 L, 1 V). The pH was adjusted to 1-2 with 6 M aqueous HCl. The phases were separated and the aqueous phase extracted with 2-MeTHF (39.6 L×2, 3 V×2). To the aqueous phase was charged DCM (39.6 L, 3 V). The pH was adjusted to 8-9 with $ M aqueous NaOH.

The phases were separated and the organic phase concentrated to ˜20 L (˜1.5 V). To this was charged n-heptane (73 L, 5.5 V) over 2 hours. The resulting mixture was maintained at 35° C. for 3 hours and then cooled to, and maintained at, 0° C. for 15 hours. This mixture was filtered, washing the cake with n-heptane (7 L, 0.5 V). The cake was dried to afford (1²M)-(S)-3-(5-bromo-1-ethyl-2-(2-(1-methoxyethyl)-5-(4-methylpiperazin-1-yl)pyridin-3-yl)-1H-indol-3-yl)-2,2-dimethylpropan-1-ol (Compound 5a) (7.2 kg, 98.2% a/a purity, 83.4% w/w assay, 58% yield, Table 15) as an off-white solid.

Part 4b—Alternative Synthesis of Compound 5a—(1²M)-(S)-3-(5-bromo-1-ethyl-2-(2-(1-methoxyethyl)-5-(4-methylpiperazin-1-yl)pyridin-3-yl)-1H-indol-3-yl)-2,2-dimethylpropan-1-ol.

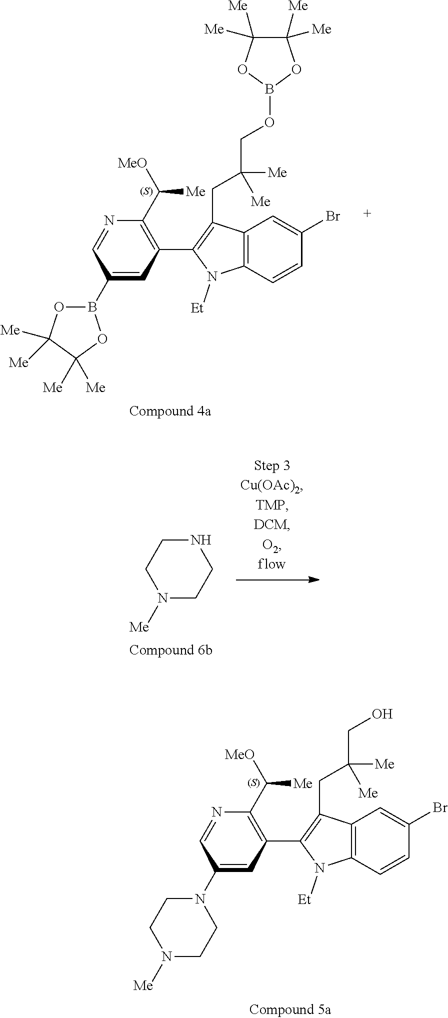

A reactor was charged with DCM (2493 kg, 16 V), Cu(OAc)₂ (143 kg, 5.0 eq.), N-methyl piperazine (160 kg, 10 eq.) and TMP (83 kg, 3.7 eq.) at 23° C. The solution was stirred for 0.5 hours at 23° C. A mixture of N₂—O₂ (21% O₂) gases was bubbled under the surface of the reaction mixture at 23° C. for 2 hours. Compound 4a (114.5 kg, 1.0 eq) was charged into reactor at 23° C. The mixture of N₂—O₂ (21% 02) was bubbled under the surface of the reaction mixture at 23° C. for 6 hours. After the reaction was completed, the reaction mixture was concentrated under reduced pressure until the residue volume was around 1100 L. In another reactor, water (306 kg, 6 V) and 28% ammonium hydroxide (306 kg, 3 V) were charged with the concentrated reaction mixture at 20° C. The phases were separated. The organic layer was collected and washed with EDTA-Na₂ aqueous solution (340 kg (0.1 N), 3 V) at 20° C. to purge copper salt. The DCM solution was concentrated until the residue volume was around 220 L.

Another two batches of Chan-Lam (net input of Compound 4a for these two batches was 400 kg) were carried out and washed by NH₃, EDTA to give DCM solutions; three batches of DCM solution of crude Compound 5a were combined to perform acidic MeTHF washing, freebasing, DCM extraction, water washing, and ACN crystallization.

To the reactor was charged 2-MeTHF (1333 kg, 3 V) and water (510 kg, 1 V). The bi-phasic solution was adjusted to a pH of 1.29 with 6N HCl. The bi-phasic solution was stirred at 20° C. for 30 min. Phases were separated. The aqueous phase was collected and washed twice with 2-MeTHF (1340 kg×2, 3 V×2). To the aqueous layer, DCM (2029 kg, 3 V) and aq. NaOH (30% W/W) were added to adjust the pH to 8.39. The DCM phase was separated and washed with water (1533 kg, 3 V). The DCM solution was concentrated under vacuum until the residue volume was 1250 L. The resultant solution was swapped with ACN (835.9 kg×3, V×3) three times. The resultant solution was heated to 75° C. and stirred at 75° C. until all solids dissolved. The solution was cooled to 60° C. slowly over 2 hours. Seeds of Compound 5a were added into the reactor at 60° C. The suspension was stirred at 60.° C. for 2 hours and cooled to 25° C. over 5 hours, and stirred at 25° C. for 3 hours. The suspension was concentrated under vacuum until the residue volume was around 230 L. The suspension was further cooled to 5° C. over 4 hours. The resulting suspension was stirred at 5° C. for 12 hours. The suspension was filtered and washed with pre-cooled ACN (481 kg). The wet cake was dried under vacuum at 45° C. for 15 hours to give 307.6 kg of Compound 5a with 99.3% purity and 98.0% assay in yield of 75.8% as an off-white solid.

TABLE 15
HPLC Method for Parts 3, 4a, and 4b of Example 5

| Column: | XBridge C18 (4.6 mm × 150 mm, 3.5 μm) | | |
|---|---|---|---|
| Mobile Phase: | A: 0.05% TFA in water | | |
| | B: 0.05% TFA in MeCN | | |
| Gradient: | Time (min) | A % | B % |
| | 0.0 | 95 | 5 |
| | 8.0 | 0 | 95 |
| | 12.0 | 0 | 95 |
| | 13.0 | 95 | 5 |
| | 18.0 | 95 | 5 |

| | |
|---|---|
| Flow Rate: | 1.0 mL/min |
| UV Detector Wavelength: | 210 nm |
| Column Temperature: | 30° C. |
| Retention Times: | (1²M)-(S)-5-bromo-3-(2,2-dimethyl-3-((4,4,5,5-tetramethyl- |
| | 1,3,2-dioxaborolan-2-yl)oxy)propyl)-1-ethyl-2-(2-(1- |
| | methoxyethyl)-5-(4,4,5,5-tetramethyl-1,3,2-dioxaborolan-2- |
| | yl)pyridin-3-yl)-1H-indole: 7.3 min |
| | (1²M)-(S)-3-(5-bromo-1-ethyl-2-(2-(1-methoxyethyl)-5-(4- |
| | methylpiperazin-1-yl)pyridin-3-yl)-1H-indol-3-yl)-2,2- |
| | dimethylpropan-1-ol: 6.6 min |

### LRMS (ESI+)

Calculated for C₂₈H₄₀BrN₄O₂ (M+H⁺): 543.23346

Found: 543.2

¹H NMR (400 MHz, CDCl₃, 25° C.)

δ 8.51 (d, J=2.9 Hz, 1H), 7.88 (d, J=1.8 Hz, 1H), 7.32 (dd, J=8.7, 1.9 Hz, 1H), 7.23 (d, J=8.6 Hz, 1H), 7.11 (d, J=3.0 Hz, 1H), 4.06-3.85 (m, 3H), 3.35-3.17 (m, 6H), 3.05 (s, 3H), 2.70 (d, J=14.2 Hz, 1H), 2.60 (t, J=5.1 Hz, 4H), 2.37 (s, 3H), 2.27 (d, J=14.2 Hz, 1H), 1.44 (d, J=6.2 Hz, 3H), 1.35 (s, 1H), 1.20 (t, J=7.2 Hz, 3H), 0.78 (s, 6H).

Part 5a—Synthesis of Compound 6a—(1²M)-(S)-(1-ethyl-3-(3-hydroxy-2,2-dimethylpropyl)-2-(2-(1-methoxyethyl)-5-(4-methylpiperazin-1-yl)pyridin-3-yl)-1H-indol-5-yl)boronic acid.

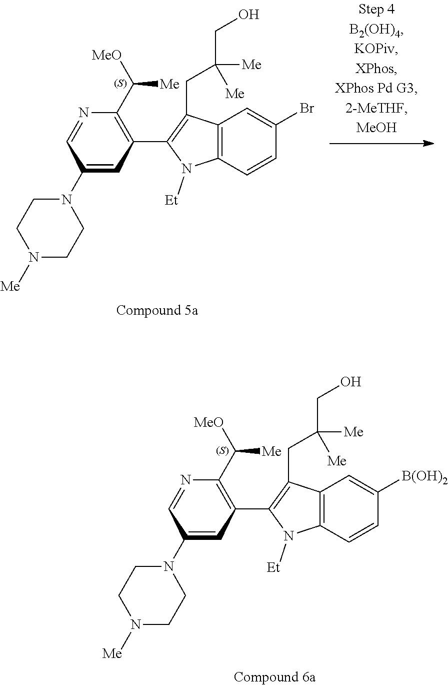

To a reactor was charged 2-MeTHF (18.7 L, 6.6 V), MeOH (6.2 L, 2.2 V), KOPiv (1.60 kg, 11.41 mol, 2.2 equiv), (1²M)-(S)-3-(5-bromo-1-ethyl-2-(2-(1-methoxyethyl)-5-(4-methylpiperazin-1-yl)pyridin-3-yl)-1H-indol-3-yl)-2,2-dimethylpropan-1-ol (Compound 5a) (2.85 kg, 5.25 mol, 1 equiv), XPhos (54.3 g, 113.90 mmol, 0.02 equiv), XPhos Pd G3 (48.2 g, 56.94 mmol, 0.01 equiv), and B₂(OH)₄ (307 g, 3.42 mol, 0.7 equiv). The resulting mixture was heated to, and maintained at, 30° C. for 2 hours. To this was charged additional B₂(OH)₄ (307 g, 3.42 mol, 0.7 equiv) and the resulting mixture was maintained at 30° C. for 2 hours at which point HPLC monitoring showed reaction completion.

The reaction mixture was concentrated to ˜11 L (˜4 V) and then cooled to 20° C. To this was charged water (3.1 L, 1 V). The resulting mixture was maintained at 20° C. for 12 hours at which point it was filtered, washing the cake with water (6.2 L, 2 V). The cake was combined with the cake from another reaction of the same scale and then slurried in MeOH (37.2 L, 6.5 V) and water (12.4 L, 2.2 V) at 20° C. for 12 hours. The resulting mixture was then filtered, washing the cake with a mixture of MeOH:water (3:1, v/v, 6 L, 1 V). The cake was dried to afford (1²M)-(S)-(1-ethyl-3-(3-hydroxy-2,2-dimethylpropyl)-2-(2-(1-methoxyethyl)-5-(4-methylpiperazin-1-yl)pyridin-3-yl)-1H-indol-5-yl)boronic acid (Compound 6a) (5.19 kg, 97.2% purity, 92.7% w/w assay, 90% yield, Table 16) as an off-white solid.

Part 5b—Alternative Synthesis of Compound 6a—(1²M)-(S)-(1-ethyl-3-(3-hydroxy-2,2-dimethylpropyl)-2-(2-(1-methoxyethyl)-5-(4-methylpiperazin-1-yl)pyridin-3-yl)-1H-indol-5-yl)boronic acid.

To a reactor was charged with (1²M)-(S)-3-(5-bromo-1-ethyl-2-(2-(1-methoxyethyl)-5-(4-methylpiperazin-1-yl)pyridin-3-yl)-1H-indol-3-yl)-2,2-dimethylpropan-1-ol (Compound 5a) (287.6 kg, 529.79 mol, 1 equiv), KOPiv (148 kg, 2025 mol, 2.1 equiv), XPhos (4.0 kg, 8.39 mol, 0.02 equiv), XPhos G3 Pd(3.4 kg, 4.02 mol, 0.01 equiv), and B₂(OH)₄ (71.0 kg, 791 mol, 1.5 equiv) in 2-MeTHF (870 L, 3.03 V). A solution of Compound 5a in 2-MeTHF (870 L, 3.03 V) and MeOH (580 L, 2.02 V) was charged to the above reactor at 30° C. over a period of 1 hour. The resulting mixture was maintained at 30° C. for 3 hours, at which point HPLC monitoring showed reaction completion.

A reactor was charged with water (28 L, 0.1 V). The reaction mixture was concentrated to 987 L (˜3.5 V) and then cooled to 20° C. Water (256 L, 0.9 V) was added to this mixture. The resulting mixture was maintained at 20° C. for 16 hours, at which point it was filtered, and the cake was washed with water (471 L, 1.7 V). The cake was slurried in MeOH (1690 L, 6.0 V) and water (571 L, 2.0 V) at 15° C. for 8 hours. The resulting mixture was then filtered, and the cake was washed with a mixture of MeOH: water (3:1, v/v, 674 L, 2.4 V). The cake was dried to afford (12M)-(S)-(1-ethyl-3-(3-hydroxy-2,2-dimethylpropyl)-2-(2-(1-methoxyethyl)-5-(4-methylpiperazin-1-yl)pyridin-3-yl)-1 H-indol-5-yl)boronic acid (Compound 6a) (250.18 kg, 99.0% purity, 97.5% w/w assay, 94.2% yield) as off-white solid.

TABLE 16
HPLC Method for Parts 5a and 5b of Example 5

| Column: | Express-C18 (4.6 mm × 150 mm, 2.7 μm) | | |
|---|---|---|---|
| Mobile Phase: | A: 10 mM NH₄OAc in water | | |
| | B: MeCN | | |
| Gradient: | Time (min) | A % | B % |
| | 0.0 | 90 | 10 |
| | 15.0 | 60 | 40 |
| | 20.0 | 5 | 95 |
| | 23.0 | 5 | 95 |
| | 23.1 | 30 | 10 |
| | 28.0 | 90 | 10 |

| | |
|---|---|
| Flow Rate: | 1.5 mL/min |
| UV Detector Wavelength: | 235 nm |
| Column Temperature: | 40° C. |
| Retention Times: | (1²M)-(S)-3-(5-bromo-1-ethyl-2-(2-(1-methoxyethyl)-5-(4- |
| | methylpiperazin-1-yl)pyridin-3-yl)-1H-indol-3-yl)-2,2- |
| | dimethylpropan-1-ol: 19.34 min |
| | (1²M)-(S)-(1-ethyl-3-(3-hydroxy-2,2-dimethylpropyl)-2-(2-(1- |
| | methoxyethyl)-5-(4-methylpiperazin-1-yl)pyridin-3-yl)-1H- |
| | indol-5-yl)boronic acid: 12.12 min |

LRMS (ESI+)

Calculated for C₂₈H₄₂BN₄O₄ (M+H⁺): 509.32991

Found: 509.5

¹H NMR (400 MHz, CD₃OD, 25° C.)

δ 8.40 (d, J=2.9 Hz, 1H), 8.06 (s, 1H), 7.53 (s, 1H), 7.45-7.35 (m, 2H), 4.17-4.05 (m, 1H), 4.01 (q, J=6.3 Hz, 1H), 3.92-3.80 (m, 1H), 3.40-3.30 (m, 6H), 3.28 (d, J=12.0, 1H), 3.17 (d, J=12.0 Hz, 1H), 2.99 (s, 3H), 2.80 (d, J=14.0 Hz, 1H), 2.64 (t, J=5.1 Hz, 4H), 2.36 (s, 3H), 2.27 (d, J=14.1 Hz, 1H), 1.40 (d, J=6.3 Hz, 3H), 1.23 (t, J=7.1 Hz, 3H), 0.76 (d, J=23.5 Hz, 6H).

### Example 6. Synthetic Procedure for Compound 9—(S)-1-((S)-3-(4-bromothiazol-2-yl)-2-((1S,2S)-2-methylcyclopropane-1-carboxamido)propanoyl)hexahydropyridazine-3-carboxylic acid

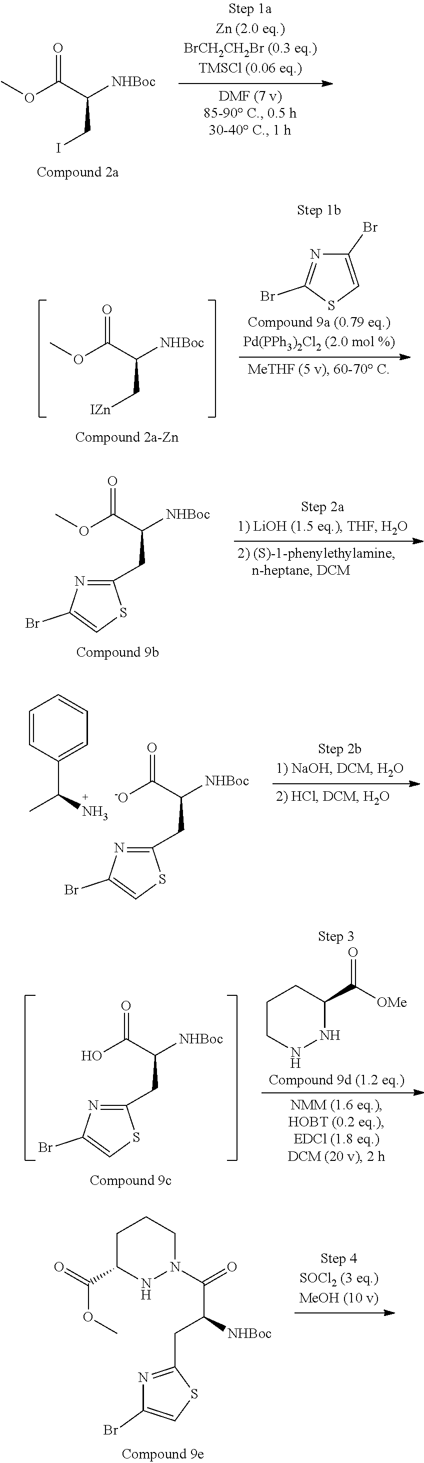

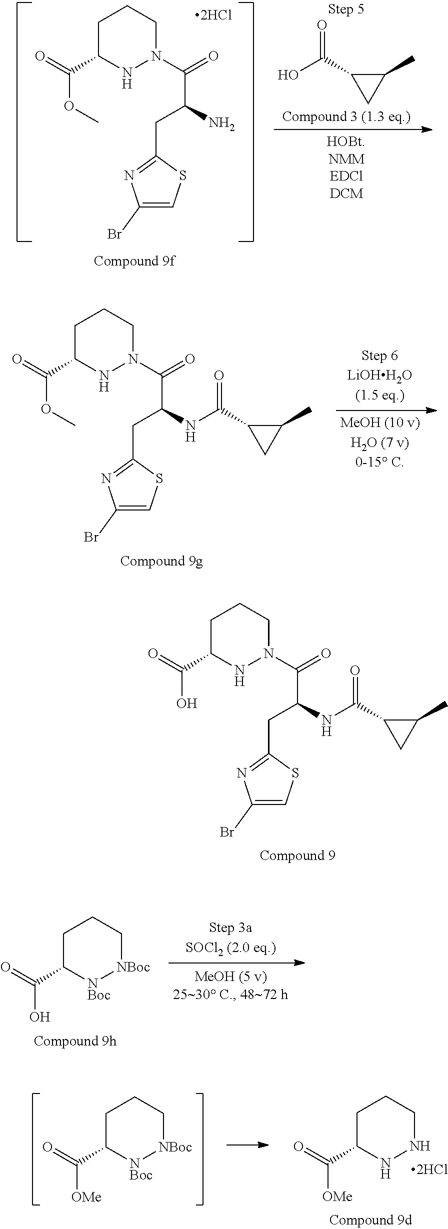

Part 1—Preparation of Compound 9b: methyl (S)-3-(4-bromothiazol-2-yl)-2-((tert-butoxycarbonyl)amino) Propanoate

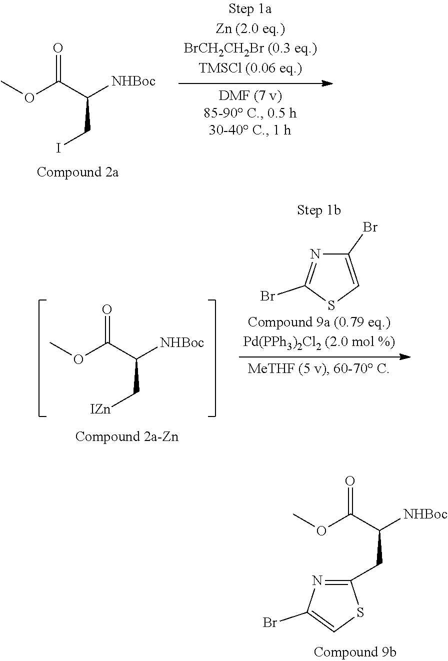

To a reactor 1 was charged DMF (689 kg, 5 vol., the water content was ca. 100 ppm by KF titration) and Zn (57.6 kg, 881.1 mol, 2.0 eq.). Reactor 1 was evacuated and backfilled with Argon (Ar) 3 times, and then bubbled with Ar for 1 h. 1,2-Dibromoethane (24.8 kg, 132.2 mol, 0.3 eq.) was added into reactor 1. The resulting mixture was warmed to 85-95° C. and maintained for 30 min. TMSCI (2.87 kg, 26.4 mol, 0.06 eq.) was added into reactor 1 at 20-30° C. and stirred for 30 min. To a reactor 2 was charged DMF (276 kg, 2 vol., water content by KF titration ca. 100 ppm) and methyl (R)-2-((tert-butoxycarbonyl)amino)-3-iodopropanoate (Compound 2a) (145.0 kg, 440.6 mol, 1.0 eq.). Reactor 2 was evacuated and backfilled with Ar 3 times and then bubbled with Ar for 1 h. The DMF solution of Compound 2a in reactor 2 was added into reactor 1 at 20-3000. The resulting mixture in reactor 1 was warmed to 3040° C. and maintained for another 30 m. To a reactor 3 was charged MeTHF (624 kg, 5 vol.) and 2,4-dibromothiazole (Compound 9a) (96 kg, 359.2 mol, 0.9 eq.). Reactor 3 was evacuated and backfilled with Ar 3 times and then bubbled with Ar for 1 h. Pd(PPh₃)₂Cl₂ (6.2 kg, 8.81 mol, 0.02 eq.) was added into reactor 3. The Reformatsky reagent in reactor 1 was filtered and the filtrate was directly added into the reactor 3 at 20-40° C. The mixture in reactor 3 was warmed to 60-70° C. and maintained for 4 h. A sample was taken for IPC (HPLC: 45.6 A % of Compound 9b was generated). The reaction mixture was concentrated under reduced pressure at 60-70° C. to ca. 300 L (˜2 vol.). MTBE (537 kg, 5 vol.) and 10 wt % aq. NaCl (1450 kg, 10.0 vol.) were added into the mixture at 20-30° C. The mixture was separated and the aqueous phase was extracted with MTBE (537 kg, 5.0 vol.). The MTBE solutions were combined and washed with 10 wt % aq. NaCl (1450 kg×3, 10 vol.×3). The MTBE phase was concentrated under reduced pressure at 35-450C to ca. 200 L (1.5 vol). The resulting solution was subjected to solvent swap with THE two times (645 kg×2, 5 vol.×2) at 35-45° C. A total of 290.4 kg of a THE solution of Compound 9b was obtained with 59.5 A % HPLC purity and 32.8 wt % assay in a 61.4% assay-corrected yield (Table 17). The crude product was taken to the next step without further purification.

TABLE 17
HPLC Method for Compound 9b

| Instrument: | HPLC (e.g., Agilent 1260 series) | |
|---|---|---|
| Column: | Agilent Poroshell 120 EC-C18 (4.6 × 100 mm, 2.7 μm) | |
| Mobile Phase: | A: 0.1% phosphoric acid in water | |
| | B: Acetonitrile | |
| Gradient: | Time (min) | B (%) |
| | 0.00 | 5 |
| | 20.0 | 50 |
| | 25.0 | 90 |
| | 32.0 | 90 |

| | Post time: 3 min |
|---|---|
| Flow Rate | 1.0 mL/min |
| UV Detector Wavelength: | 210 nm |
| Column Temperature: | 30° C. |
| Retention Times: | 2,4-dibromothiazole = 16.2 min; |
| | methyl (R)-2-((tert-butoxycarbonyl)amino)-3- |
| | iodopropanoate ≈ 19.3 min; |
| | methyl (S)-3-(4-bromothiazol-2-yl)-2-((tert- |
| | butoxycarbonyl)amino) propanoate ≈ 20.7 min |

MS (ESI+):

Calculated for C₁₂H₁₇BrN₂O₄S (M+H⁺): 365.01

Found: 365.10

¹H NMR (400 MHz, CDCl₃):

δ 7.12 (s, 1H), 5.47 (d, J=7.2 Hz, 1H), 4.68 (d, J=6.8 Hz, 1H), 3.75 (s, 3H), 3.51 (d, J=5.1 Hz, 2H), 1.43 (s, 9H).

Part 2a—Preparation of Compound 9c: (S)-3-(4-bromothiazol-2-yl)-2-((tert-butoxycarbonyl)amino)propanoic acid

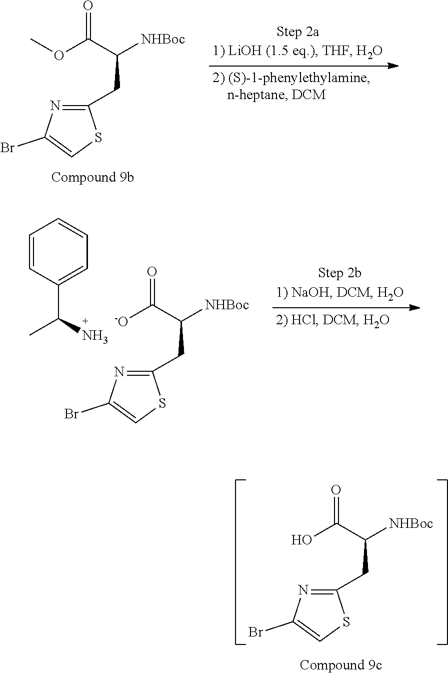

To a reactor was charged (S)-3-(4-bromothiazol-2-yl)-2-((tert-butoxycarbonyl)amino)propanoic acid (Compound 9b) (290.4 kg of THE solution, 59.5 A % purity, 32.8 wt %, 260.8 mol, 1.0 eq.) and THE (847.7 kg, 10.0 vol.). The reactor was evacuated and backfilled with nitrogen 3 times. LiOH·H₂O (16.4 kg, 391.2 mol, 1.5 eq.) in water (667 kg, 7.0 vol.) was added dropwise into the mixture at −2-2° C. The mixture was stirred at 0-5° C. for 3 h followed by sampling for IPC (HPLC purity: 58.3 A % of Compound 9c and 0.3 A % of Compound 9b). The reaction mixture was adjusted to pH 8-9 with 1 M HCl (ca. 100 kg) at 2-10° C. (IT). Water (667 kg, 7.0 vol.) was added into the mixture. The resulting mixture was concentrated under reduced pressure at 30-40° C. until the residual volume reached 1300 L (14.0 vol.). EtOAc (429 kg, 7.0 vol.) was added into the mixture at 15-20° C. and stirred for 30 min. The mixture was filtered and the filtrate was separated to remove the organic layer. The aqueous phase was washed with EtOAc (429 kg×2). The aqueous phase was adjusted to pH 2.8-3.0 with 3 M aq. HCl (ca. 300 kg) at 5-10° C. The aqueous phase was extracted with DCM (633 kg×4). The DCM phases were combined and washed with water (476 kg, 5.0 vol.). The DCM phase was concentrated under reduced pressure at 30-40° C. to ca. 7 vol. (about 660 L). Then (S)-1-Phenylethylamine (44.2 kg, 364.7 mol, 1.4 eq.) was added into the solution at 15-20° C. The mixture was stirred at 15-20° C. for 30 min. n-Heptane (1557 kg, 25 vol.) was added at 15-20° C. (IT) and stirred for 60 min, then stirred at 0-10° C. for another 60 min. The mixture was filtered and the filter cake was rinsed with 2.5:1 (vol/vol.) n-heptane/DCM (183 kg, 2 vol.). The wet filter cake was dried at 40-45° C. under reduced pressure for 12 h. A total of 129.0 kg of the (S)-1-phenylethylamine salt of Compound 9c was obtained as a white solid with 96.7% HPLC purity and 65.3 wt % assay by HPLC. The (S)-1-phenylethylamine salt of Compound 9c was dissolved in water (1684 kg, 20.0 vol.) and DCM (1120 kg, 10.0 vol.) was added. The mixture was adjusted to pH 10-10.5 with 1 M aq. NaOH (270 kg) at 5-10° C. The phases were separated and the aqueous phase was washed with DCM (560 kg×2) to remove (S)-1-phenethylamine. A sample was taken for IPC (without any (S)-1-phenethylamine remaining). The aqueous phase was adjusted to pH 2.8-3.0 with 1 M aq. HCl (300 kg) at 5-10° C. The aqueous phase was extracted with DCM (560 kg×3). The DCM phases were combined and washed with water (421 kg, 5.0 vol.). The DCM phase was dried over Na₂SO₄ (84 kg, 1.0 w). After filtration the filter cake was rinsed with DCM (168 kg, 2.0 vol.). The filtrate was concentrated under reduced pressure at 30-40° C. A total of 1176.1 kg of a DCM solution of free acid Compound 9c (equivalent to 83.5 kg of neat Compound 9c based on HPLC assay) was obtained with 97.4% HPLC purity and 7.1 wt % assay in a 91.6% assay-corrected yield (Table 18).

TABLE 18a
HPLC Method for Compound 9c from part 2a

| Instrument: | HPLC (e.g., Agilent 1260 series) | |
|---|---|---|
| Column: | Agilent Poroshell 120 EC-C18 (4.6 × 100 mm, 2.7 μm) | |
| Mobile Phase: | A: 0.1% phosphoric acid in water | |
| | B: Acetonitrile | |
| Gradient: | Time (min) | B (%) |
| | 0.00 | 5 |
| | 20.0 | 50 |
| | 25.0 | 90 |
| | 32.0 | 90 |

| | Post time: 3 min |
|---|---|
| Flow Rate | 1.0 mL/min |
| UV Detector Wavelength: | 210 nm |
| Column Temperature: | 30° C. |
| Retention Times: | methyl (S)-3-(4-bromothiazol-2-yl)-2-((tert- |
| | butoxycarbonyl)amino) propanoate ≈ 20.1 min; |
| | (S)-3-(4-bromothiazol-2-yl)-2-((tert- |
| | butoxycarbonyl)amino)propanoic acid ≈ 16.3 min |

MS (ESI+):

Calculated for C₆H₁₄N₂O₂ (M+H⁺): 350.99

Found: 350.80

¹H NMR (400 MHz, CDCl₃):

δ 9.35 (s, 1H), 7.16 (s, 1H), 5.63 (d, J=6.3 Hz, 1H), 4.68 (d, J=5.0 Hz, 1H), 3.58 (d, J=4.7 Hz, 2H), 1.44 (s, 9H).

Part 2b—Alternative Preparation of Compound 9c: (S)-3-(4-bromothiazol-2-yl)-2-((tert-butoxycarbonyl)amino)propanoic acid

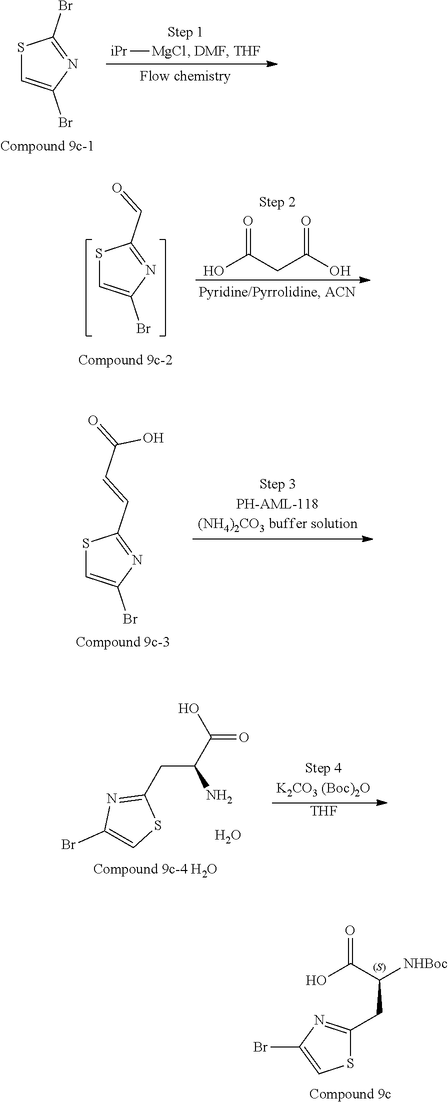

### Steps 1 and 2: Preparation of Compound 9c

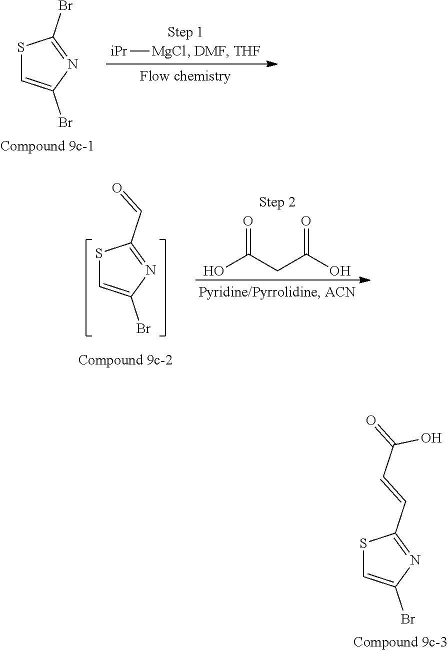

The following three solutions were prepared:
i) Solution 1: the reactor was charged with THE (10 L, 5.0 vol.) and Compound 9c-1 (2.0 kg, 8.2 mol, 1.0 eq), producing a clear solution;
ii) Solution 2: 2M i-PrMgCl in THE (14.4 L, 7.2 mol, 0.875 eq.); and
iii) Solution 3: DMF (1.2 kg, 16.5 mol, 2.0 eq.).

The flow rate of Pump 1 was adjusted to 44.1 mL/min for solution 1, the flow rate of Pump 2 was adjusted to 15.9 mL/min for solution 2, and the flow rate of Pump 3 was adjusted to 5.4 mL/min for solution 3. After heating the oil bath to 20° C., Pumps 1 and 2 were started at the same time, followed by Pump 3. After 2 min, the reaction mixture was collected. After 5 min, the reaction was monitored by IPC (HPLC purity: 3.6 A % of Compound 9c-1; 92.9 A % of Compound 9c-2). DCM (30 L, 15 vol.), followed by hydrochloric acid (628.5 g, 17.2 mol, 2.1 eq.) in water (10 L, 5.0 vol.) were charged into the reactor. The reaction mixture was then charged into 1.5 M HCl solution (12 L, 6 vol.) under 15±5° C. The organic phase was subsequently collected and washed with water (20 L×3). The organic phase was concentrated under reduced pressure at NMT 35° C. until the residual volume reached 6 L (3.0 vol.), then the solvent was swapped with MeCN (6.0 L×2). A total of 4.95 kg of Compound 9c-2 solution in MeCN (26 wt %) was obtained. To a reactor was charged Compound 9c-2 (4.95 kg of MeCN solution, 26 wt %, 6.7 mol, 1.0 eq.) and MeCN (6.5 L 0.5.0 vol.). Malonic acid (766.5 g, 1.1 eq.) and pyridine (2.1 kg, 4.0 eq.) were added into the reactor at 15-25° C., followed by pyrrolidine (95 g, 0.2 eq.). The mixture was stirred at 80±5° C. for 10 h. After confirming reaction completion, the reaction mixture was cooled to 5±5° C. DCM (650 mL, 0.5 vol.) was added into the mixture. Diluted HCl (980 g HCl in water (32.5 L, 25.0 vol.)) was added into the mixture dropwise until the pH was adjusted to about 2 at 5±5° C. The mixture was stirred at 5±5° C. for 2 h. The mixture was filtered, and the cake was rinsed with water (2.6 L, 2.0 vol.). The cake was rinsed with DCM (650 mL, 0.5 vol.). The wet cake was dried at 50-55° C. under reduced pressure for 12 h. A total of 1.45 kg of Compound 9c-3 was obtained as a solid with 99.7% HPLC purity as an off-white solid (Table 18b).

¹H NMR (400 MHz, DMSO-d6): δ 12.92 (s, 1H), 8.03 (s, 1H), 7.67 (d, 1H), 6.70 (d, 1H).

TABLE 18b
HPLC Method for Compound 9c from part 2b

| Instrument: | HPLC (e.g., Agilent 1260 series) | |
|---|---|---|
| Column: | Waters XBridge ® Shield RP18, | |
| | 4.6 × 150 mm, 3.5 um | |
| Mobile Phase: | A: 0.1% NH3H2O in water | |
| | B: Acetonitrile | |
| Gradient: | Time (min) | B (%) |
| | 0.00 | 5 |
| | 3.00 | 20 |
| | 8.00 | 27.5 |
| | 10.00 | 95 |
| | 21.00 | 95 |
| | 21.5 | 5Post time: 3 min |

| | |
|---|---|
| Flow Rate | 1.0 mL/min |
| UV Detector Wavelength: | 254 nm |
| Column Temperature: | 40° C. |
| Retention Times: | Compound 9c-2 ≈ 6.5 min |
| | Compound 9c-1 ≈ 11.9 min |

Step 3: Synthesis of Compound 9c-4·H₂O

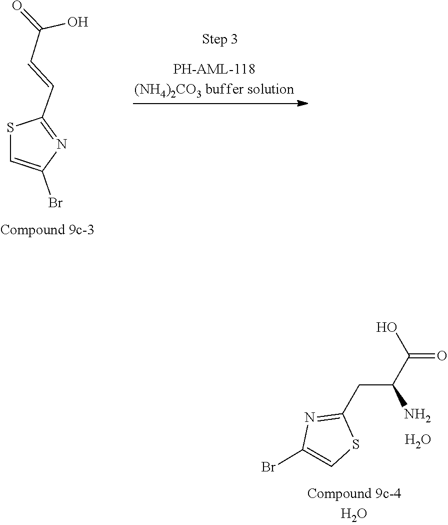

A reactor was charged with (NH₄)₂CO₃ buffer solution (600 mL, 10.0 vol., pH 9.8) under stirring at 34±2° C. The enzyme PH-AML-118 (600 mg, 1.0 wt %) and Compound 9c-3 (60 g, 257.6 mmol, 1.0 eq.) were added into the reactor. The mixture was stirred at 34±2° C. for 10 h and the reaction was monitored by IPC (HPLC purity: 96.3 A % Compound 9c-4 and 2.4 A % of Compound 9c-3, ee: 97.4%). The mixture was cooled to 25±5° C. and the temperature was maintained for 10 min. The mixture was then adjusted to a pH of 1 0.0±0.2 with 12 M HCl (382 mL, 4.7 vol.). The mixture was filtered, and the cake was rinsed with water (30 mL, 0.5 vol.). The filtrate was collected and was added into a reactor. The pH of the filtrate was adjusted to 1.8±0.1 with 50% aq. NaOH. Crystal seeds of Compound 9c-4 were subsequently added to the mixture, and the mixture was stirred for 2 h. The pH of the mixture was adjusted to 5.0±0.5 with 50% aq. NaOH (6 mL, 0.2 vol.). The mixture was heated to 50±5° C. and stirred for 2 h and was subsequently cooled to 40±5° C. and stirred for 30 min. The mixture temperature was incrementally reduced by 10±5° C. and stirred for 30 min for four times until a temperature of 0±5° C. was achieved. The mixture was subsequently stirred for 5 h at 0±5° C. The mixture was filtered, and the cake was rinsed with water (60 mL, 1.0 vol.). The wake cake was dried at 45±5° C. under reduced pressure for 16 h. A total of 57.5 g of Compound 9c-4 H₂O was obtained as a solid with 99.9% HPLC purity, ee≥99.9% (Table 18c).

TABLE 18c
HPLC Method for Compound 9c-4

| Instrument: | HPLC (e.g., Agilent 1260 series) | |
|---|---|---|
| Column: | InfinityLab Poroshell 120 chiral-T, | |
| | 4.6*150 mm, 2.7 um | |
| Mobile Phase: | A: 0.1% FA in water | |
| | B: MeOH | |
| Gradient: | Time (min) | B (%) |
| | 0.00 | 5 |
| | 10.00 | 80 |
| | 15.00 | 95 |
| | 20.00 | 95 |
| | 21.10 | 5 |
| | 25.00 | 5 |

| | |
|---|---|
| Flow Rate | 0.5 mL/min |
| UV Detector Wavelength: | 254 nm |
| Column Temperature: | 40° C. |
| Retention Times: | Compound 9c-4 ≈ 6.7 min |
| | Isomer ≈ 7.4 min |
| | Compound 9c-3 ≈ 10.0 min |

¹H NMR (400 MHz, D₂O): δ 7.49 (s, 1H), 4.13 (dd, 1H), 3.58 (dd, 1H).

### Step 4: Synthesis of Compound 9c

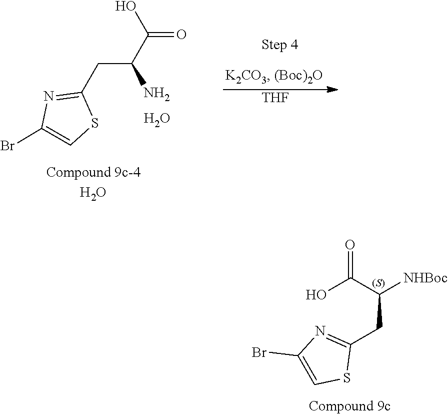

A reactor was charged with K₂CO₃ (14.4 g, 104 mmol, 1.4 eq.) and water (60 mL, 3.0 vol.). The mixture was adjusted to 20±5° C. Compound 9c-4 H₂O (20.0 g, 74.3 mmol, 1.0 eq.) was added into the reactor. The mixture was heated to 45+5° C., and was stirred to a clear solution. The solution of (Boc)₂O (17.8 g, 81.7 mmol, 1.1 eq. in 20 mL THF) was added into the reactor. The mixture was stirred at 45±5° C. for 1 h, and the reaction was monitored by IPC (HPLC purity: 98.8 A % of Compound 9c, Compound 9c-4 was not detected). The mixture was cooled to 20±5° C. DCM (40 mL, 2.0 vol.) was added into the reactor. The mixture was adjusted to a pH of 2-3 with 3M HCl, and was stirred for 30 min. The mixture was separated and the organic phase was collected. The aqueous phase was extracted with DCM (40 mL, 2.0 vol.), and the organic phase was combined. DCM (100 mL, 5.0 vol.) was added into the organic phase, and the mixture was concentrated under reduced pressure at NMT 40° C. until 4-5 vol. DCM (100 mL, 5.0 vol.) was added into the residual. The mixture was concentrated under reduced pressure at NMT 40° C. until 4-5 vol was obtained. DCM (100 mL, 5.0 vol.) was added into the residual mixture, and 180 g of a DCM solution of Compound 9c was obtained as the free acid with 99.92% HPLC purity and 14 wt % in a 90% assay-corrected yield.

¹H NMR (400 MHz, CDCl₃): δ 9.35 (s, 1H), 7.16 (s, 1H), 5.63 (d, 1H), 4.68 (d, 1H), 3.58 (d, 2H), 1.44 (s, 9H).

Part 3—Preparation of Compound 9d methyl (S)-hexahydropyridazine-3-carboxylate dihydrochloride

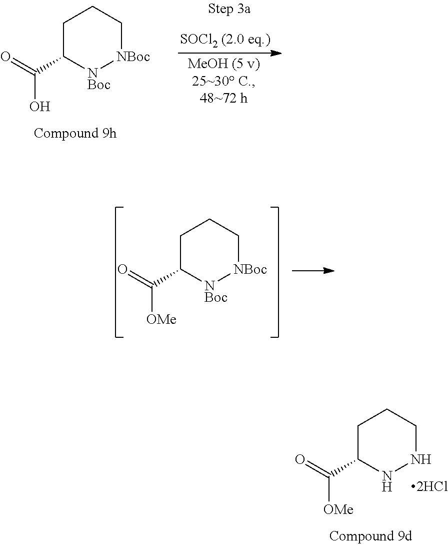

To a reactor was charged MeOH (371 kg, 5.0 vol.) and (S)-1,2-bis(tert-butoxycarbonyl) hexahydropyridazine-3-carboxylic acid (Compound 9h) (93.9 kg, 284.2 mol, 1.0 eq.). SOCl₂ (67.6 kg, 568.4 mol, 2.0 eq.) was added dropwise into the mixture at 10-20° C. The reaction mixture was warmed to 35-40° C. and stirred for 43 h. A sample was taken for IPC (HPLC purity showed: 98.5 A % of Compound 9d and 0 A % Compound 9h). The reaction mixture was concentrated to 2 vol. (ca. 190 L) under reduce pressure at 35-40° C. Dioxane (193 kg, 2 vol.) was added into the mixture and concentrated to 2 vol. (ca. 190 L) under reduce pressure at 35-40° C. Dioxane (193 kg, 2 vol.) was added into the mixture and concentrated to 2 vol. (ca. 190 L) under reduce pressure at 35-40° C. (OT). Dioxane (193 kg, 2 vol.) was added into the mixture and concentrated to 2 vol. (ca. 190 L) under reduced pressure at 35-40° C. The resulting mixture was diluted with DCM (250 kg, 2 vol.). 598 Kg of a dioxane/DCM solution of Compound 9d was obtained with 95.7% HPLC purity and 10.3 wt % assay by HPLC with quantitative yield (Table 19).

TABLE 19
HPLC Method for Compound 9d

| Instrument: | HPLC (e.g., Agilent 1260 series) | |
|---|---|---|
| Column: | Agilent Eclipse Plus C18 | |
| | (4.6 × 100 mm, 3.5 μm) | |
| Mobile Phase: | A: 10 mM (NH₄)₂HPO₄ in water; | |
| | B: Acetonitrile | |
| Gradient: | Time(min) | % B |
| | 0.0 | 2 |
| | 2.0 | 2 |
| | 10.0 | 25 |
| | 15.0 | 85 |
| | 20.0 | 85 |

| | Post time: 5.0 min |
|---|---|
| Flow Rate | 0.8 mL/min |
| UV Detector Wavelength: | 210 nm |
| Column Temperature: | 30° C. |
| Retention Times: | methyl (S)-hexahydropyridazine- |
| | 3-carboxylate |
| | dihydrochloride ≈ 6.9 min; |
| | (S)-1,2-bis(tert- |
| | butoxycarbonyl)hexahydropyridazine-3- |
| | carboxylic acid ≈ 13.1 min |

MS (ESI+):

Calculated for C₆H₁₄N₂₀₂ (M+H⁺): 145.09

Found: 145.10

¹H NMR (400 MHz, DMSO-d₆):

δ 3.96 (dd, J=10.5, 2.5 Hz, 1H), 3.63 (s, 3H), 3.06 (s, 1H), 2.91 (dd, J=16.2, 7.4 Hz, 1H), 1.89 (d, J=10.6 Hz, 2H), 1.78 (dd, J=9.8, 3.4 Hz, 1H), 1.65-1.47 (m, 1H).

Part 4—Preparation of Compound 9e methyl (S)-1-((S)-3-(4-bromothiazol-2-yl)-2-((tert-butoxycarbonyl)amino)propanoyl)hexahydropyridazine-3-carboxylate

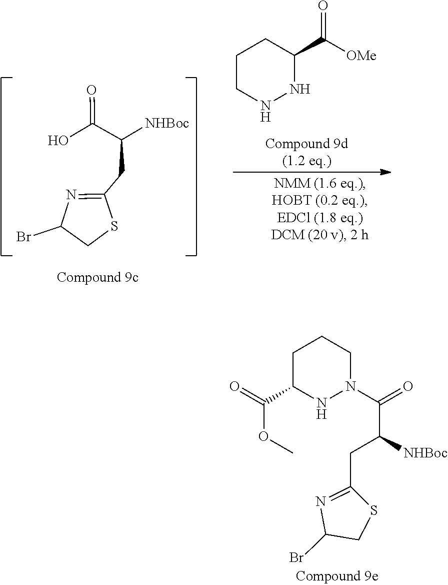

To a reactor was charged Compound 9d (598.0 kg of dioxane/DCM solution, 10.3 wt %, 284.1 mol, 1.2 eq.) and DCM (553 kg, 5.0 vol.). The reactor was evacuated and backfilled with nitrogen 3 times. The mixture was cooled to 0-5° C. NMM (38.3 kg, 378.8 mol, 1.6 eq.) was added dropwise into the mixture at 0-5° C. and stirred for another 30 min. Compound 9c (1176 kg of DCM solution, 7.1 wt %, 236.7 mol, 1.0 eq.) was added dropwise into the mixture at 0-5° C. and stirred for another 30 min. HOBt (0.64 kg, 4.7 mol, 0.02 eq.) and EDCI (81.7 kg, 426.1 mol, 1.8 eq.) were added into the mixture at 0-5° C. and stirred for another 1 h. A sample taken for IPC (HPLC purity: 86.5 A % of Compound 9e with no Compound 9c remaining). The reaction mixture was washed with water (831 kg×4, 10 vol.×4). The DCM phase was concentrated under reduced pressure at 25-30° C. to 2 vol. (ca. 200 L). MTBE (307 kg, 5 vol.) was added into the above DCM solution. The organic phase was concentrated under reduced pressure at 25-30° C. to 2 vol. (ca. 200 L). MTBE (307 kg, 5 vol.) was added into the above solution. The mixture was concentrated under reduced pressure at 25-30° C. to 2 vol. (ca. 200 L). MTBE (307 kg, 5 vol.) was added into the above solution. The mixture was concentrated under reduced pressure at 25-30° C. to 2 vol. (ca. 200 L). MTBE (184 kg, 3 vol.) was added into the above solution. n-Heptane (141 kg, 2.5 vol.) was added dropwise into the above solution at 25-30° C. The resulting mixture was stirred at 15-20° C. for 30 min. The resulting mixture was cooled to 0-10° C. and stirred for another 60 min. The resulting slurry was filtered and the filter cake rinsed with 1:1 (vol./vol.) n-heptane/MTBE (141 kg, 2 vol.). The filter cake was dried at 35-40° C. under reduced pressure. A total of 105.5 kg of Compound 9e was obtained as a white solid with 99.6 A % HPLC purity and 99.4 wt % HPLC assay in an 92.8% assay-corrected yield (Table 20).

TABLE 20
HPLC Method for Compound 9e

| Instrument: | HPLC (e.g., Agilent 1260 series) | |
|---|---|---|
| Column: | Agilent Poroshell 120 EC-C18 (4.6 × 100 mm, 2.7 μm) | |
| Mobile Phase: | A: 0.1% phosphoric acid in water | |
| | B: Acetonitrile | |
| Gradient: | Time (min) | B (%) |
| | 0.00 | 5 |
| | 7.00 | 90 |
| | 12.0 | 90 |

| | Post time: 3 min |
|---|---|
| Flow Rate | 1.0 mL/min |
| UV Detector Wavelength: | 210 nm |
| Column Temperature: | 30° C. |
| Retention Times: | (S)-3-(4-bromothiazol-2-yl)-2-((tert- |
| | butoxycarbonyl)amino)propanoic acid ≈ 3.9 min; |
| | methyl (S)-1-((S)-3-(4-bromothiazol-2-yl)-2-((tert- |
| | butoxycarbonyl)amino)propanoyl)hexahydropyridazine-3- |
| | carboxylate ≈ 5.4 min |

MS (ESI+):

Calculated for C₁₇H₂₅BrN₄O₅S (M+H⁺): 477.07

Found: 477.20

¹H NMR (400 MHz, CD₃₀D-d₄):

δ 7.44 (s, 1H), 5.64-5.31 (m, 1H), 3.92 (s, 1H), 3.74 (s, 3H), 3.61 (d, J=3.9 Hz, 1H), 3.39 (dd, J=14.5, 5.0 Hz, 1H), 3.29-3.18 (m, 2H), 1.99 (dd, J=8.4, 5.0 Hz, 1H), 1.86-1.62 (m, 3H), 1.40 (d, J=18.6 Hz, 9H).

Part 5—Preparation of Compound 9f (methyl (S)-1-((S)-2-amino-3-(4-bromothiazol-2-yl) propanoyl)hexahydropyridazine-3-carboxylate)

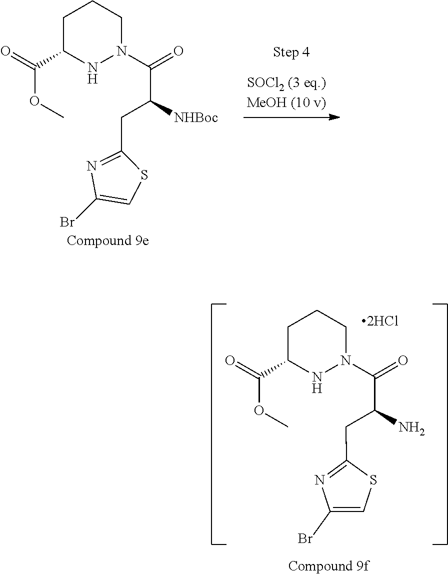

To a reactor was charged MeOH (960 kg, 10 vol.) and methyl (S)-1-((S)-3-(4-bromothiazol-2-yl)-2-((tert-butoxycarbonyl)amino)propanoyl)hexahydropyridazine-3-carboxylate (Compound 9e) (121.5 kg, 254.5 mol, 1.0 eq.). The reactor was evacuated and backfilled with nitrogen 3 times. SOCl₂ (90.8 kg, 763.6 mol, 3.0 eq.) was added dropwise into the mixture at 0-10° C. The resulting mixture was heated to 30-40° C. and stirred at this temperature for 2 h. A sample was taken for IPC (HPLC: 98.2 A % of Compound 9f and 0 A % of Compound 9e). The reaction mixture was concentrated at 30-40° C. to 150-250 L and diluted with DCM (808 kg, 5 vol.). The mixture was adjusted pH to 10.0-10.4 with 15 wt % aq. Na₂CO₃ (2673 kg, 22 wt) at 0-10° C. After phases separation, the aqueous phase was extracted with DCM (808 kg×2, 2×5 vol.). The DCM phases were combined and washed with 26% aq. NaCl (3×1215 kg, 3×10 vol.). The DCM phase was concentrated under reduced pressure at 30-40° C. to 1200-1500 L. A DCM solution of Compound 9f (1612.3 kg, 5.35 wt %) was obtained with 99.3 A % purity in a 91.1% corrected yield (Table 21).

TABLE 21
HPLC method for Compound 9f

| Instrument: | HPLC (e.g., Agilent 1260 series) | |
|---|---|---|
| Column: | Agilent Poroshell 120 EC-C18 (4.6 × 100 mm, 2.7 μm) | |
| | PN: 696975-902 | |
| Mobile Phase: | A: 0.1% phosphoric acid in water | |
| | B: Acetonitrile | |
| Gradient: | Time (min) | B (%) |
| | 0.0 | 5 |
| | 2.0 | 20 |
| | 10.0 | 35 |
| | 15.0 | 90 |
| | 20.0 | 90 |
| | 20.1 | 5 |
| | 25.0 | 5 |

| | |
|---|---|
| Flow Rate | 1.0 mL/min |
| UV Detector Wavelength: | 210 nm |
| Column Temperature: | 30° C. |
| Diluent: | Acetonitrile |
| Injection Volume: | 5 μL |
| Retention Times: | methyl (S)-1-((S)-3-(4-bromothiazol-2-yl)-2-((tert- |
| | butoxycarbonyl)amino)propanoyl)hexahydropyridazine-3- |
| | carboxylate ≈ 14.2 min; |
| | methyl (S)-1-((S)-2-amino-3-(4-bromothiazol-2- |
| | yl)propanoyl)hexahydropyridazine-3-carboxylate ≈ 4.9 min |

LCMS (ESI+)

Calculated for C₁₂H₁₇BrN₄O₃S (M+H⁺): 377.02

Found: 377.10

¹H NMR (400 MHz, CD₃OD)

δ 7.57 (s, 1H), 5.45 (br, 1H), 3.80 (br, 1H), 3.77 (s, 3H), 3.67 (dd, J=16.1, 4.3 Hz, 2H), 3.51 (dd, J=16.1, 8.0 Hz, 2H), 1.91-2.09 (m, 2H), 1.83-1.67 (m, 2H).

Part 6—Preparation of Compound 9g—methyl (S)-1-((S)-3-(4-bromothiazol-2-yl)-2-((1S,2S)-2-methylcyclopropane-1-carboxamido)propanoyl)hexahydropyridazine-3-carboxylate

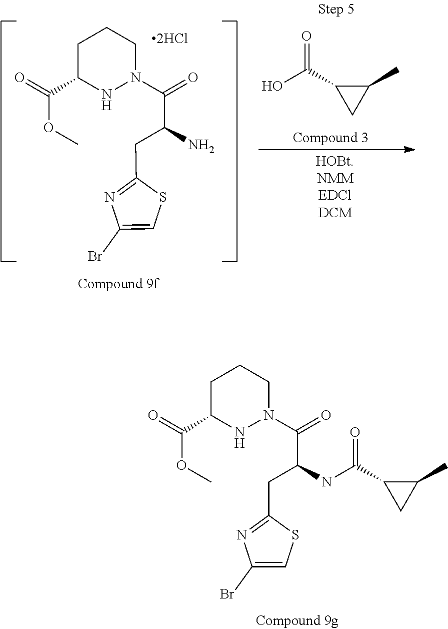

Methyl (S)-1-((S)-2-amino-3-(4-bromothiazol-2-yl)propanoyl)hexahydropyridazine-3-carboxylate (Compound 9f) (1612.3 kg of a DCM solution, 5.4 wt % assay by HPLC, 230.8 mol, 1.0 eq.) and 212.4 kg DCM were charged into a reactor. The reactor was evacuated and backfilled with nitrogen 3 times. NMM (37.3 kg, 369.2 mol, 1.6 eq.) was added dropwise into the mixture at 0-10° C. and stirred at this temperature for 10 min. Compound 3 (30.0 kg, 300.0 mol, 1.3 eq.) was added dropwise into the mixture at 0-10° C. and stirred at this temperature for 10 min. HOBt (0.62 kg, 4.6 mol, 0.02 eq.) and EDCI (79.6 kg, 415.4 mol, 1.8 eq.) were added into the mixture at 0-10° C. The mixture was stirred at 0-10° C. for 1-3 h. A sample was taken for IPC (HPLC: 92.5 A % of Compound 9g and 0 A % of Compound 9f). The DCM phase was washed three times with water (871 kg×3, 10 vol.×3). The DCM phase was concentrated under reduced pressure 30-40° C. to 2-3 vol. (180-270 L). n-heptane (355 kg, 6 vol.) was added dropwise into the above solution at 20-40° C. The resulting mixture was stirred at 10-20° C. for 30 min and stirred at 0-5° C. for 5 h. The resulting mixture was filtered and the filter cake was rinsed with 2:1 (vol/vol.) n-heptane/DCM (174 kg, 2 vol.). The filter cake was dried at 30-40° C. under reduced pressure for 12 h. A total of 94.8 kg of Compound 9g was obtained as a white solid with 99.8 A % HPLC purity and 99.5 wt % assay in an 89% assay-corrected yield (Table 22).

TABLE 22
HPLC method for Compound 9g

| Instrument: | HPLC with UV/DAD detector | |
|---|---|---|
| Column: | Agilent Poroshell 120 EC-C18 | |
| | (2.7 μm, 4.6 × 100 mm) | |
| | PN: 695975-902 | |
| Mobile Phase: | A: 0.1% H₃PO₄ in Water | |
| | B: MeCN | |
| Gradient: | Time (min) | B (%) |
| | 0.0 | 5 |
| | 4.0 | 35 |
| | 12.0 | 90 |
| | 15.0 | 90 |
| | 15.1 | 5 |
| | 20.0 | 5 |

| | |
|---|---|
| Flow Rate | 1.0 mL/min |
| UV Detector | 210 nm |
| Wavelength: | |
| Run time | 20.00 min |
| Column Temperature: | 30° C. |
| Diluent: | MeCN |
| Needle Wash | MeCN |
| Injection Volume: | 2 μL |
| Sample Tray | Room temperature |
| Temperature | |
| Retention Times: | (S)-1-((S)-2-amino-3-(4-bromothiazol-2- |
| | yl)propanoyl)hexahydropyridazine-3- |
| | carboxylate ≈ 4.8 min; DCM ≈ 5.7 min; |
| | methyl (S)-1-((S)-3-(4-bromothiazol-2-yl)- |
| | 2-((1S,2S)-2-methylcyclopropane-1- |
| | carboxamido)propanoyl)hexahydropyridazine- |
| | 3-carboxylate ≈ 7.7 min |

LCMS (ESI+):

Calculated for C₁₇H₂₃BrN₄O₄S (M+H⁺): 459.06

Found: 459.30

¹H NMR (400 MHz, DMSO-d₆):

δ 8.15 (d, J=8.5 Hz, 1H), 7.69 (s, 1H), 5.59 (td, J=8.0, 5.3 Hz, 1H), 5.32 (d, J=9.7 Hz, 1H), 3.93-3.76 (m, 1H), 3.66 (s, 3H), 3.50 (dd, J=15.9, 7.8 Hz, 1H), 3.33-3.24 (m, 1H), 3.23-2.92 (m, 2H), 1.95-1.76 (m, 1H), 1.78-1.39 (m, 4H), 1.17-0.93 (m, 4H), 0.83 (dt, J=7.8, 3.9 Hz, 1H), 0.47 (dt, J=8.1, 4.4 Hz, 1H).

Part 7—Preparation of Compound 9—(S)-1-((S)-3-(4-bromothiazol-2-yl)-2-((1S,2S)-2-methylcyclopropane-1-carboxamido)propanoyl)hexahydropyridazine-3-carboxylic acid

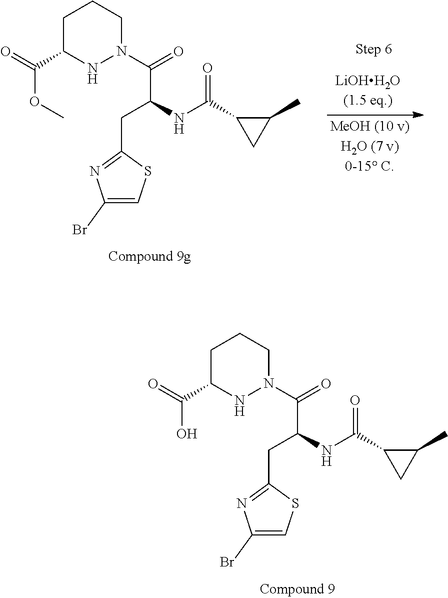 [SMILES: COC(=O)[C@@H]1CCCN(C(=O)[C@H](Cc2nc(Br)cs2)NC(=O)[C@H]2C[C@@H]2C)N1 ; C[C@H]1C[C@@H]1C(=O)N[C@@H](Cc1nc(Br)cs1)C(=O)N1CCC[C@@H](C(=O)O)N1]

(Methyl(S)-1-((S)-3-(4-bromothiazol-2-yl)-2-((1S,2S)-2-methylcyclopropane-1-carboxamido)propanoyl)Hexahydropyridazine-3-carboxylate) (Compound 9g) (94.0 kg, 204.6 mol, 1.0 eq.) and MeOH (743 kg, 10 vol.) were charged into a reactor. Aq. LiOH solution was added dropwise into the mixture at 2-4° C. The mixture was stirred at 0-5° C. for 7 h. A sample was taken for IPC 1 (HPLC: 99.4 A % of Compound 9 and 0.2 A % of Compound 9g). The solution was filtered through a filter tank and an online precision filter. Took a sample for IPC 2 (HPLC: 99.4 A % of Compound 9 and 0.2 A % of Compound 9g). The reaction mixture was adjusted to pH 6.2-7.2 with 1 M aq. HCl at 0-5° C. and followed by concentration under reduced pressure at 25-35° C. (OT) until the residual volume reached 650-750 L. EtOAc (846 kg, 10 vol.) was charged into the reactor. The reaction mixture was adjusted to pH 2.8-3.2 with 1 M aq. HCl at 0-5° C. Separate the layers. The aqueous phase was extracted with EtOAc two times (846 kg, 10 vol. and 423 kg, 5 vol.). The EtOAc phases were combined and washed with 26 wt % aq. NaCl (1050 kg, 10 vol.). The EtOAc phases was filtered with a filter tank. The EtOAc phase was concentrated under reduced pressure at 35-45° C. (OT) to 380-470 L. Seeds crystals (45 g) were added to the mixture and stirred at 5-15° C. for 10 min. n-Heptane (320 kg, 5 vol.) was added into the above EtOAc solution. The resulting mixture was concentrated under reduced pressure at 35-45° C. to 380-470 L. n-Heptane (320 kg, 5 vol.) was added into the above EtOAc solution. The resulting mixture was concentrated under reduced pressure at 35-45° C. to 380-470 L. n-Heptane (320 kg, 5 vol.) was added into the above EtOAc solution. The resulting mixture was concentrated under reduced pressure at 35-45° C. to 380-470 L. The resulting mixture was stirred at 10-20° C. for 0.5-1.5 h and stirred at 1-5° C. for 2-4 h. The mixture was filtered and the filter cake was rinsed with n-heptane (128 kg, 2 vol.). The filter cake was dried at 35-45° C. for 10 h under reduced pressure. A total of 87.7 kg of Compound 9 was obtained as a white solid with 99.2% HPLC purity and 97.9 wt % assay by HPLC in a 94.2% assay-corrected yield (Table 23).

TABLE 23
HPLC method for Compound 9

| Instrument: | HPLC (e.g. Agilent 1260 series) | |
|---|---|---|
| Column: | Agilent Poroshell 120 EC-C18 | |
| | (2.7 μm, 4.6 × 100 mm); | |
| | PN: 695975-902 | |
| Mobile Phase: | A: 0.1% H₃PO₄ in Water | |
| | B: MeCN | |
| Gradient: | Time (min) | B (%) |
| | 0.0 | 5 |
| | 15.0 | 90 |
| | 17.0 | 90 |
| | 17.1 | 5 |
| | 20.0 | 5 |

| | |
|---|---|
| Flow Rate | 1.0 mL/min |
| UV Detector | 210 nm |
| Wavelength: | |
| Run time | 20.00 min |
| Column Temperature: | 30° C. |
| Diluent: | MeCN:H₂O = 1:1 (v/v) |
| Needle Wash | MeCN:H₂O = 1:1 (v/v) |
| Injection Volume: | 5 μL |
| Sample Tray | Room temperature |
| Temperature | |
| Retention Times: | (S)-1-((S)-3-(4-bromothiazol-2-yl)- |
| | 2-((1S,2S)-2-methylcyclopropane-1- |
| | carboxamido)propanoyl)hexahydropyridazine- |
| | 3-carboxylic acid ≈ 7.6 min; RRT ≈ 1.00; |
| | Methyl(S)-1-((S)-3-(4-bromothiazol-2- |
| | yl)-2-((1S,2S)-2-methylcyclopropane- |
| | 1-carboxamido)propanoyl) |
| | Hexahydropyridazine-3- |
| | carboxylate ≈ 9.0 min; RRT ≈ 1.18 |

LCMS (ESI+):

Calculated for C₁₆H₂₁BrN₄O₄S (M+H⁺): 445.05

Found: 445.20

¹H NMR (400 MHz, CD₃₀D)

δ 7.39 (d, J=5.3 Hz, 1H), 5.70 (dd, J=6.9, 5.8 Hz, 1H), 4.05 (d, J=7.3 Hz, 1H), 3.46 (dd, J=9.5, 3.6 Hz, 1H), 3.39 (dd, J=14.6, 5.6 Hz, 1H), 3.27 (dt, J=3.3, 2.0 Hz, 1H), 2.99 (s, 1H), 2.06-1.91 (m, 1H), 1.79 (td, J=9.0, 4.2 Hz, 1H), 1.73-1.60 (m, 2H), 1.36 (dt, J=8.3, 4.3 Hz, 1H), 1.18 (dtd, J=10.0, 6.1, 4.0 Hz, 1H), 1.04 (d, J=6.0 Hz, 3H), 0.97 (dt, J=8.6, 4.2 Hz, 1H), 0.55 (ddd, J=8.1, 6.2, 3.9 Hz, 1H).

### Example 7. Synthetic procedure for Compound A—(1S,2S)—N-[(7S,13S)-21-ethyl-20-{2-[(1S)-1-methoxyethyl]-5-(4-methylpiperazin-1-yl)pyridin-3-yl}-17,17-dimethyl-8,14-dioxo-15-oxa-4-thia-9,21,27,28-tetraazapentacyclo[17.5.2.1{2,5}0.1 {9,13}0.0{22,26}]octacosa-1(25),2,5(28),19,22(26),23-hexaen-7-yl]-2-methylcyclopropane-1-carboxamide

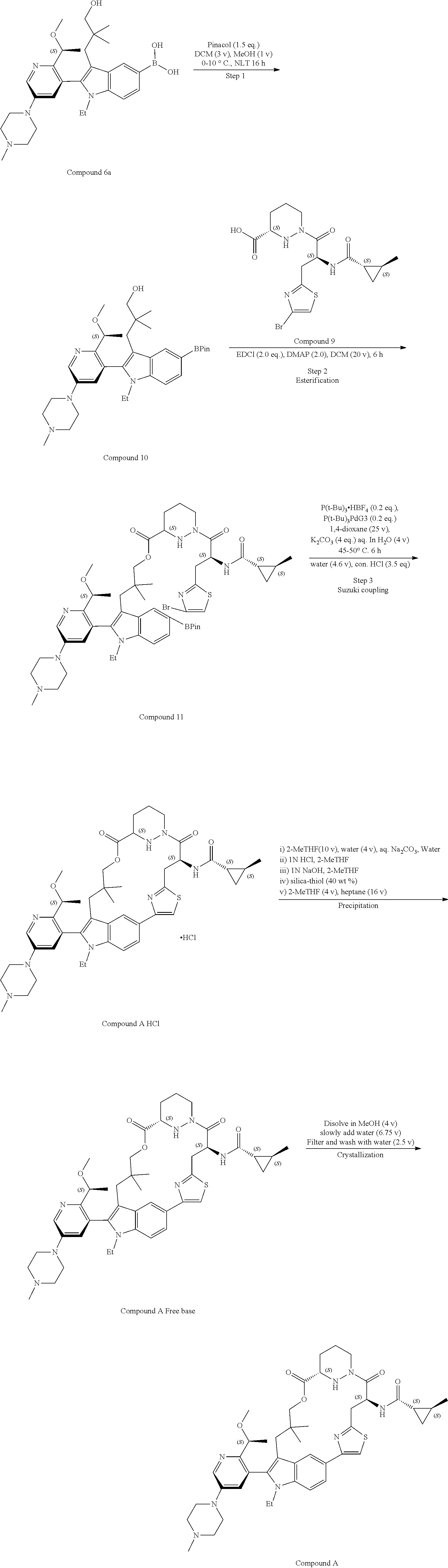

Part 1—Synthesis of Compound 10 (3-(1-ethyl-2-{2-[(1S)-1-methoxyethyl]-5-(4-methylpiperazin-1-yl)pyridin-3-yl}-5-(tetramethyl-1,3,2-dioxaborolan-2-yl)-1H-indol-3-yl)-2,2-dimethylpropan-1-ol)

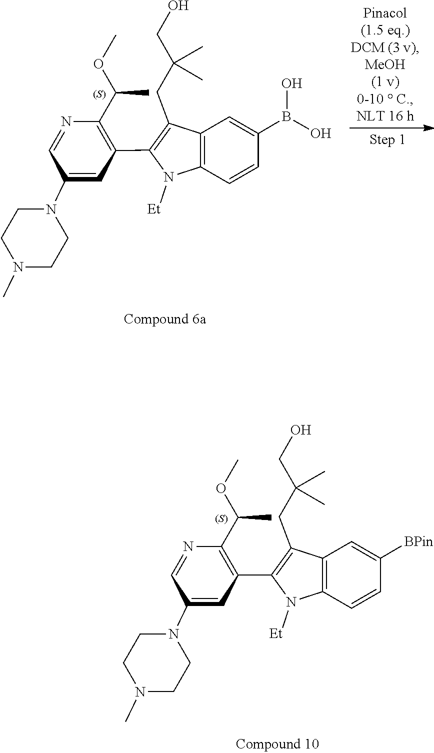

To a reactor was charged DCM (36.25 kg), MeOH (7.05 kg), [1-ethyl-3-(3-hydroxy-2,2-dimethylpropyl)-2-{2-[(1S)-1-methoxyethyl]-5-(4-methylpiperazin-1-yl)pyridin-3-yl}-1H-indol-5-yl]boronic acid (Compound 6a) (8.90 kg, 17.50 mol, 1.0 equiv) and pinacol (3.12 kg, 26.40 mol, 1.5 equiv). The solution was stirred at 25° C. for 18 hours. A sample was taken and diluted with DMSO to run HPLC analysis (criterion: area % of Compound 6a≤3%, result: area % of Compound 6a=0.4%). The solution was concentrated under reduced pressure until the residue volume was around 11 L. DCM (36.10 kg) was charged and then the resultant solution was concentrated under reduced pressure until the residue volume was around 11 L. The same process was repeated for another 9 times to get acceptable residual MeOH. A sample was taken for GC analysis to check residue MeOH (criterion: content of MeOH s 100 ppm, result: content of MeOH=60 ppm). The resultant solution of Compound 10 in DCM was used in next step without further purification (weight of DCM solution: 30.74 kg, 95.2% a/a purity, content of Compound 10: 32.0% w/w assay, 16.66 mol, 95.2% yield, Table 24).

TABLE 24
HPLC Method for Part 1 of Example 7

| Column: | Xbridge C18, 4.6 mm × 150 mm, 3.5 μm | | |
|---|---|---|---|
| Mobile Phase: | A: 10 mM NH₄OAc in water | | |
| | B: MeCN | | |
| Gradient: | Time (min) | A % | B % |
| | 0.0 | 70 | 30 |
| | 12.0 | 20 | 80 |
| | 15.0 | 5 | 95 |
| | 15.1 | 70 | 30 |

| | |
|---|---|
| Flow Rate: | 1.0 mL/min |
| Post Time | 4.0 min |
| UV Detector | 210 nm |
| Wavelength: | |
| Column Temperature: | 40° C. |
| Retention Times: | [1-ethyl-3-(3-hydroxy-2,2-dimethylpropyl)-2-{2- |
| | [(1S)-1-methoxyethyl]-5-(4- |
| | methylpiperazin-1-yl)pyridin-3-yl}- |
| | 1H-indol-5-yl]boronic acid: 3.9 min; |
| | 3-(1-ethyl-2-{2-[(1S)-1-methoxyethyl]- |
| | 5-(4-methylpiperazin-1-yl)pyridin-3-yl}- |
| | 5-(tetramethyl-1,3,2-dioxaborolan-2-yl)-1H- |
| | indol-3-yl)-2,2-dimethylpropan-1-ol: 10.8 min |

LCMS (ESI+)

Calculated for C₃₄H₅₁BN₄O₄ (M+H⁺): 591.40

Found: 591.40

¹H NMR (400 MHz, CDCl₃)

δ 8.51 (d, J=2.9 Hz, 1H), 8.23 (s, 1H), 7.72 (dd, J=8.3, 1.0 Hz, 1H), 7.37 (d, J=8.2 Hz, 1H), 7.17 (d, J=2.9 Hz, 1H), 4.12-3.88 (m, 3H), 3.30 (dt, J=11.7, 3.9 Hz, 6H), 3.04 (s, 3H), 2.81 (d, J=14.1 Hz, 1H), 2.63 (t, J=5.0 Hz, 3H), 2.38 (s, 3H), 2.33 (d, J=14.1 Hz, 1H), 1.44 (d, J=6.2 Hz, 3H), 1.39 (s, 12H), 1.22 (t, J=7.1 Hz, 3H), 0.89 (t, J=6.8 Hz, 2H), 0.83 (d, J=7.3 Hz, 6H).

Part 2—Synthesis of Compound 11—2-[(1-ethyl-2-{2-[(1S)-1-methoxyethyl]-5-(4-methylpiperazin-1-yl)pyridin-3-yl}-5-(tetramethyl-1,3,2-dioxaborolan-2-yl)-1H-indol-3-yl)methyl]-2-methylpropyl (3S)-1-[(2S)-3-(4-bromo-1,3-thiazol-2-yl)-2-{[(1S,2S)-2-methylcyclopropyl]formamido}propanoyl]-1,2-diazinane-3-carboxylate

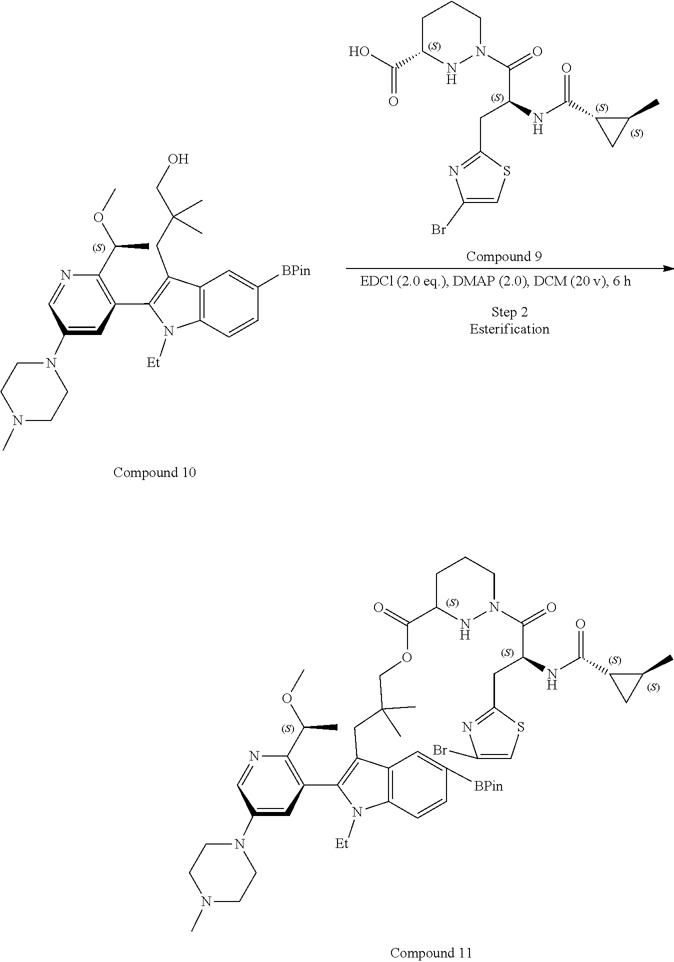

To a reactor was charged DCM solution of 3-(1-ethyl-2-{2-[(1S)-1-methoxyethyl]-5-(4-methylpiperazin-1-yl)pyridin-3-yl}-5-(tetramethyl-1,3,2-dioxaborolan-2-yl)-1 H-indol-3-yl)-2,2-dimethylpropan-1-ol (Compound 10) (30.56 kg, content of Compound 10: 32.0% w/w assay, 16.56 mol,1.0 equiv.), DCM (100.45 kg), (3S)-1-[(2S)-3-(4-bromo-1,3-thiazol-2-yl)-2-{[(1S,2S)-2-methylcyclopropyl]formamido}propanoyl]-1,2-diazinane-3-carboxylic acid (Compound 9) (8.52 kg, 19.13 mol, 1.16 equiv) and DMAP (3.05 kg, 2.50 mol, 1.5 equiv). The solution was cooled to 19° C. and EDCI (6.39 kg, 3.33 mol, 2.0 equiv) was added portion wise with stirring at 15˜20° C. The resulting solution was stirred for 14 hours at 15˜20° C. A sample was taken and diluted with MeCN to check IPC purity by HPLC analysis (criterion: area % of Compound 10 s 5%, actual result: area % of Compound 10: 3%). The reaction mixture was quenched with water, washed with aqueous HCl (0.2M, 171.5 kg), aqueous NaHCO₃ (8% w/w, 155.7 kg) and water (98.4 kg) subsequentially. Then the DCM phase was separated and concentrated under reduced pressure until the residue volume was around 25 L. MTBE (37.05 kg, 3V) was added and the resultant solution was concentrated under reduced pressure until the residue volume was around 25 L. The solvent exchange process was repeated for another 3 times. The resulting MTBE solution was added to pre-cooled n-heptane (25 L) at −10° C. dropwise. Then the resulting suspension was stirred for 12.5 hours at −10° C. The slurry was filtered, filter-cake was washed with n-heptane (6.8 kg). The wet-cake was dried at 30° C. under vacuum to afford Compound 11 as a white solid (17.29 kg, 86.5% a/a purity, 81.9% w/w assay, 13.91 mol, 84% yield, Table 25).

TABLE 25
HPLC Method for Part 2 of Example 7

| Column: | Eclipse plus C8, 4.6 mm × 150 mm, 3.5 μm | | |
|---|---|---|---|
| Mobile Phase: | A: 0.05% TFA in water | | |
| | B: 0.05% TFA in MeCN | | |
| Gradient: | Time (min) | A % | B % |
| | 0.0 | 60 | 40 |
| | 7.0 | 40 | 60 |
| | 9.0 | 5 | 95 |
| | 15.0 | 5 | 95 |

| | |
|---|---|
| Flow Rate: | 0.8 mL/min |
| Post Time | 4.0 min |
| UV Detector | 210 nm |
| Wavelength: | |
| Column Temperature: | 30° C. |
| Retention Times: | DMAP: 1.8 min; |
| | (3S)-1-[(2S)-3-(4-bromo-1,3- |
| | thiazol-2-yl)-2-{[(1S,2S)-2- |
| | methylcyclopropyl]formamido}propanoyl]- |
| | 1,2-diazinane-3-carboxylic acid: 3.6 min; |
| | 3-(1-ethyl-2-{2-[(1S)-1-methoxyethyl]- |
| | 5-(4-methylpiperazin-1-yl)pyridin-3-yl}-5- |
| | (tetramethyl-1,3,2-dioxaborolan-2-yl)-1H- |
| | indol-3-yl)-2,2-dimethylpropan-1-ol: 4.7 min; |
| | 2-[(1-ethyl-2-{2-[(1S)-1-methoxyethyl]- |
| | 5-(4-methylpiperazin-1-yl)pyridin-3-yl}-5- |
| | (tetramethyl-1,3,2-dioxaborolan-2-yl)-1H- |
| | indol-3-yl)methyl]-2-methylpropyl (3S)-1-[(2S)- |
| | 3-(4-bromo-1,3-thiazol-2-yl)-2-{[(1S,2S)-2- |
| | methylcyclopropyl]formamido}propanoyl]-1,2- |
| | diazinane-3-carboxylate: 9.4 min |

LCMS (ESI+)

Calculated for C₅₀H₇₀BBrN₈O₇S (M+H+): 1017.44

Found: 1017.4

¹H NMR (400 MHz, CDCl₃)

δ: 8.52 (d, J=2.8 Hz, 1H), 8.12 (s, 1H), 7.72 (d, J=8.3 Hz, 1H), 7.37 (d, J=8.3 Hz, 1H), 7.17 (d, J=2.8 Hz, 1H), 7.09 (s, 1H), 6.73 (d, J=7.2 Hz, 1H), 5.50 (dt, J=7.0, 4.9 Hz, 1H), 4.32 (d, J=12.9 Hz, 1H), 4.03 (dd, J=12.7, 7.8 Hz, 2H), 3.95 (d, J=6.1 Hz, 1H), 3.88 (dd, J=14.6, 7.3 Hz, 1H), 3.73 (d, J=4.8 Hz, 1H), 3.68 (dd, J=10.9, 3.1 Hz, 1H), 3.62 (d, J=11.6 Hz, 1H), 3.43 (d, J=4.8 Hz, 2H), 3.31 (t, J=5.1 Hz, 4H), 2.99 (s, 3H), 2.84 (d, J=14.2 Hz, 1H), 2.61 (t, J=5.0 Hz, 4H), 2.37 (s, 3H), 2.32 (d, J=14.2 Hz, 1H), 2.04-1.95 (m, 1H), 1.85-1.75 (m, 2H), 1.41 (d, J=6.2 Hz, 3H), 1.37 (s, 12H), 1.25 (d, J=7.3 Hz, 2H), 1.21 (d, J=7.1 Hz, 3H), 1.19-1.08 (m, 3H), 1.06 (d, J=6.0 Hz, 3H), 0.90 (s, 3H), 0.85 (s, 3H), 0.61-0.55 (m, 1H).

Part 3—Synthesis of Compound A free base—(1S,2S)—N-[(7S,13S)-21-ethyl-20-{2-[(1S)-1-methoxyethyl]-5-(4-methylpiperazin-1-yl)pyridin-3-yl}-17,17-dimethyl-8,14-dioxo-15-oxa-4-thia-9,21,27,28-tetraazapentacyclo[17.5.2.1\hat{ }{2, 5}.1\hat{ }{9,13}.0\hat{ }{22,26}]octacosa-1(25),2,5(28),19,22(26),23-hexaen-7-yl]-2-methylcyclopropane-1-carboxamide

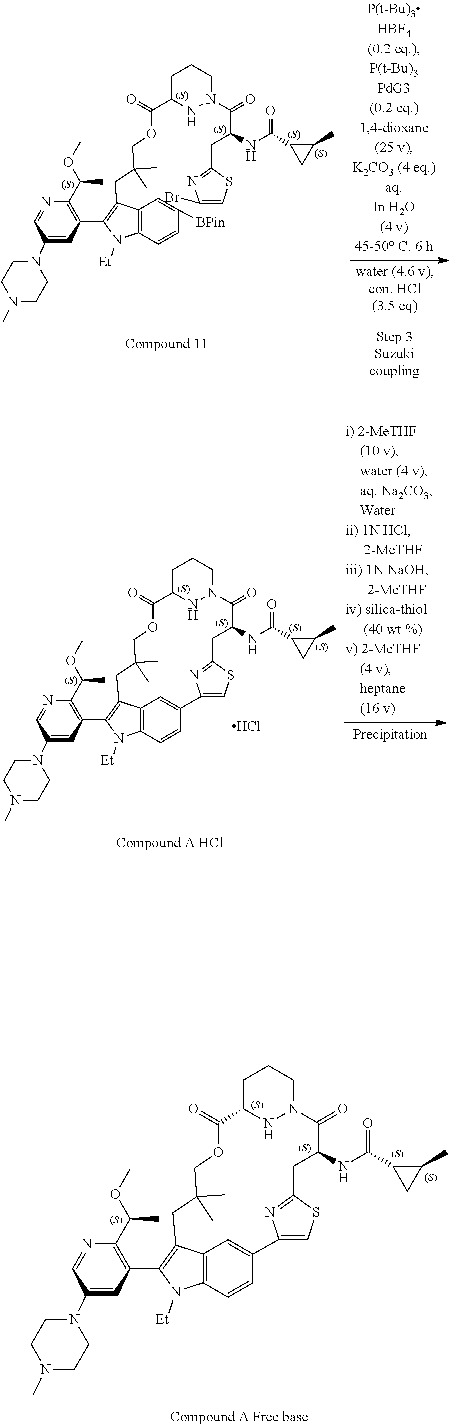

To a reactor was charged 1,4-dioxane (134.65 kg), 2-[(1-ethyl-2-{2-[(1 S)-1-methoxyethyl]-5-(4-methylpiperazin-1-yl)pyridin-3-yl}-5- (tetramethyl-1,3,2-dioxaborolan-2-yl)-1 H-indol-3-yl)methyl]-2-methylpropyl (3S)-1-[(2S)-3-(4-bromo-1,3-thiazol-2-yl)-2-{[(1S,2S)-2-methylcyclopropyl]formamido}propanoyl]-1,2-diazinane-3-carboxylate (Compound 11) (5.50 kg, 81.9% w/w assay, 4.43 mol, 1.0 equiv) and P(t-Bu)₃-HBF₄ (256.5 g, 0.089 mol, 0.2 equiv) at 25° C. Nitrogen was bubbled under surface of mixture for 1 hour at 25° C. To this mixture P(t-Bu)₃ Pd G3 (504.0 g, 0.089 mol, 0.2 equiv) was added. Again, nitrogen was bubbled under surface of mixture for 1 hour at 25° C. Then the resulting reaction mixture was heated to 45° C. A solution of K₂CO₃ (1.22 kg, 8.84 mol, 2.0 equiv) in water (27.00 kg) was added dropwise over a period of an hour. The resulting reaction mixture was stirred at 45˜50° C. for 7 hours. A sample was taken and diluted with MeOH to test the IPC by HPLC analysis (criterion: area % of Compound 11≤3%, actual results: area % of Compound 11=0.1%). Then the reaction mixture was concentrated to 25 L under reduced pressure. The mixture was then diluted with EtOAc (49.0 kg) and water (45.5 kg). The organic phase was separated and the organic phase was washed with water three times (22.8 kg×3). The organic phase was concentrated and co distilled with EtOAc under reduced pressure to remove 1,4-dioxane (GC area % of dioxane: 53%, results: GC area % of dioxane: 1.3%). The resulting product in EtOAc solution was stored to be combined with another two batches for further workup.

Another two batches of Suzuki coupling were carried out following the same procedure shown above. To the combined three batches, water (11.00 kg) and EtOAc (33.50 kg) were added. Then the resulting biphasic solution was cooled to 10° C. To this solution, aqueous HCl was slowly added over a period of 90 minutes at 10° C. (prepared by mixing 13.9 kg of water and 2.4 kg of hydrochloric acid). Then add seeds (136.5 g), the resulting suspension was stirred at 10° C. for another one more hour. The slurry was further cooled down to 0° C. in an hour and held for 1 h. This temperature cycle between 10 to 0° C. was repeated for another 3 times. The suspension was stirred at 0° C. for 10 hours and the resulting slurry was filtered. The filter-cake was washed with water (5.00 kg). The wet cake was suspended in mixed solvents EtOAc (20.0 kg) and water (5.0 kg) at 5° C., stirred for 1 hour. The suspension was filtered and washed with cold-water (5.0 kg, precooled to 5° C.) to give wet Compound A HCl salt as a yellow solid (6.1 kg, 97.6% a/a purity, LOD:29.2%, Pd:595 ppm).

Compound A HCl salt was suspended in mixture of 2-MeTHF (35.70 kg) and water (24.10 kg) at 50 and the aqueous pH was adjusted to 8-9 by adding 10% aq. sodium carbonate solution. Then the organic phase was separated and washed twice with water (2×24 kg). Water (16.1 kg) added to the organic phase and pH was adjusted to 3.7 by 1M. HCl solution. Then the aqueous phase was separated and washed with 2-MeTHF twice (2×13.5 kg). 2-MeTHF (27.7 kg) was added to the aqueous phase and the pH was adjusted to 5.5 by adding 1M aqueous sodium hydroxide solution. The organic phase is separated and washed with water and NaCl solution. SiliaMets Thiol (1.290 kg) was added to the organic phase and the resulting suspension was stirred for 22 hours at 2500. The slurry is filtered and the filter-cake is washed with 2-MeTHF (2×5.5 L). The combined organic phases were concentrated to 12 L under reduced pressure. The resulting MeTHF solution was added to pre-cooled n-heptane (55.80 kg, pre-cooled to −10° C.) at −10° C. over 1 hour. The resulting suspension was stirred for 12 hours at −10° C. The suspension filtered and wet cake was washed with n-heptane twice (2×2.3 kg). The wet cake was dried at 4000 under reduced pressure to afford Compound A free base as an off-white solid (2.46 kg, 99.2% a/a purity, 3.03 mol, 22.8% yield, Table 26).

TABLE 26
HPLC Method for Part 3 of Example 7

| Column: | Eclipse plus C8, 4.6 mm × 150 mm, 3.5 μm | | |
|---|---|---|---|
| Mobile Phase: | A: 0.05% TFA in water | | |
| | B: 0.05% TFA in MeCN | | |
| Gradient: | Time (min) | A % | B % |
| | 0.0 | 65 | 35 |
| | 8.0 | 50 | 50 |
| | 12.0 | 5 | 95 |
| | 16.0 | 5 | 95 |
| | 16.1 | 65 | 35 |

| | |
|---|---|
| Flow Rate: | 0.8 mL/min |
| Post Time | 4.0 min |
| UV Detector Wavelength: | 210 nm |
| Column Temperature: | 40° C. |
| Retention Times: | Compound A: 5.9 min; |
| | Compound 11: 12.5 min |

LCMS (ESI+)

Calculated for C44H58N8O5S (M+H⁺): 811.43

Found: 811.4

¹H NMR (400 MHz, DMSO-d₆)

δ 8.54 (d, J=9.2 Hz, 1H), 8.51 (s, 1H), 8.47 (d, J=2.4 Hz, 1H), 7.78 (s, 1H), 7.73 (dd, J=8.8, 1.2 Hz, 1H), 7.55 (d, J=8.8 Hz, 1H), 7.22 (d, J=2.4 Hz, 1H), 5.60 (t, J=8.8 Hz, 1H), 5.09 (d, J=12.0 Hz, 1H), 4.32-4.15 (m, 5H), 3.60 (br s, 2H), 3.35 (d, J=14.0 Hz, 1H), 3.27 (br s, 4H), 3.22 (s, 3H), 3.19-3.15 (m, 1H), 2.97 (d, J=14.4 Hz, 1H), 2.80-2.74 (m, 1H), 2.49-2.45 (m, 5H), 2.21 (s, 3H), 2.10 (d, J=9.6 Hz, 1H), 1.80 (br s, 2H), 1.56-1.52 (m, 2H), 1.35 (d, J=6.0 Hz, 3H), 1.08 (s, 4H), 0.92-0.89 (m, 7H), 0.56 (d, J=5.2 Hz, 1H), 0.37 (s, 3H).

Part 4—Purification of Compound A—(1S,2S)—N-[(7S,13S)-21-ethyl-20-{2-[(1S)-1-methoxyethyl]-5-(4-methylpiperazin-1-yl)pyridin-3-yl}-17,17-dimethyl-8,14-dioxo-15-oxa-4-thia-9,21,27,28-tetraazapentacyclo[17.5.2.1\hat{ }{2, 5}.1\hat{ }{9,13}.0\hat{ }{22,26}]octacosa-1(25),2,5(28),19,22(26),23-hexaen-7-yl]-2-methylcyclopropane-1-carboxamide

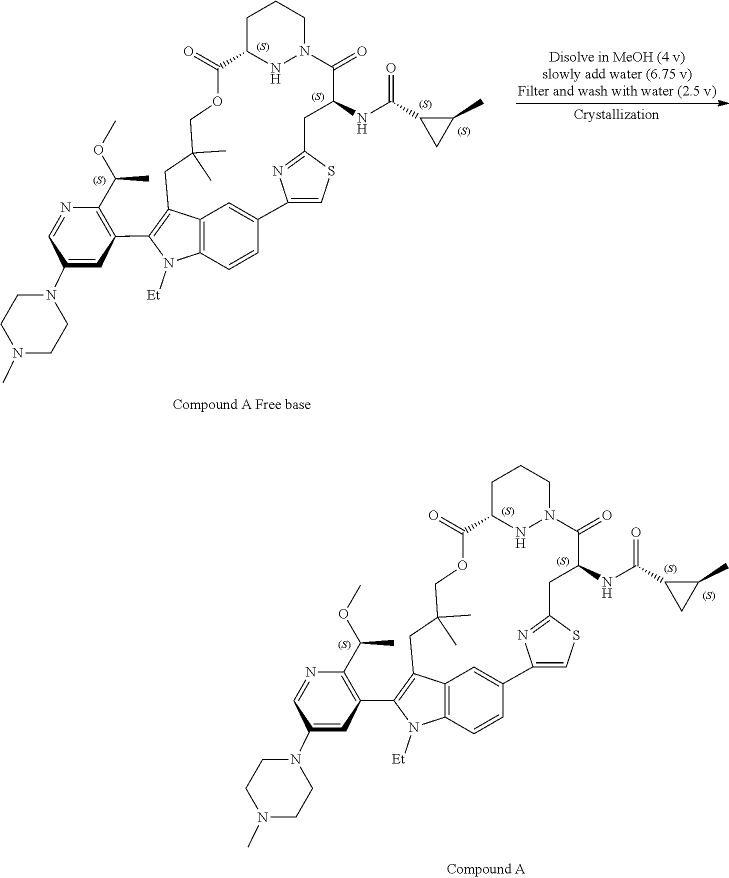

To a reactor was charged MeOH (7.76 kg) and Compound A free base (2.46 kg, 3.03 mol, 1.0 equiv) at 25° C. The resulting suspension was stirred until solids were completely dissolved. The resulting methanol solution was filtered through microporous filter and transferred to another reactor. Then the reactor temperature was maintained at 25° C. and slowly water (2.41 kg, 1.0 V) water was added over a period of 30 minutes. The resulting cloudy solution was stirred for another 30 minutes at 25° C. Then a solution of methanol and water (3.42 kg, 1:2, v/v) slowly over 1 hour. The resulting suspension was stirred for 2 hours at 25° C. Again, to the suspension additional water (2.48 kg) slowly added over 1 hour. The final, suspension was stirred for additional 1 hour. Water (9.29 kg, 3.75 V) was added to the suspension slowly over 2 hours and the mixture was stirred for at least for 16 hours at 25° C. The resulting suspension was filtered and washed with mixed solvent water: MeOH (3:2, v/v) twice (2×2.2 kg), followed by water (4.91 kg) washing. The wet cake was dried under reduced pressure and controlled humidity (temperature: 25±5° C., vacuum≥−0.085 MPa, humidity: 10%-20%) for 37 hours to afford Compound A as a white solid (2.68 kg, 99.4% a/a purity, 93.0% w/w assay, KF: 6.7%, 3.07 mol, 92% yield, Table 27).

TABLE 27
HPLC Method for step 5

| Column: | Poroshell 120 CS-C18, 4.6 mm × 150 mm, 2.7 μm | | |
|---|---|---|---|
| Mobile Phase: | A: 20 mM NH₄HCO₃ in water | | |
| | B: MeCN | | |
| Gradient: | Time (min) | A % | B % |
| | 0.0 | 95 | 10 |
| | 5.0 | 60 | 40 |
| | 45.0 | 60 | 40 |
| | 54.0 | 5 | 95 |
| | 64.0 | 5 | 95 |
| | 65.0 | 90 | 10 |
| | 75.0 | 90 | 10 |

| | |
|---|---|
| Flow Rate: | 0.6 mL/min |
| Post Time | 0.0 min |
| UV Detector | 210 nm |
| Wavelength: | |
| Column | 15° C. |
| Temperature: | |
| Retention Times: | Compound A: 41.1 min |

MS (ESI+)

Calculated for C₄₄H₅₈N₈₀₅S (M+H): 811.43

Found: 811.40

¹H NMR (400 MHz, DMSO-d₆)

δ 8.54 (d, J=9.2 Hz, 1H), 8.51 (s, 1H), 8.47 (d, J=2.4 Hz, 1H), 7.78 (s, 1H), 7.73 (dd, J=8.8, 1.2 Hz, 1H), 7.55 (d, J=8.8 Hz, 1H), 7.22 (d, J=24 Hz, 1H), 5.60 (t, J=8.8 Hz, 1H), 5.09 (d, J=12.0 Hz, 1H), 4.32-4.15 (m, 5H), 3.60 (br s, 2H), 3.35 (d, J=14.0 Hz, 1H), 3.27 (br s, 4H), 3.22 (s, 3H), 3.19-3.15 (m, 1H), 2.97 (d, J=14.4 Hz, 1H), 2.80-2.74 (m, 1H), 2.49-2.45 (m, 5H), 2.21 (s, 3H), 2.10 (d, J=9.6 Hz, 1H), 1.80 (br s, 2H), 1.56-1.52 (m, 2H), 1.35 (d, J=6.0 Hz, 3H), 1.08 (s, 4H), 0.92-0.89 (m, 7H), 0.56 (d, J=5.2 Hz, 1H), 0.37 (s, 3H).

### Example 8. Alternative Synthesis of Compound A

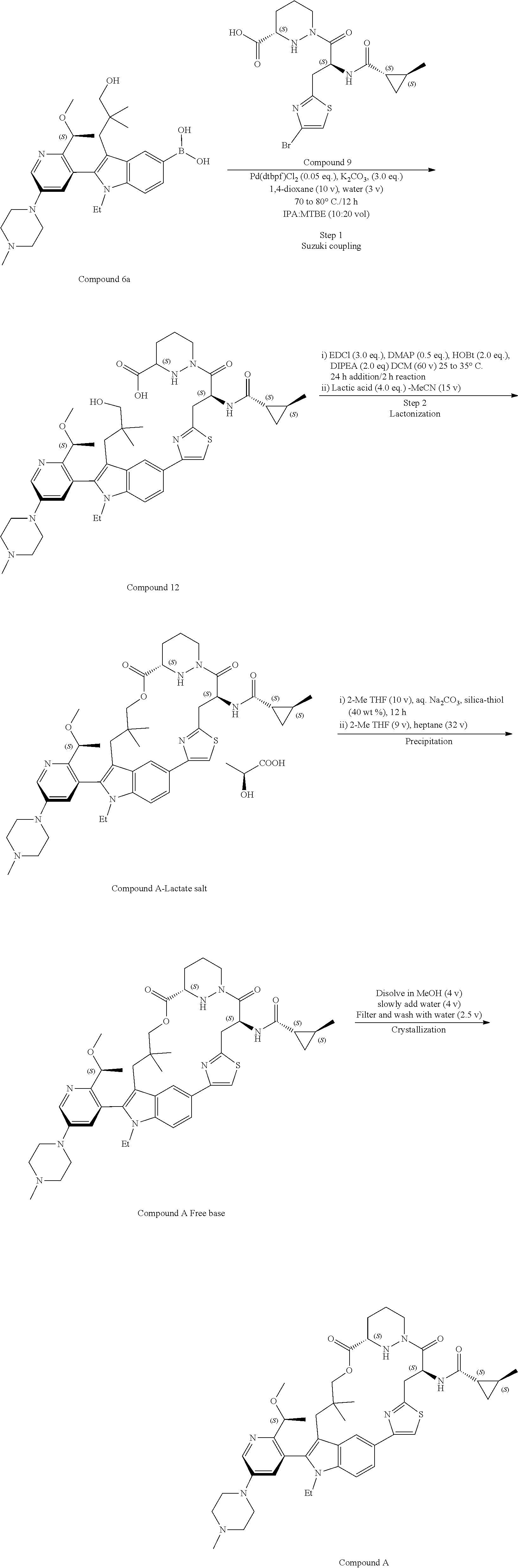

Part 1a—Synthesis of Compound 12—(S)-1-((S)-3-(4-(1-ethyl-3-(3-hydroxy-2,2-dimethylpropyl)-2-(2-((S)-1-methoxyethyl)-5-(4-methylpiperazin-1-yl) pyridin-3-yl)-1H-indol-5-yl)thiazol-2-yl)-2-((1S,2S)-2-methylcyclopropane-1-carboxamido)propanoyl)hexahydropyridazine-3-carboxylic acid

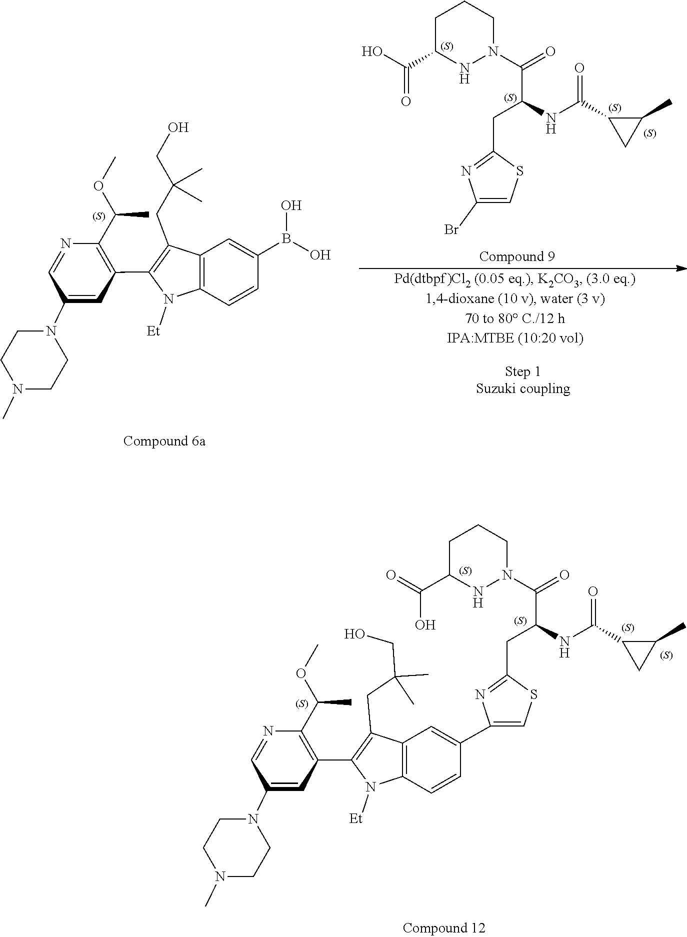

A reactor was charged with (S)-(1-ethyl-3-(3-hydroxy-2,2-dimethylpropyl)-2-(2-(1-methoxyethyl)-5-(4-methylpiperazin-1-yl) pyridin-3-yl)-1H-indol-5-yl)boronic acid (Compound 6a) (54.5 kg 95 wt %, 107.2 mol, 1.0 equiv), (S)-1-((S)-3-(4-bromothiazol-2-yl)-2-((1S,2S)-2-methylcyclopropane-1-carboxamido)propanoyl)hexahydropyridazine-3-carboxylic acid (Compound 9, 49.0 kg, 110.3 mol, 1.03 equiv) and dioxane (514.2 kg). Then anhydrous potassium carbonate (45.4 kg) in purified water (166.5 kg) was added. The mixture was purged with nitrogen for about 1.5 hours. Then [1,1′-bis(di-tert-butylphosphino) ferrocene] dichloropalladium (II) 3.6 kg (0.06-0.07×0.05 eq.) was added into the mixture under nitrogen protection. Spray the reactor with dioxane (70 kg) into the reactor. Again, the reaction mixture was purged with nitrogen for another 1.5 hours. Then the reaction mixture was slowly heated to 75° C. over a period of 4 h. The reaction mixture stirred another 10 h at 75° C. The reaction IPC indicated that the starting material (Compound 6a) was below 1% by HPLC. The reaction mixture was cooled to room temperature and the dark reaction mixture was filtered with 10.2 diatomite to remove insoluble materials. The filtrate cake was washed with 23% aq. NaCl solution (206.9 kg) and dioxane (185 kg). The filtrate was separated, and the organic phase was separated and distilled to 429 L. Water (272 kg) and 2-methyl tetrahydrofuran (242 kg). The basic mixtures were adjusted to pH 9.1 with 7% HCl (25.6 kg), and the aqueous phase was separated. The aqueous phase was acidified using 7% HCl (139 kg) to pH 2 to 3. Then the aqueous phase was washed with MeTHF (277 kg). The aqueous phase was neutralized using 15% aq. sodium carbonate solution (130 kg) to pH 7 to 8. The product was extracted from aqueous phase using DCM-MeOH (2 times 563.4 kg+100 kg). The combined organic phase evaporated and diluted with IPA (275 L) and MeOH (6 kg). MTBE (1274 kg) was added slowly over a period of 5 hours in three portions. During addition product started to crystallize out. The resulting slurry was cooled to 0° C. for 12 h. Then the slurry was filtered, and the wet compound was dried to afford Compound 12 as a gray solid (76.6 kg, 95.9% a/a purity, 87.9 wt %, 76% yield, Table 28).

Part 1b—Alternative synthesis of Compound 12

A reactor was charged with (S)-(1-ethyl-3-(3-hydroxy-2,2-dimethylpropyl)-2-(2-(1-methoxyethyl)-5-(4-methylpiperazin-1-yl) pyridin-3-yl)-1H-indol-5-yl)boronic acid (Compound 6a) (118.4 kg, 233 mol, 1.0 equiv), (S)-1-((S)-3-(4-bromothiazol-2-yl)-2-((1S,2S)-2-methylcyclopropane-1-carboxamido)propanoyl)hexahydropyridazine-3-carboxylic acid (Compound 9, 108.8 kg, 244 mol, 1.04 equiv), and dioxane (1,102 kg). Then anhydrous potassium carbonate (91.7 kg; 5.5 eq.) in purified water (352 kg) was added. The mixture was purged with nitrogen for about 1.5 hours. Then [1,1′-bis(di-tert-butylphosphino) ferrocene] dichloropalladium (II) 7.80 kg (0.065×0.05 eq.) was added into the mixture under nitrogen protection. The reactor was sprayed with dioxane (125 kg). Again, the reaction mixture was purged with nitrogen for another 1.5 hours. Then the reaction mixture was slowly heated to 75° C. over a period of 4 h. The reaction mixture was stirred for another 6 h at 75° C. The reaction IPC indicated that the starting material (Compound 6a) was below 1% by HPLC. The reaction mixture was cooled to room temperature and the dark reaction mixture was filtered with 10.2 diatomite to remove insoluble materials. The filtrate cake was washed with additional dioxane (312 kg). In another reactor, an aqueous sodium chloride solution (103 kg in water (312 kg) and aqueous potassium carbonate solution (80 kg in water (122 kg) were prepared. The filtrate (organic solution) was with a mixture of aq. NaCl and aq. K₂CO₃ solutions. Then, the organic phase separated and concerted to 1188 L (10 V). The resulting solution was co-distilled with IPA (6×1191 L) to reach minimum levels of dioxane and water. The resulting organic phase was adjusted 946 L (8 V). The solution was added to MTBE (3754 L, 31.8 V) slowly over a period of 5 hours. In addition, the product started to crystallize/precipitate as a slurry. The resulting slurry was cooled to 0° C. for 12 h. Then, the slurry was filtered, and the wet compound was dried to afford the sodium salt of Compound 12 as a gray solid (184.6 kg, 96.1% a/a purity, 87.7 wt %, 84% yield).

TABLE 28
HPLC Method for Compound 12

| Column: | Waters xbridge C18, 4.6 mm × 150 mm, 3.5 μm | | |
|---|---|---|---|
| Mobile Phase: | A: 10 mM NH₄OAc in water | | |
| | B: ACN | | |
| Gradient: | Time (min) | A % | B % |
| | 0.0 | 85 | 15 |
| | 6.0 | 60 | 40 |
| | 9.0 | 60 | 40 |
| | 12.0 | 35 | 65 |
| | 15.0 | 20 | 80 |
| | 18.0 | 20 | 80 |
| | 18.1 | 85 | 15 |
| | 23.0 | 85 | 15 |

| | |
|---|---|
| Flow Rate: | 1 mL/min |
| UV Detector | 210 nm |
| Wavelength: | |
| Column Temperature: | 40° C. |
| Retention Times: | Compound 12: 7.44 min |

LCMS (ESI+)

Calculated for C₄₄H₆₀N₈O₆S (M+H⁺): 829.44

Found: 829.90

¹H NMR (400 MHz, DMSO-d₆)

δ ppm 8.44 (d, J=2.81 Hz, 1H), 8.32 (s, 1H), 8.05 (d, J=8.19 Hz, 1H), 7.74-7.71 (m, 2H), 7.51-7.49 (m, 1H), 7.30-7.27 (m, 1H), 5.53-5.46 (m, 1H), 4.53-4.50 (m, 1H), 4.20 (br d, J=12.72 Hz, 1H), 4.10-4.01 (m, 2H), 3.92-3.84 (m, 1H), 3.28-3.20 (m, 7H), 3.12 (br d, J=10.72 Hz, 2H), 3.08 (s, 3 H), 3.04-3.01 (m, 1H), 2.88 (s, 3H), 2.84 (m, 1H), 2.72 (s, 1H), 2.69-2.62 (m, 2H), 2.53-2.52 (m, 1H), 2.46 (br t, J=4.77*(2) Hz, 5H), 2.26-2.24 (m, 1H), 2.21-2.16 (m, 4H), 1.93-1.87 (m, 1H), 1.68 (br dd, J=9.11, 3.00 Hz, 1H), 1.58 (d, J=4.03 Hz, 1H), 144 (d, J=6.68 Hz, 1H), 1.36-1.34 (m, 2H), 1.15 (t, J=7.15 Hz, 2H), 0.93-0.85 (m, 1H), 0.69-0.63 (m, 3H), 0.63-0.58 (m, 3H), 0.53-0.45 (m, 1H)

Part 2a—Synthesis of Compound A Lactate Salt—(1S,2S)—N-((63S,4S,Z)-11-ethyl-12-(2-((S)-1-methoxyethyl)-5-(4-methylpiperazin-1-yl)pyridin-3-yl)-10,10-dimethyl-5,7-dioxo-61,62,63,64,65,66-hexahydro-11H-8-oxa-2(4,2)-thiazola-1(5,3)-indola-6(1,3)-pyridazinacycloundecaphane-4-yl)-2-methylcyclopropane-1-carboxamide lactate salt

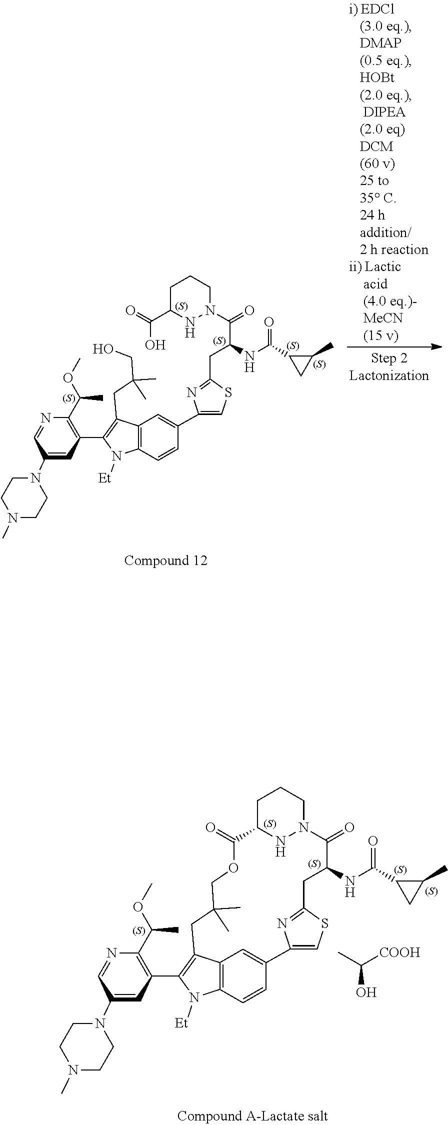

To reactor-1 was charged 1-Hydroxy-1H-benzotriazole (21.1 kg, 156 mol, 2.0 eq) and 4-dimethylaminopyidine (4.9 kg, 0.5 eq) and N,N-diisopropylethylamine (21 kg, 2.0 eq) and 1-Ethyl-3-(3-dimethyllaminopropyl)carbodiimide-HCl (45.5 kg, 237 mol, 3.0 eq) and dissolved in DCM (3601.3 kg). After (S)-1-((S)-3-(4-(1-ethyl-3-(3-hydroxy-2,2-dimethylpropyl)-2-(2-((S)-1-methoxyethyl)-5-(4-methylpiperazin-1-yl) pyridin-3-yl)-1 H-indol-5-yl) thiazol-2-yl)-2-((1S,2S)-2-methylcyclopropane-1-carboxamido)propanoyl)hexahydropyridazine-3-carboxylic acid (Compound 12, 65.7 kg, 87.9 wt %, 79 mol, 1.0 eq) dissolved in DCM (920 kg) in reactor 2. Transfer the solution from reactor-2 into reactor-1 at 25-35° C. over 25 h. The solution was stirred at 25-35° C. for 2.0 hr. After reaction completing, quench the mixture with water (330 kg) and concentrate the solution to 2000 kg. Charge water (330 kg) into the mixture and separate the organic phase. Washing the organic phase with water (720 kg) twice, 1708.8 kg organic phase was concentrated to 175 kg and co-evaporated with acetonitrile (460 kg) 2 times. The concentration in acetonitrile was adjusted to 300 kg. Then 98% lactic acid (26.2 kg, 4.0 equiv) was added slowly over 2 hours. Water (3.8 kg) was added to the solution. Then seeds (0.54 kg) were added and the resulting slurry was stirred at 25° C. for 12 hours, then cooled to 0° C. for 5 hours and stirred at 0° C. for 20 hours. The compound was isolated after filtration and drying. The crude wet-cake was slurried in MeCN (340 kg) at 0° C. for 18 hours. Then the slurry was filtered and dried to afford Compound A lactate salt as a gray solid (35.6 kg, 99.0% a/a purity, 82.2% wt %, 48% yield, Table 29).

Part 2b—Synthesis of Compound A Lactate Salt—(1S,2S)—N-((63S,4S,Z)-11-ethyl-12-(2-((S)-1-methoxyethyl)-5-(4-methylpiperazin-1-yl)pyridin-3-yl)-10,10-dimethyl-5,7-dioxo-61,62,63,64,65,66-hexahydro-11H-8-oxa-2(4,2)-thiazola-1(5,3)-indola-6(1,3)-pyridazinacycloundecaphane-4-yl)-2-methylcyclopropane-1-carboxamide lactate salt

To reactor-1 was charged 1-Hydroxy-1H-benzotriazole (24 kg, 177 mol, 2.0 eq), 4-dimethylaminopyidine (5.6 kg, 45.9 mol, 0.5 eq), N,N-diisopropylethylamine (24 kg, 186 mol, 2.0 eq), and 1-Ethyl-3-(3-dimethyllaminopropyl)carbodiimide-HCl (52 kg, 272 mol, 3.0 eq). The reagents were subsequently dissolved in DCM (4190 kg). In another reactor (reactor 2), (S)-1-((S)-3-(4-(1-ethyl-3-(3-hydroxy-2,2-dimethylpropyl)-2-(2-((S)-1-methoxyethyl)-5-(4-methylpiperazin-1-yl) pyridin-3-yl)-1 H-indol-5-yl) thiazol-2-yl)-2-((1 S,2S)-2-methylcyclopropane-1-carboxamido)propanoyl)hexahydropyridazine-3-carboxylic acid (Compound 12, 75.3 kg, 90.8 mol, 1.0 eq) was dissolved in DCM (1000 kg). Then, the solution from reactor-2 was transferred into reactor-1 at 25-35° C. over 25 h. The solution was stirred at 25-35° C. for 2 h. Upon reaction completion, the mixture was quenched with water (350 kg) and was subsequently concentrated to about 2000 kg. Water (350 kg) was added to the mixture and the organic phase was separated. Water (˜750 kg) was added to the organic phase, and the aqueous phase pH was adjusted to 4 to 5 using 85% lactic acid. The organic phase was separated and concentrated to 232 L and co-evaporated with acetonitrile (740 L) three times. The resulting organic phase volume was adjusted to about 300 kg. Then 85% lactic acid (16.2 kg, 2.0 equiv) was added slowly over 2 hours, and water (2.4 kg) was added to the solution. Then seeds of Compound 12 (0.23 kg) were added, and the resulting slurry was stirred at 25° C. for 12 hours, then cooled to 0° C. for 5 hours and stirred at 0° C. for 20 hours. Compound 12 was isolated after filtration. The crude wet cake was recrystallized again in MeCN (˜340 kg), heated to 60° C., and cooled to 0° C. for 20 hours. Then, the slurry was filtered and dried to afford Compound 12 L-lactate salt as a gray solid (57.07 kg, 99.0% a/a purity, 81.1% wt %, 63% yield).

TABLE 29
HPLC Method for Compound A lactic salt

| Column: | Agilent poroshell 120 CS-C18 C18, | | |
|---|---|---|---|
| | 4.6 × 150 mm, 2.7 μm | | |
| Mobile Phase: | A: 20 mM NH₄HCO₃ in water | | |
| | B: ACN | | |
| Gradient: | Time (min) | A % | B % |
| | Initial | 95 | 10 |
| | 5.00 | 60 | 40 |
| | 45.00 | 60 | 40 |
| | 54.00 | 5 | 95 |
| | 64.00 | 5 | 95 |
| | 65.00 | 90 | 10 |
| | 75.00 | 90 | 10 |

| | |
|---|---|
| Flow Rate: | 0.6 mL/min |
| UV Detector | 210 nm |
| Wavelength: | |
| Column Temperature: | 15° C. |
| Retention Times: | Compound A: 46.4 min |

LCMS (ESI+).

Calculated for C₄₄H₅₈N₈O₅S+C₃H₆O₃ (M+H⁺): 901.14 (as lactate salt)

Found: 811.3

¹H NMR (400 MHz, CDCl₃):

δ ppm: 8.48-8.52 (m, 1H), 8.39-8.45 (m, 1H), 7.49-7.55 (m, 1H), 7.24-7.29 (m, 1H), 7.03 (d, J=2.50 Hz, 1H), 6.40-6.55 (m, 1H), 5.80-5.91 (m, 1H), 5.15-5.22 (m, 1H), 5.12-5.27 (m, 1H), 4.47-4.56 (m, 1H), 4.14-4.29 (m, 4H) 4.01-4.12 (m, 4H), 3.90-3.97 (m, 2H), 3.71-3.77 (m, 1 H), 3.60-3.68 (m, 1H), 3.35-3.40 (m, 4H), 3.27-3.33 (m, 3H), 3.00-3.12 (m, 2H), 2.91-2.96 (m, 3H), 2.57-2.66 (m, 1H), 2.47-2.55 (m, 3H), 2.32-2.41 (m, 1H), 2.07-2.18 (m, 1H), 1.85-1.91 (m, 1H), 1.68-1.79 (m, 1H), 1.48-1.58 (m, 1H), 1.31-1.39 (m, 6H), 1.09-1.22 (m, 3H), 0.87-0.92 (m, 3H), 0.78-0.86 (m, 3H), 0.52-0.60 (m, 1H), 0.48-0.51 (m, 1H), 0.47-0.51 (m, 1H), 0.31-0.40 (m, 3H).

Part 3a—Synthesis of Compound A Free Base—(1S,2S)—N-((63S,4S,Z)-11-ethyl-12-(2-((S)-1-methoxyethyl)-5-(4-methylpiperazin-1-yl)pyridin-3-yl)-10,10-dimethyl-5,7-dioxo-61,62,63,64,65,66-hexahydro-11H-8-oxa-2(4,2)-thiazola-1(5,3)-indola-6(1,3)-pyridazinacycloundecaphane-4-yl)-2-methylcyclopropane-1-carboxamide

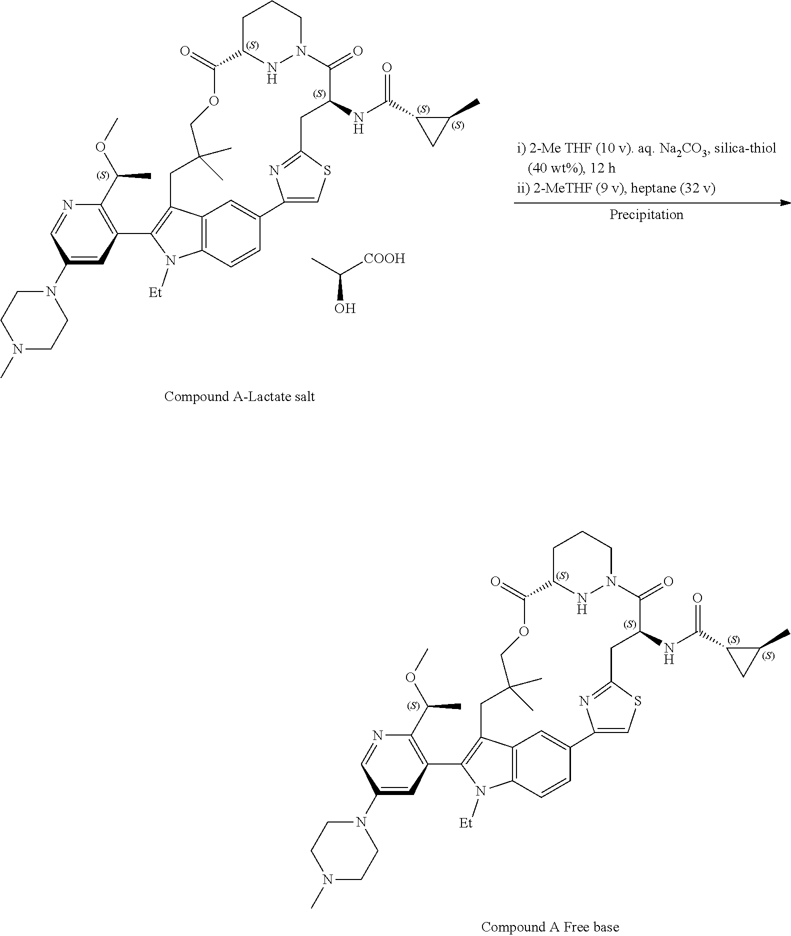

To a reactor was charged (1S,2S)—N-((63S,4S,Z)-11-ethyl-12-(2-((S)-1-methoxyethyl)-5-(4-methylpiperazin-1-yl)pyridin-3-yl)-10,10-dimethyl-5,7-dioxo-61,62,63,64,65,66-hexahydro-11 H-8-oxa-2(4,2)-thiazola-1(5,3)-indola-6(1,3)-pyridazinacycloundecaphane-4-yl)-2-methylcyclopropane-1-carboxamide lactate salt (28.0 kg, 34.5 mol, 1.0 eq) in 2-MeTHF (292 kg) and water (113 kg). The mixture was cooled to 0-10° C. Then aq 15% sodium carbonate solution (15 kg) was slowly added to reactor to neutralize the pH to 8˜9 at 0-10° C. The mixture was stirred for 30 minutes, and the organic phase was separated. The organic phase was washed with water (225 kg). Then it was washed with 25% NaCl (120 kg). To the organic phase, silica thiol 11.2 kg (0.38-0.42 X) was added and the slurry, which was stirred for 12 hours at room temperature to remove residual palladium. Then, the silica-thiol was removed by filtration. The filtrate was concentrated to 112 L. This solution was added to heptane (1200 kg) over a period of 4 hours. The resulting slurry was filtered and the filter-cake dried to afford crude Compound A free base as a white solid (24.9 kg, 98.9% a/a purity, 96.4% w/w 85.6% yield, Table 30).

Part 3b—Synthesis of Compound A Free Base—(1S,2S)—N-((63S,4S,Z)-11-ethyl-12-(2-((S)-1-methoxyethyl)-5-(4-methylpiperazin-1-yl)pyridin-3-yl)-10,10-dimethyl-5,7-dioxo-61,62,63,64,65,66-hexahydro-11H-8-oxa-2(4,2)-thiazola-1(5,3)-indola-6(1,3)-pyridazinacycloundecaphane-4-yl)-2-methylcyclopropane-1-carboxamide

To a reactor was charged (1S,2S)—N-((63S,4S,Z)-11-ethyl-12-(2-((S)-1-methoxyethyl)-5-(4-methylpiperazin-1-yl)pyridin-3-yl)-10,10-dimethyl-5,7-dioxo-61,62,63,64,65,66-hexahydro-11 H-8-oxa-2(4,2)-thiazola-1(5,3)-indola-6(1,3)-pyridazinacycloundecaphane-4-yl)-2-methylcyclopropane-1-carboxamide lactate salt (Compound A lactate salt, 49.6 kg, 55 mol, 1.0 eq) in 2-MeTHF (440 kg, 8.9 V) and water (198 kg). The mixture was cooled to 0-10° C. Then aq. 15% sodium carbonate solution (27 kg) was slowly added to the reactor to neutralize the pH to 8-9 at 0-10° C. The mixture was stirred for 30 minutes, and the organic phase was separated. The organic phase was washed with water (398 kg), followed by 25% NaCl (198 kg). To the organic phase, silica thiol 11.2 kg (0.38-0.42 X) or 3-Mercaptopropyl ethyl sulphide silica (SPM32) was added and the slurry was stirred for 12 hours at room temperature to remove residual palladium. Then, the silica-thiol was removed by filtration. The filtrate was concentrated to 349 L (7 V). This solution was added to heptane (1857 L, 37.4 V) over a period of 4 hours. The resulting slurry was filtered, and the filter cake was dried to afford crude Compound A free base as a white solid (44.2 kg, 99.6% a/a purity, 94.9% w/w 83% yield).

TABLE 30
HPLC Method for Compound A (crude)

| Column: | Agilent poroshell 120 CS-C18 C18, | | |
|---|---|---|---|
| | 4.6 × 150 mm, 2.7 μm | | |
| Mobile Phase: | A: 20 mM NH₄HCO₃ in water | | |
| | B: ACN | | |
| Gradient: | Time (min) | A % | B % |
| | Initial | 95 | 10 |
| | 5.00 | 60 | 40 |
| | 45.00 | 60 | 40 |
| | 54.00 | 5 | 95 |
| | 64.00 | 5 | 95 |
| | 65.00 | 90 | 10 |
| | 75.00 | 90 | 10 |

| | |
|---|---|
| Flow Rate: | 0.6 mL/min |
| UV Detector Wavelength: | 210 nm |
| Column Temperature: | 15° C. |
| Retention Times: | Compound A: 46.4 min |

LCMS (ESI+)

Calculated for C₄₄H₅₈N₈O₅S (M+H⁺): 811.43

Found: 811.3

¹H NMR (400 MHz, DMSO-d₆)

δ 8.54 (d, J=9.2 Hz, 1H), 8.51 (s, 1H), 8.47 (d, J=2.4 Hz, 1H), 7.78 (s, 1H), 7.73 (dd, J=8.8, 1.2 Hz, 1H), 7.55 (d, J=8.8 Hz, 1H), 7.22 (d, J=2.4 Hz, 1H), 5.60 (t, J=8.8 Hz, 1H), 5.09 (d, J=12.0 Hz, 1H), 4.32-4.15 (m, 5H), 3.60 (br s, 2H), 3.35 (d, J=14.0 Hz, 1H), 3.27 (br s, 4H), 3.22 (s, 3H), 3.19-3.15 (m, 1H), 2.97 (d, J=14.4 Hz, 1H), 2.80-2.74 (m, 1H), 2.49-2.45 (m, 5H), 2.21 (s, 3H), 2.10 (d, J=9.6 Hz, 1H), 1.80 (br s, 2H), 1.56-1.52 (m, 2H), 1.35 (d, J=6.0 Hz, 3H), 1.08 (s, 4H), 0.92-0.89 (m, 7H), 0.56 (d, J=5.2 Hz, 1H), 0.37 (s, 3H).

Part 4a—Compound A—(1S,2S)—N-((63S,4S,Z)-11-ethyl-12-(2-((S)-1-methoxyethyl)-5-(4-methylpiperazin-1-yl)pyridin-3-yl)-10,10-dimethyl-5,7-dioxo-61,62,63,64,65,66-hexahydro-11H-8-oxa-2(4,2)-thiazola-1(5,3)-indola-6(1,3)-pyridazinacycloundecaphane-4-yl)-2-methylcyclopropane-1-carboxamide

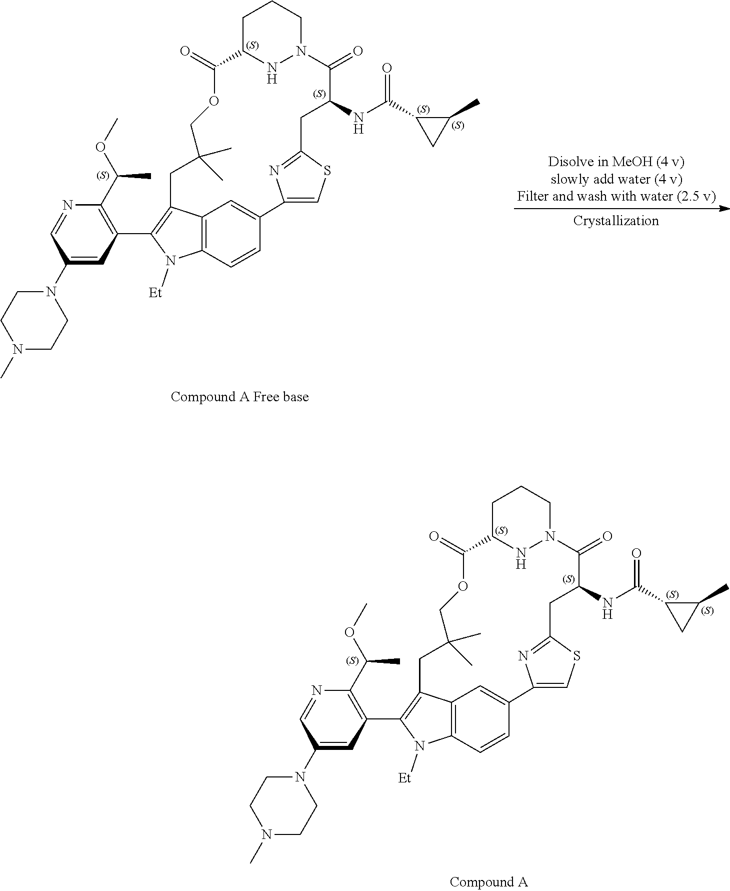

To a reactor 1, (1S,2S)—N-((63S,4S,Z)-11-ethyl-12-(2-((S)-1-methoxyethyl)-5-(4-methylpiperazin-1-yl)pyridin-3-yl)-10,10-dimethyl-5,7-dioxo-61,62,63,64,65,66-hexahydro-11 H-8-oxa-2(4,2)-thiazola-1(5,3)-indola-6(1,3)-pyridazinacycloundecaphane-4-yl)-2-methylcyclopropane-1-carboxamide (Compound A free base, 23.9 kg, 29.47 mol, 1.0 eq) and MeOH (76 kg) were charged. To the resulting solution, purified water (29 kg) was added dropwise into Reactor 2 for 3 h at 20-30° C. Then, add 0.29 kg seed and stir at 20-30° C. for 2-4 h. To the cloudy solution add additional purified water dropwise (67 kg) into Reactor 2 for 4-6 h at 20-30° C. Then the resulting slurry was stirred at 20-30° C. for 8-12 hours. The slurry was filtered and the wet-cake was dried to afford crude Compound A as a white solid (23.9 kg, 99.4% a/a purity, 96% w/w assay, 96% yield, Table 31).

Part 4b—Compound A—(1S,2S)—N-((63S,4S,Z)-11-ethyl-12-(2-((S)-1-methoxyethyl)-5-(4-methylpiperazin-1-yl)pyridin-3-yl)-10,10-dimethyl-5,7-dioxo-61,62,63,64,65,66-hexahydro-11H-8-oxa-2(4,2)-thiazola-1(5,3)-indola-6(1,3)-pyridazinacycloundecaphane-4-yl)-2-methylcyclopropane-1-carboxamide

(1S,2S)—N-((63S,4S,Z)-11-ethyl-12-(2-((S)-1-methoxyethyl)-5-(4-methylpiperazin-1-yl)pyridin-3-yl)-10,10-dimethyl-5,7-dioxo-61,62,63,64,65,66-hexahydro-11 H-8-oxa-2(4,2)-thiazola-1(5,3)-indola-6(1,3)-pyridazinacycloundecaphane-4-yl)-2-methylcyclopropane-1-carboxamide (Compound A free base, 41.9 kg, 51.7 mol, 1.0 eq) and MeOH (166 kg) were charged to reactor 1. Purified water (50 kg) was added dropwise into the resulting solution for 3 h at 20-30° C. Then, 0.42 kg seed was added to the mixture and the resulting slurry was stirred at 20-30° C. for 5 h. Purified water (116 kg) was added dropwise for 4-6 h at 20-30° C. Then, the resulting slurry was stirred at 20-30° C. for 16-24 hours. The slurry was filtered, and the wet cake was washed with MeOH and water (30 kg; 34 kg). The wet cake was dried under nitrogen flow with 35-55% relative humidity to afford Compound A as a white solid (44.12 kg, 99.7% a/a purity, 92.5% w/w assay, 97% yield).

TABLE 31
HPLC Method for Compound A

| Column: | Agilent poroshell 120 CS-C18 C18, | | |
|---|---|---|---|
| | 4.6 × 150 mm, 2.7 μm | | |
| Mobile Phase: | A: 20 mM NH₄HCO₃ in water | | |
| | B: ACN | | |
| Gradient: | Time (min) | A % | B % |
| | Initial | 95 | 10 |
| | 5.00 | 60 | 40 |
| | 45.00 | 60 | 40 |
| | 54.00 | 5 | 95 |
| | 64.00 | 5 | 95 |
| | 65.00 | 90 | 10 |
| | 75.00 | 90 | 10 |

| | |
|---|---|
| Flow Rate: | 0.6 mL/min |
| UV Detector Wavelength: | 210 nm |
| Column Temperature: | 15° C. |
| Retention Times: | Compound A: 46.4 min |

LCMS (ESI+)

Calculated for C₄₄H₅₈N₈O₅S (M+H⁺): 811.43

Found: 811.3

¹H NMR (400 MHz, DMSO-d₆)

δ 8.54 (d, J=9.2 Hz, 1H), 8.51 (s, 1H), 8.47 (d, J=2.4 Hz, 1H), 7.78 (s, 1H), 7.73 (dd, J=8.8, 1.2 Hz, 1H), 7.55 (d, J=8.8 Hz, 1H), 7.22 (d, J=2.4 Hz, 1H), 5.60 (t, J=8.8 Hz, 1H), 5.09 (d, J=12.0 Hz, 1H), 4.32-4.15 (m, 5H), 3.60 (br s, 2H), 3.35 (d, J=14.0 Hz, 1H), 3.27 (br s, 4H), 3.22 (s, 3H), 3.19-3.15 (m, 1H), 2.97 (d, J=14.4 Hz, 1H), 2.80-2.74 (m, 1H), 2.49-2.45 (m, 5H), 2.21 (s, 3H), 2.10 (d, J=9.6 Hz, 1H), 1.80 (br s, 2H), 1.56-1.52 (m, 2H), 1.35 (d, J=6.0 Hz, 3H), 1.08 (s, 4H), 0.92-0.89 (m, 7H), 0.56 (d, J=5.2 Hz, 1H), 0.37 (s, 3H).

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features set forth herein.

All publications, patents and patent applications are herein incorporated by reference in their entirety to the same extent as if each individual publication, patent, or patent application was specifically and individually indicated to be incorporated by reference in its entirety.

1. A method of preparing Compound 1: 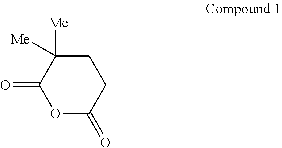
the method comprising:
a) reacting Compound 1a and Compound 1b to form Compound 1c: 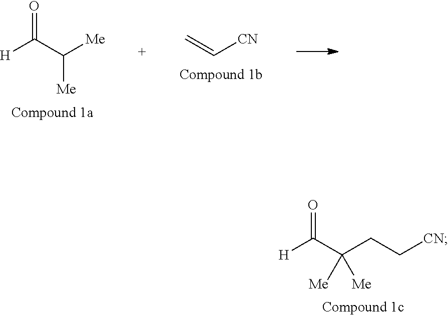
b) oxidizing and hydrolyzing Compound 1c to form Compound 1d: 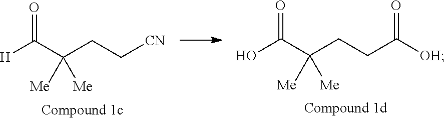 [SMILES: CC(C)(C=O)CCC#N ; CC(C)(CCC(=O)O)C(=O)O]
and
c) cyclizing Compound 1d to form Compound 1:

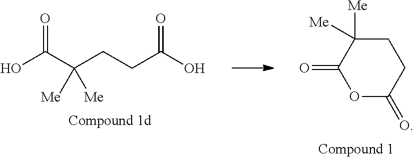 [SMILES: CC(C)(CCC(=O)O)C(=O)O ; CC1(C)CCC(=O)OC1=O]

2. The method of claim 1, wherein the oxidizing and hydrolyzing step (b) comprises a first step of oxidizing Compound 1c to Compound 1e and a second step of hydrolyzing Compound 1e to Compound 1d:

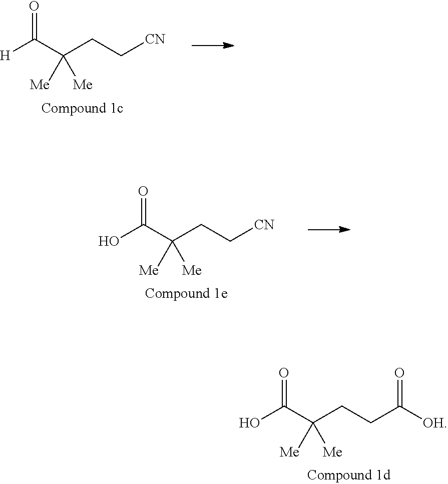 [SMILES: CC(C)(C=O)CCC#N ; CC(C)(CCC#N)C(=O)O ; CC(C)(CCC(=O)O)C(=O)O]

3. A compound having the structure of Formula II: 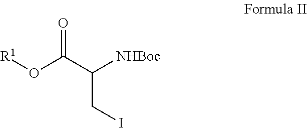
or a salt thereof, wherein R¹ is optionally substituted C₁-C₆ alkyl, optionally substituted 3- to 10-membered cycloalkyl, or optionally substituted C₆-C₁₀ aryl.

4. The compound of claim 3, wherein the compound has the structure of Formula IIa: 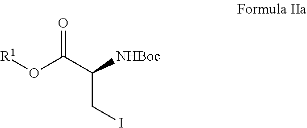
or a salt thereof.

5. A method of preparing Compound 2a, the method comprising:
a) esterifying Compound 2b to form Compound 2c: 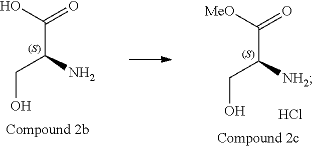
b) protecting and tosylating Compound 2c to form Compound 2d: 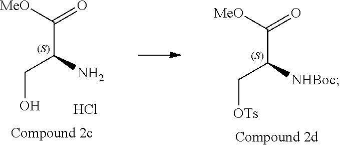
and
c) iodinating Compound 2d to form Compound 2a:

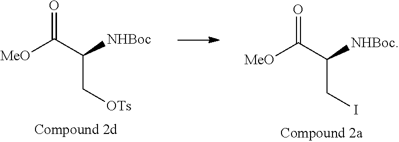 [SMILES: COC(=O)[C@H](COS(=O)(=O)c1ccc(C)cc1)NC(=O)OC(C)(C)C ; COC(=O)[C@H](CI)NC(=O)OC(C)(C)C]

6. The method of claim 5, wherein the protecting and tosylating step (b) comprises a first step of protecting Compound 2c to form Compound 2e and a second step of tosylating Compound 2e to form Compound 2d:

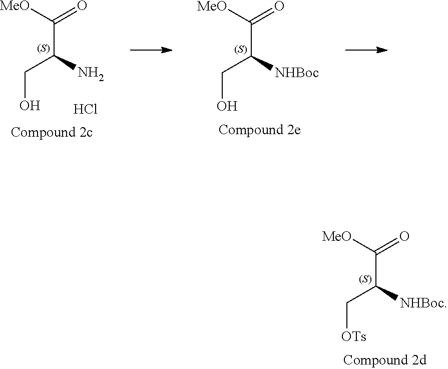

7. A method of preparing Compound 3: 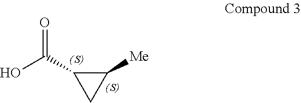
the method comprising:
a) contacting Compound 3a and Compound 3b in the presence of a base to form Compound 3c: 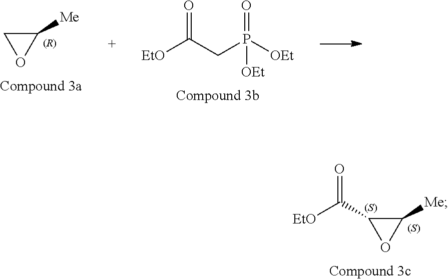
and
b) hydrolyzing Compound 3c to form Compound 3:

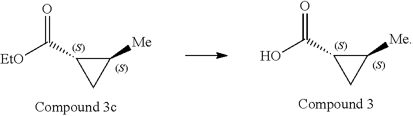

8. A compound having the structure of Formula III, Compound 5, or Compound 6: 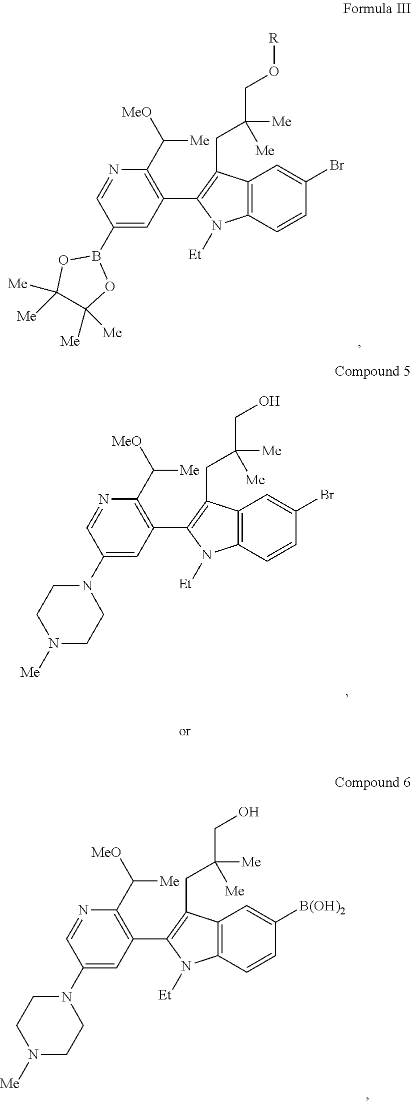
or a salt thereof, wherein R is H or

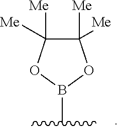 [SMILES: *B1OC(C)(C)C(C)(C)O1]

9. A method of preparing Compound 6a, the method comprising:
a) borylating Compound 7 to form Compound 4a: 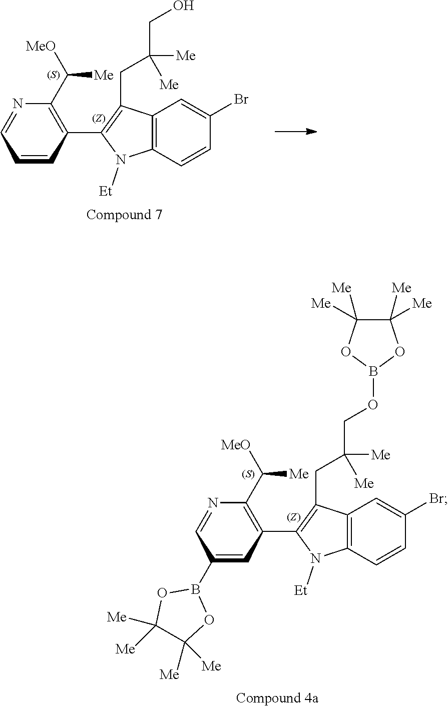
b) coupling Compound 4a and Compound 6b to form Compound 5a: 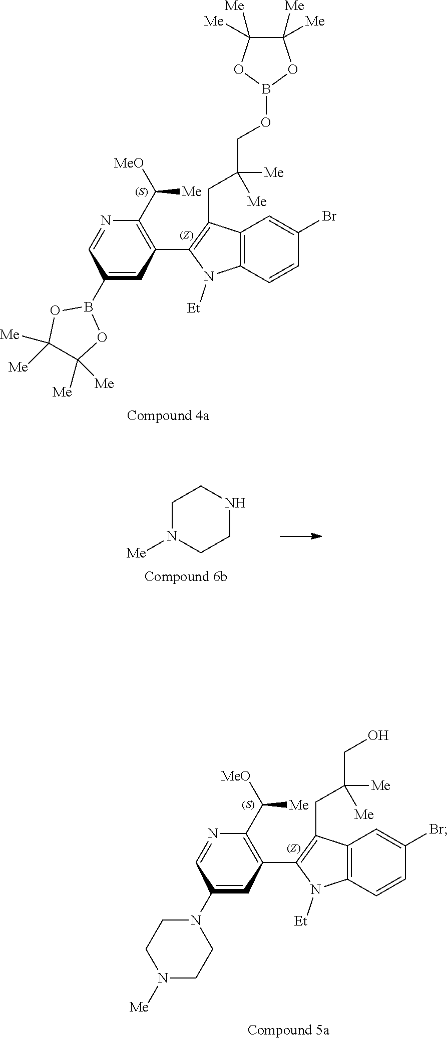
and
c) borylating Compound 5a to form Compound 6a:

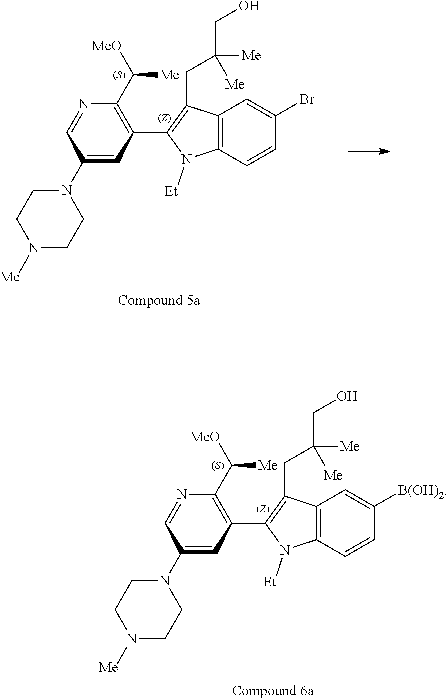

10. A compound having the structure of Formula I: 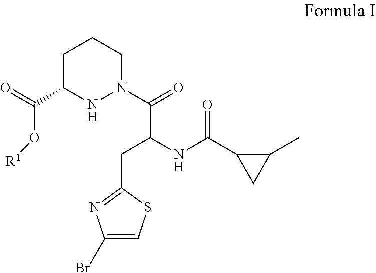
or a salt thereof, wherein R¹ is H or C₁-C₆ alkyl.

11. A method of preparing Compound 9c: 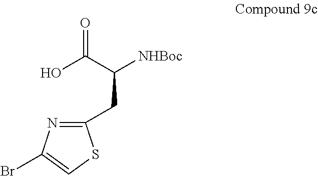 [SMILES: CC(C)(C)OC(=O)N[C@@H](Cc1nc(Br)cs1)C(=O)O]
or a salt thereof, the method comprising:
a) formylating Compound 9c-1 to form Compound 9c-2: 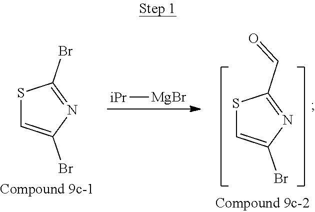
b) condensing Compound 9c with malonic acid to form Compound 9c-3: 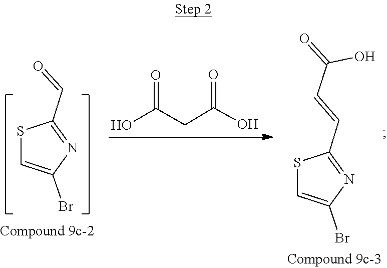
c) aminating Compound 9c-3 to form Compound 9c-4 H₂O: 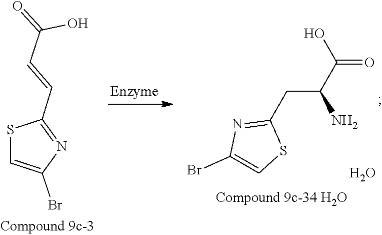 [SMILES: O=C(O)/C=C/c1nc(Br)cs1 ; N[C@@H](Cc1nc(Br)cs1)C(=O)O ; O]
and
d) protecting Compound 9c-4 to form Compound 9c:

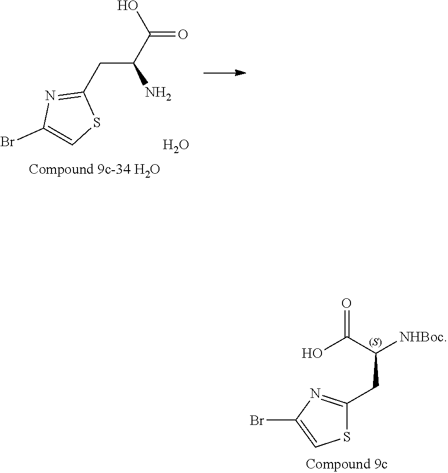

12. A method of preparing Compound 9: 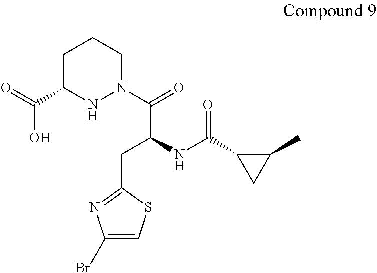 [SMILES: C[C@H]1C[C@@H]1C(=O)N[C@@H](Cc1nc(Br)cs1)C(=O)N1CCC[C@@H](C(=O)O)N1]
the method comprises:
a) coupling Compound 2a with Compound 9a to form Compound 9b: 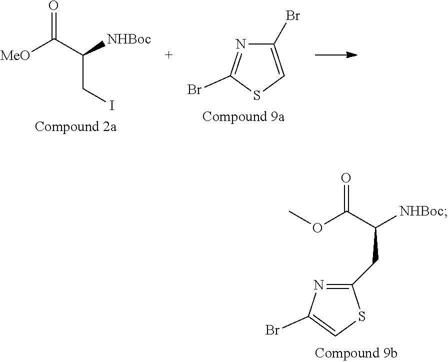 [SMILES: COC(=O)[C@H](CI)NC(=O)OC(C)(C)C ; Brc1csc(Br)n1 ; COC(=O)[C@H](Cc1nc(Br)cs1)NC(=O)OC(C)(C)C]
b) hydrolyzing Compound 9b to form Compound 9c: 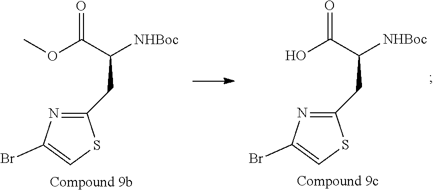 [SMILES: COC(=O)[C@H](Cc1nc(Br)cs1)NC(=O)OC(C)(C)C ; CC(C)(C)OC(=O)N[C@@H](Cc1nc(Br)cs1)C(=O)O]
c) coupling Compound 9c and Compound 9d to form Compound 9e: 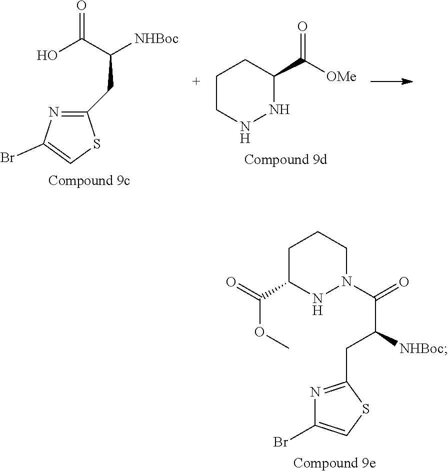 [SMILES: CC(C)(C)OC(=O)N[C@@H](Cc1nc(Br)cs1)C(=O)O ; COC(=O)[C@@H]1CCCNN1 ; COC(=O)[C@@H]1CCCN(C(=O)[C@H](Cc2nc(Br)cs2)NC(=O)OC(C)(C)C)N1]
d) deprotecting Compound 9e to form Compound 9f: 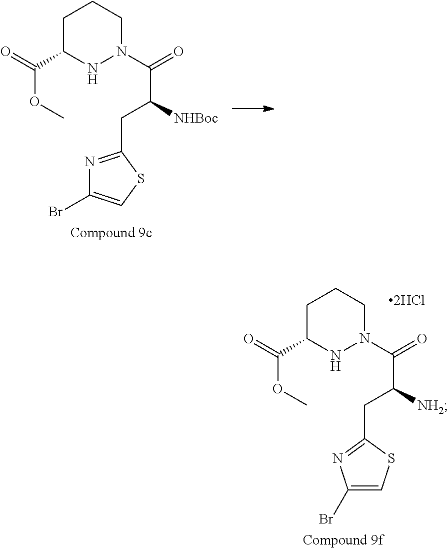 [SMILES: COC(=O)[C@@H]1CCCN(C(=O)[C@H](Cc2nc(Br)cs2)NC(=O)OC(C)(C)C)N1 ; COC(=O)[C@@H]1CCCN(C(=O)[C@@H](N)Cc2nc(Br)cs2)N1]
e) coupling Compound 9f with Compound 3 to form Compound 9g: 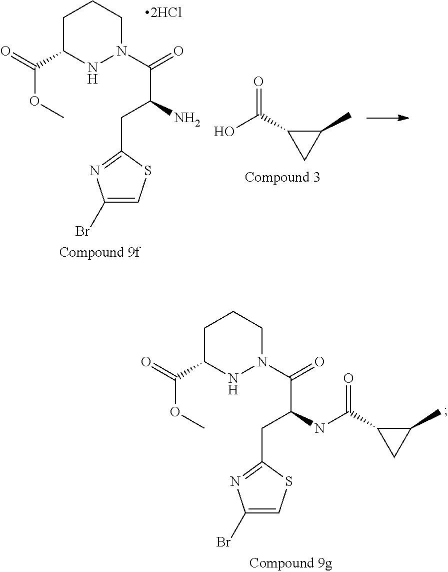
and
f) hydrolyzing Compound 9g to form Compound 9:

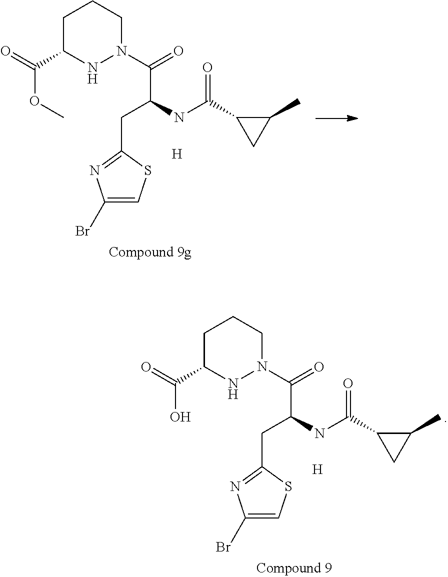

13. The method of claim 12, wherein the coupling step (a) comprises contacting Compound 2a with a zinc source to form Compound 2a-Zn:

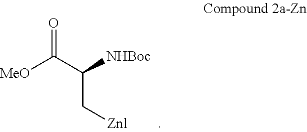

14. A method of preparing Compound A: 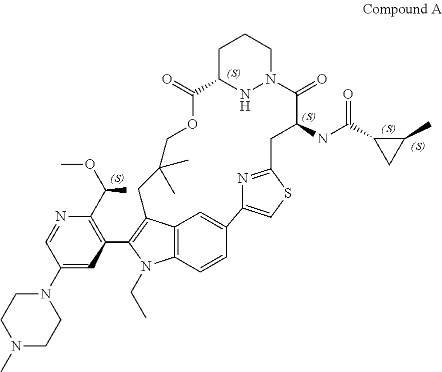
the method comprising:
a) contacting Compound 6a with pinacol to form Compound 10: 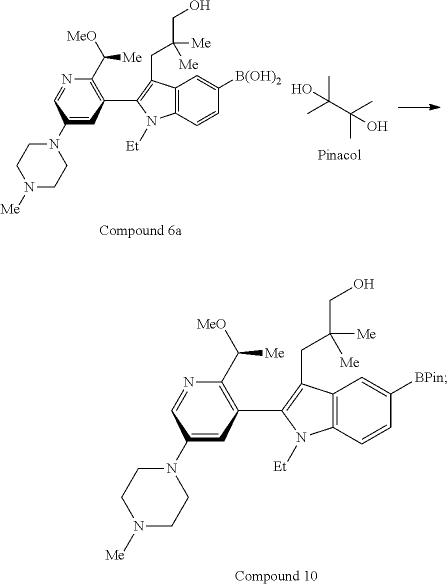
b) esterifying Compound 9 with Compound 10 to form Compound 11: 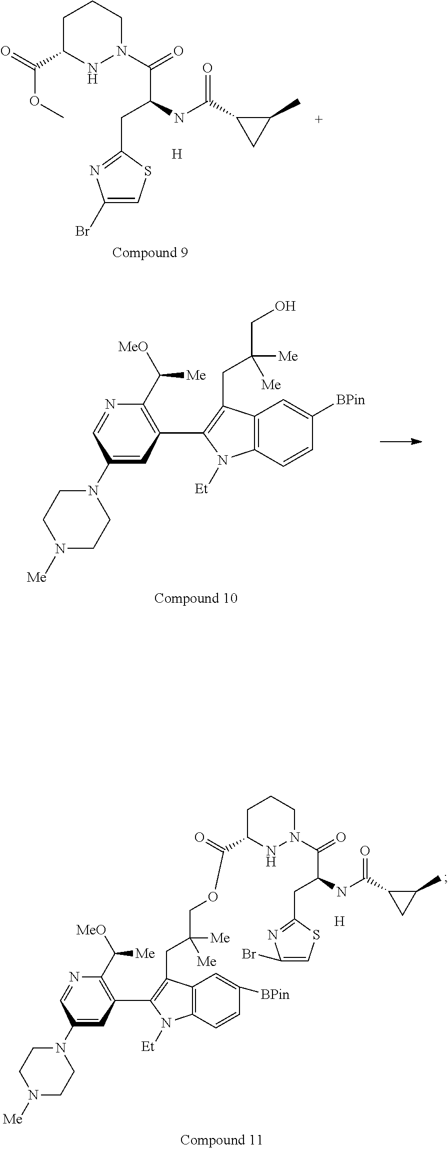
and
c) cyclizing Compound 11 to form Compound A:

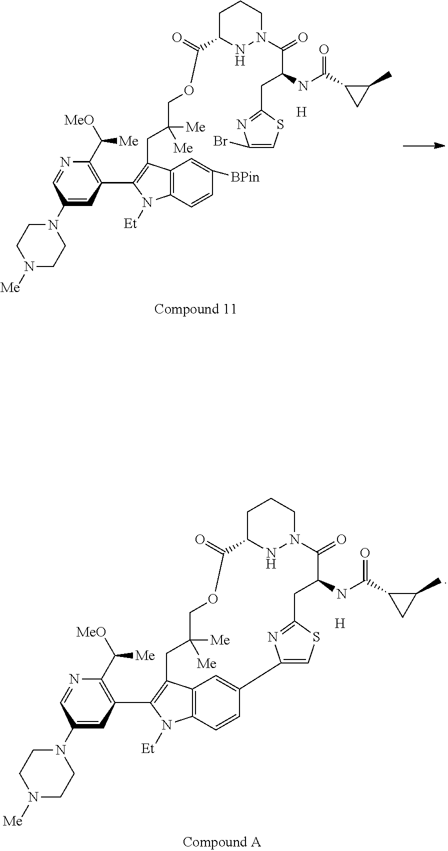

15. A method of preparing Compound A: 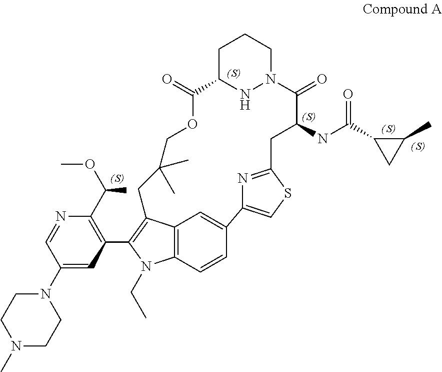
the method comprising the steps of:
a) coupling Compound 6a and Compound 9 to form Compound 12: 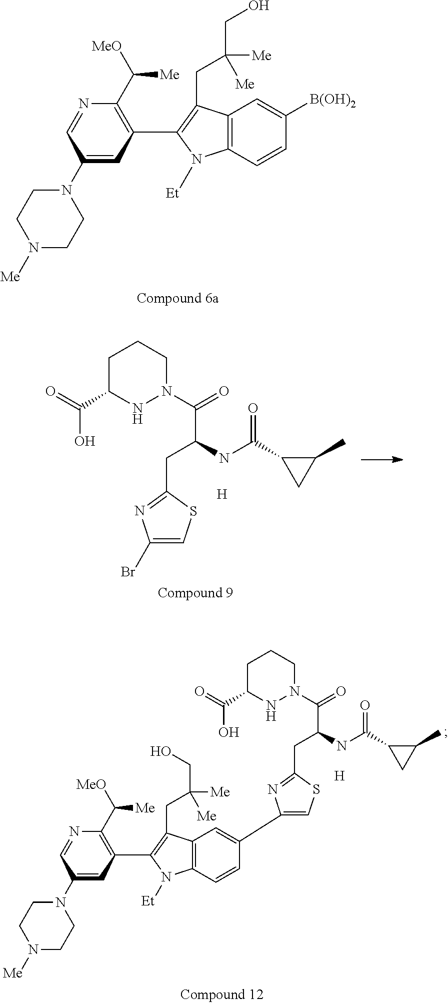
and
b) lactonizing Compound 12 to form compound A:

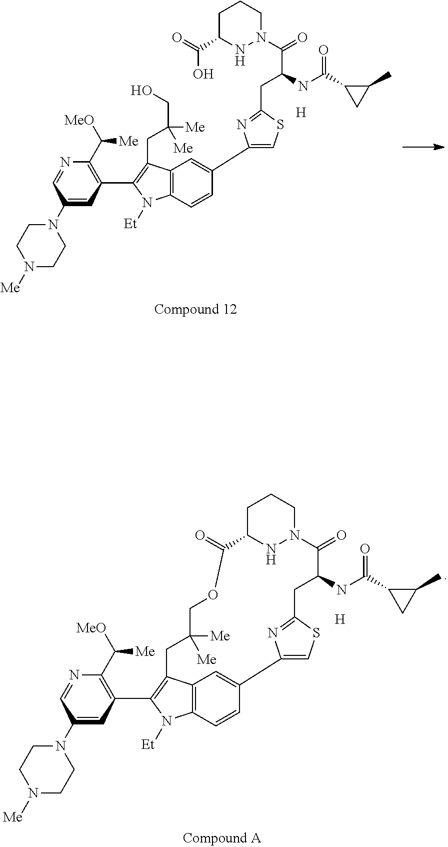

16. The method of claim 14 or 15, wherein the method further comprises the step of purifying Compound A.

17. The method of claim 16, wherein the purifying comprises forming a salt of Compound A.

18. The method of claim 17, wherein the salt of Compound A is a hydrochloride salt of Compound A or a lactate salt of Compound A.

19. The method of claim 17, wherein the purifying comprises the step of converting the salt of Compound A to a free base form of Compound A.

20. A compound having the structure of Compound 10, Compound 11, or Compound 12: 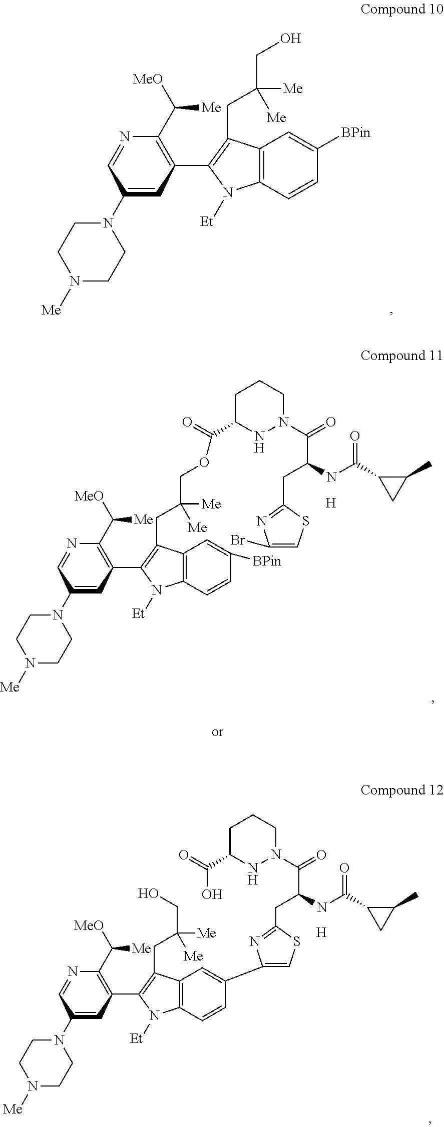
or a salt thereof.

21. The method of claim 18, wherein the purifying comprises the step of converting the hydrochloride or lactate salt of Compound A to a free base form of Compound A.
